SEQUENCE LISTING (129 sequences)

SEQ ID NO: 1 - DNA, 2605 bp, Homo sapiens
gagggcgcgtgagctggggagggcctccgagctgggtgtcgagaccctgcaggcagactggaggtgggggccaggggtaggctctgcacaccacccccccatctaccccatgactctccaccatggcccgaggtccacctggtgtcctggaagctgcccccgcccccacccagcttcctgactttggctgtgtccagagctaagaatagacgctccctgcccggcccccgccggaaaccgcagccacgggggtacatcctgagcctggcctctgttcccaccatctgccccccagcccctcacaactcctcccaccagagcctcctgtgctcccattgaacccgaagtgctggcggggggtggggggcggggggcgggccaagaccgggagaccagcaaccagccccagatgacagggaagcccagtgctcatgttccaatcccacacatgggcgtggcattgttccccctttgtgcctcagtttccccatctctaaaatttaagagtatctttaccccctgccttgcagagatgtggtgaggcttaagctaaatgcaagtgtccttctctgaactctgcccaggctgtttcccagaagagtcattgtccttgttatctcagggccgaggacagaggaagtggggacaggacagccttcctccgtctctcactgccccctcatctggagccccagccccacatcctcctcccccaacatctgccctccttgcagttagtgtagcaagggcctcatgctctctcttgcctccaaagctttatacatgcaattcctgtcactgccacgtagcccagacctggggagctcctattcatcctacaaaaccccacttcccatgccctctcctcacaagatgtcttcctagctcacctctgggcttccctaggccctgtctcttcctttggcccagccctgaccccagggggctggaacagcctgtgtccagttcattctccagcctagggcatcctgaagggcaggcctggtttcaggtctcacttggagctcataattgtcccactgtataagctgaacacagagaggggttggggagagtaagacttggagattaggtcggcttcacgggaaagcagccttggagccagggttttgccacatccataggagttggatagcctccttgccaggaaagagagaagttgtgttaaatggctggtgacttaatgggatagagctttgggagggaggttgggtgtggccgaaggccaggaggaggaaggatagtgggcggggtagccggggaaggggcttccggactgggctgacgagagggccgggccaggctgggaatacccctcactcacagcctggactagcagccctcccgggcccaggggacggggtggggtgcagtcagggcaaggtcagcttgtccggtcccactccaggtccctaactcaaggttctgtccggctcgggtgtacaaacaaggccaaggaagcccaaggtcccccctctgcaaagagacggtcccagtccctgcgtccggcaccttctcagccattcctgttgggctccatccctccttccccagcccgagctggggggtggccaggaaagagtgcccatcccccactgcagcctcccggctccagactgttcctcctctggctccaaagccacctccgagcctcctcaagagaccctcccatgcgatgccatcaccccagccctcccggctcacctgctctgggaccacctttctcagtctggctgtgtcgtctggtgcaccccccatccctctgtcctcccaccccacccctggaaggggcctcaaatatgtcctgtatgtattaagcagggcatactactactgtgtgccgcgttccagcgcacccagtgaatttggagccctggagtctgggcgacccccgggagccgatccgcctggacctgccccgccccctccaggccttgggtgctgcccagatggtgaataatgcgggcctggcggcgggagcgccgggaagaggcctggcagggcgctgggcggctggaggggctgaggctcccgaggaagcgcccctgccacccctacgcgggccattgcatccctgccaccggtggcctgggggtcccaagggcgccgccttccccagaaggctgtagggtgtcccggcgtgcggggggcacccactgactccaagtcagccaggcccgggcccgaggggcgtggccagcgcagggtgggcggggctgatgagagacagcgggagacagagactggacggagagagcgagccgcgggctgccagcggcccccacccgtcccagctcggcttagcccccgcggacccctccaggccgcgaccccagggcgtcctgtgccgccacgccctccatctgtgtgggtcctctgctgggcccgcccctggtcacagccagactgactcagtttccctgggaggtcccgctcgagcccgtccttcccctccctctgcccgcccccagccctcgccccaccctcggcgcccgcacatctgcctgctcagctccagacggcgcccggacccccgggcgcgggatccagccaggtgggagccccgcagatgaggtctctgaag

SEQ ID NO: 2 - DNA, 245 bp, Homo sapiens
gtgaggcttaagctaaatgcaagtgtccttctctgaactctgcccaggctgtttcccagaagagtcattgtccttgttatctcagggccgaggacagaggaagtggggacaggacagccttcctccgtctctcactgccccctcatctggagccccagccccacatcctcctcccccaacatctgccctccttgcagttagtgtagcaagggcctcatgctctctcttgcctccaaagctttata

SEQ ID NO: 3 - DNA, 213 bp, Homo sapiens
catgcaattcctgtcactgccacgtagcccagacctggggagctcctattcatcctacaaaaccccacttcccatgccctctcctcacaagatgtcttcctagctcacctctgggcttccctaggccctgtctcttcctttggcccagccctgaccccagggggctggaacagcctgtgtccagttcattctccagcctagggcatcctgaag

SEQ ID NO: 4 - DNA, 537 bp, Homo sapiens
gagggcgcgtgagctggggagggcctccgagctgggtgtcgagaccctgcaggcagactggaggtgggggccaggggtaggctctgcacaccacccccccatctaccccatgactctccaccatggcccgaggtccacctggtgtcctggaagctgcccccgcccccacccagcttcctgactttggctgtgtccagagctaagaatagacgctccctgcccggcccccgccggaaaccgcagccacgggggtacatcctgagcctggcctctgttcccaccatctgccccccagcccctcacaactcctcccaccagagcctcctgtgctcccattgaacccgaagtgctggcggggggtggggggcggggggcgggccaagaccgggagaccagcaaccagccccagatgacagggaagcccagtgctcatgttccaatcccacacatgggcgtggcattgttccccctttgtgcctcagtttccccatctctaaaatttaagagtatctttaccccctgccttgcagagatgtg

SEQ ID NO: 5 - DNA, 200 bp, Homo sapiens
gctccaaagccacctccgagcctcctcaagagaccctcccatgcgatgccatcaccccagccctcccggctcacctgctctgggaccacctttctcagtctggctgtgtcgtctggtgcaccccccatccctctgtcctcccaccccacccctggaaggggcctcaaatatgtcctgtatgtattaagcagggcatacta

SEQ ID NO: 6 - DNA, 136 bp, Homo sapiens
tgagagacagcgggagacagagactggacggagagagcgagccgcgggctgccagcggcccccacccgtcccagctcggcttagcccccgcggacccctccaggccgcgaccccagggcgtcctgtgccgccacgc

SEQ ID NO: 7 - DNA, 425 bp, Homo sapiens
actccaagtcagccaggcccgggcccgaggggcgtggccagcgcagggtgggcggggctgatgagagacagcgggagacagagactggacggagagagcgagccgcgggctgccagcggcccccacccgtcccagctcggcttagcccccgcggacccctccaggccgcgaccccagggcgtcctgtgccgccacgccctccatctgtgtgggtcctctgctgggcccgcccctggtcacagccagactgactcagtttccctgggaggtcccgctcgagcccgtccttcccctccctctgcccgcccccagccctcgccccaccctcggcgcccgcacatctgcctgctcagctccagacggcgcccggacccccgggcgcgggatccagccaggtgggagccccgcagatgaggtctctgaag

SEQ ID NO: 8 - DNA, 15 bp, Homo sapiens
tgcccccgcccccac

SEQ ID NO: 9 - DNA, 15 bp, Homo sapiens
tgcccctgaccccac

SEQ ID NO: 10 - DNA, 17 bp, Homo sapiens
gcccggcccccgccgga

SEQ ID NO: 11 - DNA, 17 bp, Homo sapiens
gcccggttcccgccgga

SEQ ID NO: 12 - DNA, 29 bp, Homo sapiens
cggggggtggggggcggggggcgggccaa

SEQ ID NO: 13 - DNA, 29 bp, Homo sapiens
cgggggtcgtggggtggatggcgggccaa

SEQ ID NO: 14 - DNA, 17 bp, Homo sapiens
tcactgccccctcatct

SEQ ID NO: 15 - DNA, 17 bp, Homo sapiens
tcactgtcctctcatct

SEQ ID NO: 16 - DNA, 13 bp, Homo sapiens
cttgttatctcag

SEQ ID NO: 17 - DNA, 13 bp, Homo sapiens
cttggtagctcag

SEQ ID NO: 18 - DNA, 17 bp, Homo sapiens
gacagaggaagtgggga

SEQ ID NO: 19 - DNA, 17 bp, Homo sapiens
gacaggtgaagtgggga

SEQ ID NO: 20 - DNA, 17 bp, Homo sapiens
gccccacatcctcctcc

SEQ ID NO: 21 - DNA, 17 bp, Homo sapiens
gccccacatcaccctcc

SEQ ID NO: 22 - DNA, 17 bp, Homo sapiens
tctgtcctcccacccca

SEQ ID NO: 23 - DNA, 17 bp, Homo sapiens
tctgtcctatcacccca

SEQ ID NO: 24 - DNA, 15 bp, Homo sapiens
cgaggggcgtggcca

SEQ ID NO: 25 - DNA, 15 bp, Homo sapiens
cgaggaacatggcca

SEQ ID NO: 26 - DNA, 19 bp, Homo sapiens
cgcagggtgggcggggctg

SEQ ID NO: 27 - DNA, 19 bp, Homo sapiens
cgcagggtgtatggggctg

SEQ ID NO: 28 - DNA, 17 bp, Homo sapiens
cagcggcccccacccgt

SEQ ID NO: 29 - DNA, 17 bp, Homo sapiens
cagcggcctacacccgt

SEQ ID NO: 30 - DNA, 15 bp, Homo sapiens
tgggcccgcccctgg

SEQ ID NO: 31 - DNA, 15 bp, Homo sapiens
tgggcccaatcctgg

SEQ ID NO: 32 - DNA, 21 bp, Homo sapiens
ccagactgactcagtttccct

SEQ ID NO: 33 - DNA, 21 bp, Homo sapiens
ccagacacactcagtttccct

SEQ ID NO: 34 - DNA, 19 bp, Homo sapiens
tctgcccgcccccagccct

SEQ ID NO: 35 - DNA, 19 bp, Homo sapiens
tctgccctaacccagccct

SEQ ID NO: 36 - DNA, 18 bp, Homo sapiens
ccctcgccccaccctcgg

SEQ ID NO: 37 - DNA, 18 bp, Homo sapiens
ccctcgcaataccctcgg

SEQ ID NO: 38 - DNA, 34 bp, Artificial Sequence
  Synthetic Primer Sequence
gagaggtacctgagagacagcgggagacagagac

SEQ ID NO: 39 - DNA, 33 bp, Artificial sequence
  Synthetic Primer Sequence
agagacgcgtcttcagagacctcatctgcgggg

SEQ ID NO: 40 - DNA, 31 bp, Artificial Sequence
  Synthetic Primer Sequence
gagaggtacccctccatctgtgtgggtcctc

SEQ ID NO: 41 - DNA, 28 bp, Artificial Sequence
  Synthetic Primer Sequence
gagaggtacctccaggccttgggtgctg

SEQ ID NO: 42 - DNA, 32 bp, Artificial Sequence
  Synthetic Primer Sequence
gagaaagcttttatgaaacattcagtggggag

SEQ ID NO: 43 - DNA, 41 bp, Artificial Sequence
  Synthetic Primer Sequence
gatgttccagattacgctagcctctgggtctgctgcctgtg

SEQ ID NO: 44 - DNA, 30 bp, Artificial Sequence
  Synthetic Primer Sequence
tggaagctgcccctgaccccacccagcttc

SEQ ID NO: 45 - DNA, 27 bp, Artificial Sequence
  Synthetic Primer Sequence
ctccctgcccggttcccgccggaaacc

SEQ ID NO: 46 - DNA, 30 bp, Artificial Sequence
  Synthetic Primer Sequence
gtgctggcgggggtcgtggggcggggggcg

SEQ ID NO: 47 - DNA, 30 bp, Artificial Sequence
  Synthetic Primer Sequence
ggggtcgtggggtggatggcgggccaagac

SEQ ID NO: 48 - DNA, 32 bp, Artificial Sequence
  Synthetic Primer Sequence
tccgtctctcactgtcctctcatctggagccc

SEQ ID NO: 49 - DNA, 30 bp, Artificial Sequence
  Synthetic Primer Sequence
catccctctgtcctatcaccccacccctgg

SEQ ID NO: 50 - DNA, 28 bp, Artificial Sequence
  Synthetic Primer Sequence
gctgccagcggcctacacccgtcccagc

SEQ ID NO: 51 - DNA, 27 bp, Artificial Sequence
  Synthetic Primer Sequence
ccatgcgatgccaccaccccagccctc

SEQ ID NO: 52 - DNA, 91 bp, Artificial Sequence
  Synthetic Primer Sequence
gaagctgcccccgcccccacccagcttcctgactttggctgtgtccagagctaagaatagacgctccctgcccggcccccgccggaaaccg

SEQ ID NO: 53 - DNA, 42 bp, Artificial Sequence
  Synthetic Primer Sequence
gtgctggcggggggtggggggcggggggcgggccaagaccgg

SEQ ID NO: 54 - DNA, 38 bp, Artificial Sequence
  Synthetic Primer Sequence
tcctccgtctctcactgccccctcatctggagccccag

SEQ ID NO: 55 - DNA, 40 bp, Artificial Sequence
  Synthetic Primer Sequence
caccccccatccctctgtcctcccaccccacccctggaag

SEQ ID NO: 56 - DNA, 40 bp, Artificial Sequence
  Synthetic Primer Sequence
gccgcgggctgccagcggcccccacccgtcccagctcggc

SEQ ID NO: 57 - DNA, 27 bp, Artificial Sequence
  Synthetic Primer Sequence
ctggaagctgcccccgcccccacccag

SEQ ID NO: 58 - DNA, 29 bp, Artificial Sequence
  Synthetic Primer Sequence
ctccctgcccggcccccgccggaaaccgc

SEQ ID NO: 59 - DNA, 42 bp, Artificial Sequence; identical to SEQ ID NO: 53
  Synthetic Primer Sequence

SEQ ID NO: 60 - DNA, 38 bp, Artificial Sequence; identical to SEQ ID NO: 54
  Synthetic Primer Sequence

SEQ ID NO: 61 - DNA, 40 bp, Artificial Sequence; identical to SEQ ID NO: 55
  Synthetic Primer Sequence

SEQ ID NO: 62 - DNA, 40 bp, Artificial Sequence; identical to SEQ ID NO: 56
  Synthetic Primer Sequence

SEQ ID NO: 63 - DNA, 21 bp, Artificial Sequence
  Synthetic Primer Sequence
cgagtgcgtgggagtagaatt

SEQ ID NO: 64 - DNA, 22 bp, Artificial Sequence
  Synthetic Primer Sequence
attcgatcggggcggggcgagc

SEQ ID NO: 65 - DNA, 27 bp, Artificial Sequence
  Synthetic Primer Sequence
ggatccagcgggggcgagcgggggcga

SEQ ID NO: 66 - DNA, 28 bp, Artificial Sequence
  Synthetic Primer Sequence
gggccgaggacaggtgaagtggggacag

SEQ ID NO: 67 - DNA, 21 bp, Artificial Sequence
  Synthetic Primer Sequence
gccagactgactcagtttccc

SEQ ID NO: 68 - DNA, 24 bp, Artificial Sequence
  Synthetic Primer Sequence
cgagaccctgcaggcagactggag

SEQ ID NO: 69 - DNA, 25 bp, Artificial Sequence
  Synthetic Primer Sequence
gagatggggaaactgaggcacaaag

SEQ ID NO: 70 - DNA, 23 bp, Artificial Sequence
  Synthetic Primer Sequence
gccttgcagagatgtggtgaggc

SEQ ID NO: 71 - DNA, 24 bp, Artificial Sequence
  Synthetic Primer Sequence
gaggtgagctaggaagacatcttg

SEQ ID NO: 72 - DNA, 28 bp, Artificial Sequence
  Synthetic Primer Sequence
gagaggtaccgctccaaagccacctccg

SEQ ID NO: 73 - DNA, 29 bp, Artificial Sequence
  Synthetic Primer Sequence
agagacgcgtcgcttcctcgggagcctca

SEQ ID NO: 74 - DNA, 25 bp, Artificial Sequence
  Synthetic Primer Sequence
cttccccagaaggctgtagggtgtc

SEQ ID NO: 75 - DNA, 20 bp, Artificial Sequence
  Synthetic Primer Sequence
gagatgatggctcagctcct

SEQ ID NO: 76 - DNA, 18 bp, Artificial Sequence
  Synthetic Primer Sequence
ccagcccctgaatcctca

SEQ ID NO: 77 - DNA, 19 bp, Artificial Sequence
  Synthetic Primer Sequence
ccacagtccatgccatcac

SEQ ID NO: 78 - DNA, 18 bp, Artificial Sequence
  Synthetic Primer Sequence
tgaaggtcggagtcaacg

SEQ ID NO: 79 - DNA, 18 bp, Artificial Sequence
  Synthetic Primer Sequence
catgtgggccatgaggtc

SEQ ID NO: 80 - DNA, 21 bp, Artificial Sequence
  Synthetic Primer Sequence
ctgtcccgcaccacggagaag

SEQ ID NO: 81 - DNA, 22 bp, Artificial Sequence
  Synthetic Primer Sequence
cacccagaagactgtggatggc

SEQ ID NO: 82 - DNA, 30 bp, Artificial Sequence
  Synthetic Primer Sequence
gagaggtaccgtgaggcttaagctaaatgc

SEQ ID NO: 83 - DNA, 34 bp, Artificial Sequence
  Synthetic Primer Sequence
gagaggtacctggggacaggacagccttcctccg

SEQ ID NO: 84 - DNA, 31 bp, Artificial Sequence
  Synthetic Primer Sequence
gagaggtacccatgcaattcctgtcactgcc

SEQ ID NO: 85 - DNA, 32 bp, Artificial Sequence
  Synthetic Primer Sequence
gagaggtaccggcaggcctggtttcaggtctc

SEQ ID NO: 86 - DNA, 28 bp, Artificial Sequence
  Synthetic Primer Sequence
gagaggtaccctactgtgtgccgcgttc

SEQ ID NO: 87 - DNA, 31 bp, Artificial Sequence
  Synthetic Primer Sequence
gagaggtaccgactccaagtcagccaggccc

SEQ ID NO: 88 - DNA, 31 bp, Artificial Sequence
  Synthetic Primer Sequence
gagaggtacctcagtttccctgggaggtccc

SEQ ID NO: 89 - DNA, 29 bp, Artificial Sequence
  Synthetic Primer Sequence
gagaggtacccagccctcgccccaccctc

SEQ ID NO: 90 - DNA, 34 bp, Artificial Sequence
  Synthetic Primer Sequence
agagacgcgttataaagctttggaggcaagagag

SEQ ID NO: 91 - DNA, 32 bp, Artificial Sequence
  Synthetic Primer Sequence
gtcattgtccttggtagctcagggccgaggac

SEQ ID NO: 92 - DNA, 27 bp, Artificial Sequence
  Synthetic Primer Sequence
cccagccccacatcaccctcccccaac

SEQ ID NO: 93 - DNA, 33 bp, Artificial Sequence
  Synthetic Primer Sequence
ctggtcacagccagacacactcagtttccctgg

SEQ ID NO: 94 - DNA, 30 bp, Artificial Sequence
  Synthetic Primer Sequence
cccgggcccgaggaacatggccagcgcagg

SEQ ID NO: 95 - DNA, 33 bp, Artificial Sequence
  Synthetic Primer Sequence
gccagcgcagggtgtatggggctgatgagagac

SEQ ID NO: 96 - DNA, 32 bp, Artificial Sequence
  Synthetic Primer Sequence
cctctgctgggcccaatcctggtcacagccag

SEQ ID NO: 97 - DNA, 30 bp, Artificial Sequence
  Synthetic Primer Sequence
cctccctctgccctaacccagccctcgccc

SEQ ID NO: 98 - DNA, 30 bp, Artificial Sequence
  Synthetic Primer Sequence
cccagccctcgcaataccctcggcgcccgc

SEQ ID NO: 99 - DNA, 39 bp, Artificial Sequence
  Synthetic Primer Sequence
ctggtcacagccagactgactcagtttccctgggaggtc

SEQ ID NO: 100 - DNA, 24 bp, Artificial Sequence
  Synthetic Primer Sequence
gggcccgaggggcgtggccagcgc

SEQ ID NO: 101 - DNA, 29 bp, Artificial Sequence
  Synthetic Primer Sequence
gccagcgcagggtgggcggggctgatgag

SEQ ID NO: 102 - DNA, 43 bp, Artificial Sequence
  Synthetic Primer Sequence
ctccctctgcccgcccccagccctcgccccaccctcggcgccc

SEQ ID NO: 103 - DNA, 50 bp, Artificial Sequence
  Synthetic Primer Sequence
cattgtccttgttatctcagggccgaggacagaggaagtggggacaggac

SEQ ID NO: 104 - DNA, 39 bp, Artificial Sequence; identical to SEQ ID NO: 99
  Synthetic Primer Sequence

SEQ ID NO: 105 - DNA, 27 bp, Artificial Sequence
  Synthetic Primer Sequence
tggggaacctgtgctgagtcactggag

SEQ ID NO: 106 - DNA, 21 bp, Artificial Sequence
  Synthetic Primer Sequence
cgcttgatgactcagccggaa

SEQ ID NO: 107 - DNA, 24 bp, Artificial Sequence
  Synthetic Primer Sequence
gggcccgaggggcgtggccagcgc

SEQ ID NO: 108 - DNA, 29 bp, Artificial Sequence
  Synthetic Primer Sequence
gccagcgcagggtgggcggggctgatgag

SEQ ID NO: 109 - DNA, 26 bp, Artificial Sequence
  Synthetic Primer Sequence
ctccctctgcccgcccccagccctcg

SEQ ID NO: 110 - DNA, 30 bp, Artificial Sequence
  Synthetic Primer Sequence
ccccagccctcgccccaccctcggcgcccg

SEQ ID NO: 111 - DNA, 27 bp, Artificial Sequence
  Synthetic Primer Sequence
cattgtccttgttatctcagggccgag

SEQ ID NO: 112 - DNA, 30 bp, Artificial Sequence
  Synthetic Primer Sequence
ggccgaggacagaggaagtggggacaggac

SEQ ID NO: 113 - DNA, 18 bp, Artificial Sequence
  Synthetic Primer Sequence
tccctgataagacccagg

SEQ ID NO: 114 - DNA, 21 bp, Artificial Sequence
  Synthetic Primer Sequence
gatctcgagcaggaagttcga

SEQ ID NO: 115 - DNA, 27 bp, Artificial Sequence
  Synthetic Primer Sequence
tgcgcccggccttccatgctctttgac

SEQ ID NO: 116 - DNA, 17 bp, Homo sapiens
gtgcgtgggagtagaat

SEQ ID NO: 117 - DNA, 10 bp, Homo sapiens
ggggcggggc

SEQ ID NO: 118 - DNA, 23 bp, Homo sapiens
ctgggtgggggcgggggcagctt

SEQ ID NO: 119 - DNA, 19 bp, Homo sapiens
tccggcgggggccgggcag

SEQ ID NO: 120 - DNA, 31 bp, Homo sapiens
ggcggggggtggggggcggggggcgggccaa

SEQ ID NO: 121 - DNA, 17 bp, Homo sapiens
agatgagggggcagtga

SEQ ID NO: 122 - DNA, 24 bp, Homo sapiens
ccaggggtggggtgggaggacaga

SEQ ID NO: 123 - DNA, 17 bp, Homo sapiens
acgggtgggggccgctg

SEQ ID NO: 124 - DNA, 954 bp, Homo sapiens
gctccaaagccacctccgagcctcctcaagagaccctcccatgcgatgccaccaccccagccctcccggctcacctgctctgggaccacctttctcagtctggctgtgtcgtctggtgcaccccccatccctctgtcctatcaccccacccctggaaggggcctcaaatatgtcctgtatgtattaagcagggcatactactactgtgtgccgcgttccagcgcacccagtgaatttggagccctggagtctgggcgacccccgggagccgatccgcctggacctgccccgccccctccaggccttgggtgctgcccagatggtgaataatgcgggcctggcggcgggagcgccgggaagaggcctggcagggcgctgggcggctggaggggctgaggctcccgaggaagcgcccctgccacccctacgcgggccattgcatccctgccaccggtggcctgggggtcccaagggcgccgccttccccagaaggctgtagggtgtcccggcgtgcggggggcacccactgactccaagtcagccaggcccgggcccgaggggcgtggccagcgcagggtgggcggggctgatgagagacagcgggagacagagactggacggagagagcgagccgcgggctgccagcggcctacacccgtcccagctcggcttagcccccgcggacccctccaggccgcgaccccagggcgtcctgtgccgccacgccctccatctgtgtgggtcctctgctgggcccgcccctggtcacagccagactgactcagtttccctgggaggtcccgctcgagcccgtccttcccctccctctgcccgcccccagccctcgccccaccctcggcgcccgcacatctgcctgctcagctccagacggcgcccggacccccgggcgcgggatccagccaggtgggagccccgcagatgaggtctctgaag

SEQ ID NO: 125 - DNA, 954 bp, Homo sapiens
  CDS (123)..(953)
attaatacgactcactatagggagacccaagctggctagcgtttaaacgggccctctagactcgagcggccgccactgtgctggatatctgcagaattccaccacactggactagtggatccatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgta

SEQ ID NO: 126 - protein, 277 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKC

SEQ ID NO: 127 - DNA, 896 bp, Homo sapiens
cttttatgaaacattcagtggggagttctccttccagctctgccccactcctgcctttctacacttgtgtagagatatagccaggatgatgacaaggatgatgaccacaaataccacagctccaatgatgtagaatatttctctgaattcccctttctcctggcccatacactctaccgggctgtctgtactcttccggttaactgttcgggagggaatcactgcttgaacactgaaacagtagttttctcctttatccacatcaatcaaaaactcattagtgtttgttttggctgttttctttcctgaacttgaagatttccaataataaagtgtataaattaagtccttgccaaaaacatcccggaggcttaggaaagtgttgttccttctgactaaagtccgttcatcttctacggtcacattcacttttgttcccacctgttcaaaactctgaattgttggctgtccgaggtttgtctccaggtaaggtgtgaactctggggagttctcatacagaggctccccagcagaaccggtgctctccacattccctgccgggtaggagaagacccgtgccaagtacgtctgcttcacatccttcacaatctcgtcggtgaggtcacactctgtgtctgttgtgtaaaagcatttgcttttccaatctcctgacttagtgcttatttgaacagtgtagacttgattgacgggtttgggttcccactccaaaattgtcttgaaattagttgatttccaagttaaattatatgctgccacagtatttgtagtgcctgaagcgccggccacctgggcgaagacccagccgagcaggagcgtccgagcgacggcggtctcggggcgcgggacccggggccaggcaggggtctccatggatc

SEQ ID NO: 128 - DNA, 896 bp, Homo sapiens
  CDS (6)..(896)
gatccatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaag

SEQ ID NO: 129 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

(19) United States
(12) Patent Application Publication
(10) Pub. No.: US 2011/0269825 A1
(43) Pub. Date: Nov. 3, 2011

(54) Modified Thromboxane A2 Receptor Promoter Sequence

(76) Inventors: Therese Kinsella, County Dublin (IE); AnneMarie Gannon, County Galway (IE); Elizabeth Turner, Dublin (IE); Garrett Keating, Dublin (IE)

(21) Appl. No.: 13/058,716

(22) PCT Filed: Aug. 11, 2009
(86) PCT No.: PCT/EP2009/005822

§ 371 Date: Mar. 25, 2011

(30) Foreign Application Priority Data
Aug. 11, 2008 (GB) 0814615.1

(51) Int. Cl. A61K 31/7088; A61P 43/00; C07H 21/04; C12Q 1/68; C12N 5/02

(52) U.S. Cl. 514/44 R; 435/6.1; 435/6.14; 435/375; 536/24.1

(57) ABSTRACT

The invention provides nucleic acid sequences useful in regulating the transcription of a gene. In particular, the invention relates to a promoter sequence, and variants thereof, that can be used to differentially regulate the transcription of a gene. The present invention, accordingly, provides methods for regulating transcription of a gene, the method comprising providing a gene transcription-regulating polynucleotide in operable association with the gene, optionally within a host cell, wherein the gene transcription-regulating polynucleotide comprises the nucleic acid sequence of SEQ ID NO:1 of the nucleic acid sequence of thromboxane A2 receptor promoter or a fragment thereof, the gene transcription-regulating polynucleotide or the fragment thereof further comprising at least one nucleic acid modification and/or substitution. The nucleic acid sequences and probes of the present invention also find utility as predictive markers for alterations in gene transcription in disease settings; or can be used to achieve over-expression of recombinant proteins in mammalian cells. Accordingly, the present invention also provides recombinant expression vectors and host cells for use in regulating the transcription of a gene.

This invention relates to nucleic acid sequences useful in regulating the transcription of a gene. In particular, the invention relates to a promoter sequence, and variants thereof, that can be used to differentially regulate the transcription of a gene, and/or which are predictive markers for alterations in gene transcription in disease settings, and/or which can be used to achieve over-expression of recombinant proteins in mammalian cells.

### BACKGROUND

The prostanoid thromboxane (TX) A₂ plays a central role in haemostasis and vascular tone, acting as a potent mediator of platelet activation and aggregation, and potentiation of mitogenic or hypertrophic growth of vascular smooth muscle cells. TXA₂ is mainly produced in platelets, vascular smooth muscle cells, activated macrophages and monocytes, and imbalances in the level of TXA₂, or its receptor, are associated with a variety of vascular disorders such as thrombosis, various hypertensions, atherosclerosis, and ischaemic heart disease, as well as with inflammatory renal diseases. In humans, TXA₂ signals through two TXA₂ receptor (TP) isoforms, termed TPα and TPβ, that are encoded by a single gene on chromosome 19p13.3, but are transcriptionally regulated by distinct promoters, termed Prm1 and Prm3, respectively. TPα and TPβ, are identical for their N-terminal 328 amino acid residues but differ exclusively in their C-tail domains. As members of the G protein coupled receptor (GPCR) superfamily. TPα and TPβ show identical coupling to Gα_{q}-mediated phospholipase Cβ activation, but differentially couple to other secondary effectors including adenylyl cyclase and tissue transglutaminase. TPα and TPβ regulate both common and distinct signaling pathways but are subject to entirely different modes of regulation, such as through both agonist-dependent (homologous) desensitization and through intramolecular cross-talk between other signaling systems. Hence, while the functional relevance for the existence of two TP receptors in primates is currently unknown, there is abundant evidence that they have distinct physiologic roles. As well as being subject to differential regulation, the relative expression of TPα and TPβ mRNA varies greatly in a range of cell and tissues types of vascular origin. The findings that platelets almost exclusively express TPα, and that anti-aggregatory autocoids, including prostacyclin and nitric oxide, act as mediators of TPα desensitization, suggest that TPα may be the isoform that plays a more central role in haemostasis. Hence, whilst the significance of two receptors for TXA₂ in humans but not in other species is currently unknown, there is abundant and increasing evidence that they have distinct (patho)physiologic roles displaying differences in their signalling, modes of regulation and patterns of expression. Despite the recognized importance of TPα in haemostasis, contributing to platelet activation status and vascular tone, until recently the factors regulating its expression through Prm1 remained largely uncharacterised. Prm1 is known to lack consensus TATA or CAAT elements, and transcription initiation is thought to occur at multiple sites within exon (E)1 of the TP gene. Moreover, in the platelet progenitor megakaryocytic HEL 92.1.7 cell line, Prm1 is known to be located between nucleic acid positions −8500 and −5895, and in this invention, it has been discovered that it can be regulated by binding of transcription factors such as Sp1, Egr1, NF-E2, GATA, Ets and WT1.

### SUMMARY OF THE INVENTION

According to a first aspect of the present invention, there is described a gene transcription-regulating polynucleotide selected from the nucleic acid sequence of at least one, optionally any one, of SEQ ID NOs:2-6, optionally, comprising at least one nucleic acid modification and/or substitution; the nucleic acid sequence of a fragment of SEQ ID NO:1 of the nucleic acid sequence of thromboxane A2 receptor promoter, optionally, comprising at least one nucleic acid modification and/or substitution; or the nucleic acid sequence of SEQ ID NO:1 of the nucleic acid sequence of thromboxane A2 receptor promoter comprising at least one nucleic acid modification and/or substitution.

It is understood that a fragment may comprise any polynucleotide comprising a nucleic acid sequence, which retains promoter activity. Preferably, the fragment is capable of promoting expression of a gene in operable association with the fragment. Further preferably, the fragment is a functional fragment, which retains at least part of the promoter activity of the gene transcription-regulating polynucleotide.

Also encompassed within the scope of the first aspect of the present invention is a method for regulating transcription of a gene, the method comprising providing a gene transcription-regulating polynucleotide in operable association with the gene, optionally within a host cell, wherein the gene transcription-regulating polynucleotide is selected from the nucleic acid sequence of at least one, optionally any one, of SEQ ID NOs:2-6, optionally, comprising at least one nucleic acid modification and/or substitution; the nucleic acid sequence of SEQ ID NO:1 of the nucleic acid sequence of thromboxane A2 receptor promoter, optionally, comprising at least one nucleic acid modification and/or substitution; or the nucleic acid sequence of a fragment of SEQ ID NO:1 of the nucleic acid sequence of thromboxane A2 receptor promoter comprising at least one nucleic acid modification and/or substitution.

It is understood that the gene transcription-regulating polynucleotide in operable association with the gene can be in the presence of those biological components necessary to promote expression of the gene, and optionally to initiate and maintain the transcription process. For example, those biological components may comprise transcription factors, polymerase enzymes, or other biological entities necessary for transcription of the gene. Those biological components may be provided, for example, within a host cell, or in a cell-free system or subcellular fraction.

According to a second aspect of the present invention, there is provided a method of producing a promoter, the method comprising the step of:
introducing at least one nucleic acid modification and/or substitution within SEQ ID NO:1 of the nucleic acid sequence of thromboxane A2 receptor promoter;
removing at least one nucleic acid from SEQ ID NO:1 of the nucleic acid sequence of thromboxane A2 receptor promoter;
introducing at least one further nucleic acid into SEQ ID NO:1 of the nucleic acid sequence of thromboxane A2 receptor promoter; or
a combination thereof.

According to a third aspect of the present invention there is described the use of a polynucleotide for regulating gene transcription, the polynucleotide comprising the nucleic acid sequence of SEQ ID NO:1 of the nucleic acid sequence of thromboxane A2 receptor promoter or a fragment thereof, the gene transcription-regulating polynucleotide or the fragment thereof further comprising at least one nucleic acid modification and/or substitution.

The nucleic acid sequences of each of SEQ ID NOs: 2-6 are each wholly contained within SEQ ID NO:1. Optionally or additionally, the gene transcription-regulating polynucleotide fragment is selected from at least one of SEQ ID NOs: 2-6. Further optionally or additionally, the fragment comprises at least one nucleic acid modification and/or substitution. The at least one nucleic acid modification and/or substitution may, optionally, be located within any one of SEQ ID NOs:2-6

Alternatively, the at least one nucleic acid modification and/or substitution is located within SEQ ID NO:1 or a fragment thereof but outside any of SEQ ID NOs:2-6. By outside any one of SEQ ID NOs:2-6 is meant the at least one nucleic acid modification and/or substitution is located within SEQ ID NO:1 or a fragment thereof; but is not located within any one of SEQ ID NOs:2-6. Preferably, the at least one nucleic acid modification and/or substitution is located within SEQ ID NO:1 or a fragment thereof; but is, optionally, not located within at least one, optionally any one, of SEQ ID NOs:2-6. Alternatively, the at least one nucleic acid modification and/or substitution is located within SEQ ID NO:1 or a fragment thereof; and is, optionally, located within at least one, optionally any one, of SEQ ID NOs:2-6.

Optionally, the at least one nucleic acid modification and/or substitution, or the at least one further nucleic acid modification and/or substitution, is selected from the addition of at least one nucleic acid, the deletion (removal) of at least one nucleic acid, or the substitution of at least one nucleic acid.

Optionally, the at least one nucleic acid modification and/or substitution or the further nucleic acid is introduced at least one location within SEQ ID NO:1 of the nucleic acid sequence of thromboxane A2 receptor promoter, each said location being independently selected from one or more of the group comprising, but not limited to, locations whose 5′ most nucleotides are at or adjacent nucleic acid positions −6007, −6022, −6080, −6098, −6206, −6278, −6294, −6717, −7805, −7870, −7890, −7831, −8146, −8281 and −8345. All positions with respect to ATG start site (at +1) of the TP gene, where each actual nucleotide number indicated represents the 5′ nucleotide of the respective consensus transcription binding site of the wild type sequences (Table 1).

Further optionally, the at least one nucleic acid modification and/or substitution or the further nucleic acid is introduced at least one location within SEQ ID NO:1 of the nucleic acid sequence of thromboxane A2 receptor promoter, each said location being independently selected from one or more of the group comprising, but not limited to, locations whose 5′ most nucleotides are at or adjacent nucleic acid positions −8500, −7962, −7717, −6848, and −6320.

Optionally or additionally, the at least one nucleic acid modification and/or substitution is introduced; the at least one nucleic acid is removed; or the at least one further nucleic acid is introduced, into at least one element, each said element being independently selected from one or more of the group comprising, but not limited to, SEQ ID NOs:2-6.

The nucleic acid modification is selected from the group comprising, but not limited to, a multiplication (or insertion) of at least one nucleic acid or element, a deletion of at least one nucleic acid or element, inversion of the element, and a nucleic acid substitution or modification within the element. The nucleic acid modification is selected from the group comprising, but not limited to, a multiplication (or insertion) of the element, a deletion of the element, inversion of the element, and a nucleic acid substitution or modification within the element.

Optionally, the at least one nucleic acid modification and/or substitution is introduced at least one location within SEQ ID NO:1. For the purposes of the present specification, the term “location” is intended to encompass an element of the nucleic acid sequence, which element comprises at least one nucleic acid. In the case of an element comprising a single nucleic acid, the element is identified by the nucleic acid position defined in SEQ ID NO:1. Optionally, the element may comprise a string of nucleic acids. In the case of an element comprising a string of nucleic acids, the element is identified by a 5′ nucleic acid position and a 3′ nucleic acid position, the nucleic acid positions being those positions defined in SEQ ID NO:1.

Preferably, either the at least one nucleic acid modification or substitution or the at least one further nucleic acid is introduced at a location or within an element, whereby the at least one nucleic acrd modification or substitution renders the promoter non-functional.

Optionally, the element comprises a transcription factor binding site. Optionally, the element comprises a binding site for a transcription factor selected from the group comprising, but not limited to, GC, GATA, Ets, Sp1, Egr1, NF-E2, WT-1, and AP1. Preferably, the element comprises a nucleic acid sequence to which a transcription factor can bind. Further preferably, the element comprises a nucleic acid sequence to which a transcription factor selected from, but not limited to, GC, GATA, Ets, Sp1, Egr1, NF-E2, WT-1, and AP1, can bind. Optionally, the element is selected from Table 2.

The at least one nucleic acid modification or substitution or the at least one further nucleic acid may be introduced at a location, such that the ability of a transcription factor to bind to the element, for example a transcription factor binding site, is altered. The ability of a transcription factor to bind to the element may be positively altered or may be negatively altered.

Optionally, the at least one nucleic acid modification or substitution or the at least one further nucleic acid may be introduced at a location, such that the ability of a transcription factor to bind to the element, for example a transcription factor binding site, is positively altered. By positively altered is meant that the element is altered such that a transcription factor is capable of binding to the element. Optionally, the element comprises a nucleic acid sequence to which a transcription factor can bind. Further optionally, the element comprises a nucleic acid sequence to which a transcription factor selected from, but not limited to, GC, GATA, Ets, Sp1, Egr1, NF-E2, WT-1, and AP1, can bind. Optionally, the element comprises a nucleic acid sequence selected from any one of SEQ ID NOs: 8, 10, 12, 14, 16, 18, 20, 22, 24, 26, 28, 30, 32, 34, and 36, as indicated under the Wild-type Sequence (5′ to 3′) column in the Table 1 herein.

Alternatively, the at least one nucleic acid modification or substitution or the at least one further nucleic acid may be introduced at a location, such that the ability of a transcription factor to bind to the element, for example a transcription factor binding site, is negatively altered. By negatively altered is meant that the element is altered such that a transcription factor is less, optionally not, capable of binding to the element, relative to the unmodified element. Optionally, the element comprises a nucleic acid sequence to which a transcription factor cannot bind. Further optionally, the element comprises a nucleic acid sequence to which a transcription factor selected from, but not limited to, GC, GATA, Ets, Sp1, Egr1, NF-E2, WT-1, and AP1, cannot bind. Optionally, the element comprises a nucleic acid sequence selected from any one of SEQ ID NOs: 9, 11, 13, 15, 17, 19, 21, 23, 25, 27, 29, 31, 33, 35, and 37, as indicated under the Mutated Sequence column in the Table 1 herein.

Optionally, the at least one nucleic acid modification or substitution or the at least one further nucleic acid is introduced at a location such that a transcription factor cannot bind to the location or the element. Optionally, the at least one nucleic acid modification or substitution is a deletion. Further optionally, the at least one nucleic acid modification or substitution is a nucleic acid substitution.

Optionally, the thromboxane A2 receptor is the human thromboxane A2 receptor. Optionally, the promoter is the promoter of an isoform of thromboxane A2 receptor. Preferably, the promoter is the promoter of the alpha isoform of thromboxane A2 receptor.

It is understood that promoter variants, or fragments or multimers thereof, comprising nucleic acid substitutions that preserve the structure and functional properties of the polynucleotides described herein fall within the scope of the present invention.

It is also understood that, polynucleotide variants or fragments thereof, which are at least 75%, optionally at least 85%, further optionally at least 90% homologous to the polynucleotides described herein fall within the scope of the present invention.

Optionally, the promoter sequence is at least 75%, optionally at least 85%, further optionally at least 90% homologous to the nucleic acid sequence defined in SEQ ID NO 1. As used herein, SEQ ID NO 1 refers to nucleotides −8500 to −5895 of human Prm1, when compared to the position with respect to ATG start site (at position +1) of the TP gene.

Optionally or additionally, the at least one location is further independently selected from one or more of the group comprising, but not limited to, locations whose 5′ most nucleotides are at or adjacent nucleic acid positions −6007, −6022, −6080, −6098, −6206, −6278, −6294, −6717, −7805, −7870, −7890, −7831, −8146, −8281 and −8345. All positions with respect to ATG start site (at +1) of the TP gene, where each actual nucleotide number indicated represents the 5′ nucleotide of the respective consensus transcription binding site of the wild type sequences (Table 1).

Further optionally or additionally, the at least one location is further independently selected from one or more of the group comprising, but not limited to, locations whose 5′ most nucleotides are at or adjacent nucleic acid positions −8500, −7962, −7717, −6848, and −6320.

Optionally, the gene transcription-regulating polynucleotide comprises one or both of: at least one nucleic acid sequence selected from the group comprising SEQ ID NOs 2-6; or at least one nucleic acid modification and/or substitution or at least one further nucleic acid introduced at least one location within SEQ ID NO 1 of the nucleic acid sequence of thromboxane A2 receptor promoter, the at least one location being independently selected from one or more of the group comprising, but not limited to, locations whose 5′ most nucleotides are at or adjacent nucleic acid positions −6007, −6022, −6080, −6098, −6206, −6278, −6294, −6717, −7805, −7870, −7890, −7831, −8146, −8281 and −8345. All positions with respect to ATG start site (at +1) of the TP gene, where each actual nucleotide number indicated represents the 5′ nucleotide of the respective consensus transcription binding site of the wild type sequences (Table 1).

Optionally, the gene transcription-regulating polynucleotide further comprises at least one element comprising a nucleic acid sequence, which facilitates initiation of transcription. Preferably, the element is a cis-regulatory element. Optionally, the element comprises an RNA polymerase binding site, or a binding site for any member of the transcription preinitiation complex. Preferably, the element comprises a transcription-factor binding site. The transcription factor binding site preferably binds a transcription factor, which facilitates initiation of transcription. The transcription factor may facilitate binding of RNA polymerase. For example, the element may comprise the nucleic acid sequence TATA(A/T)A(A/T), or TATA box, which binds TATA binding protein (TBP, a subunit of TFIID). TBP, along with a variety of TBP-associated factors, make up the TFIID, a general transcription factor that in turn makes up part of the RNA polymerase II preinitiation complex, thereby facilitating initiation of transcription.

Optionally, the element comprises the nucleic acid sequence defined in SEQ ID NO 7 (positions −5895 to −6320), or fragment thereof. Alternatively, the element may comprise the nucleic acid sequence of the human cytomegalovirus (CMV) immediate-early enhancer and promoter.

Optionally, the polynucleotide comprises:
the nucleic acid sequence of SEQ ID NO 7 (positions −5895 to −6320), and
at least one nucleic acid sequence selected from the group comprising SEQ ID NOs 2-6; or
at least one nucleic acid modification and/or substitution or at least one further nucleic acid introduced at least one location within SEQ ID NO 1 of the nucleic acid sequence of thromboxane A2 receptor promoter, the at least one location being independently selected from one or more of the group comprising, but not limited to, locations whose 5′ most nucleotides are at or adjacent nucleic acid positions −6007, −6022, −6080, −6098, −6206, −6278, −6294, −6717, −7805, −7870, −7890, −7831, −8146, −8281 and −8345. All positions with respect to ATG start site (at +1) of the TP gene, where each actual nucleotide number indicated represents the 5′ nucleotide of the respective consensus transcription binding site of the wild type sequences (Table 1).

Optionally, the polynucleotide positively regulates gene transcription. By “positively regulates” is meant increased transcription of the gene above that found in a normal cellular state. Positive regulation may manifest as increased rates of transcription, or in an increased abundance of gene product relative to that found in a normal cellular state. Optionally, the polynucleotide comprises at least one element that can positively regulate gene transcription, and is described herein after as an activating sequence. Preferably, a first activating sequence comprises the nucleic acid sequence of SEQ ID NO 2 (positions −7962 to −7717), or a second activating sequence comprises the nucleic acid sequence of SEQ ID NO 3 (positions −7717 to −7504). All nucleotide sequence position numbers are based on intact Prm1.

Optionally, the polynucleotide negatively regulates gene transcription. By “negatively regulate” is meant reduced transcription of the gene below that found in a normal cellular state. Negative regulation may manifest as reduced rates of transcription, or in a reduced abundance of gene product relative to that found in a normal cellular state. Optionally, the polynucleotide comprises at least one element that can negatively regulate gene transcription, and is described herein after as a repressor sequence. Preferably, a first repressor sequence comprises the nucleic acid sequence of SEQ ID NO 4 (positions −8500 to −7962), or a second repressor sequence comprises the nucleic acid sequence of SEQ ID NO 5 (positions −6848 to −6648). All nucleotide sequence position numbers are based on intact Prm1. A third repressor sequence, designated RR3, is located between −6258 and −6123 within the proximal core promoter; SEQ ID NO 6 (positions −6258 to −6123).

The polynucleotide may include one or both activating sequences, whether modified or unmodified. The polynucleotide may include one, any two, or all repressor sequences, whether modified or unmodified. The polynucleotide may include a combination of at least one activating sequence, and at least one repressor sequence, whether modified or unmodified. Optionally, the polynucleotide may include a combination of at least one activating sequence, and at least one repressor sequence, either or both of which can be modified or unmodified. For example, it is envisaged that a polynucleotide comprising an unmodified activating sequence will positively regulate gene transcription, and that a polynucleotide comprising an activating sequence modified as described herein will negatively regulate gene transcription. Further, it is envisaged that a polynucleotide comprising an unmodified repressor sequence will negatively regulate gene transcription, and that a polynucleotide comprising a repressor sequence modified as described herein will positively regulate gene transcription.

Optionally, the polynucleotide comprises at least one further nucleic acid modification or substitution. Optionally, the at least one further nucleic acid modification or substitution is selected from the group comprising, but not limited to, a nucleic acid substitution at one or more locations whose 5′ most nucleotides are at or adjacent positions −6007, −6022, −6080, −6098, −6206, −6278, −6294, −6717, −7805, −7870, −7890, −7831, −8146, −8281 and −8345. All positions with respect to ATG start site (at +1) of the TP gene, where each actual nucleotide number indicated represents the 5′ nucleotide of the respective consensus transcription binding site of the wild type sequences (Table 1).

Further optionally, the at least one further nucleic acid modification or substitution is selected from the group comprising, but not limited to those indicated under the mutated sequence column in the Table 1 below:

| | Position with | | | | |
|---|---|---|---|---|---|
| | respect to | | | | |
| | ATG start site | Wild-type | SEQ | | SEQ |
| Binding | (@ + 1) of | Sequence* | ID | Mutated | ID |
| Site | the TP gene*** | (5′ to 3′) | NO | Sequence** | NO |
| GC | −8345 | tgccccCGCCcccac | 8 | tgccccTGACcccac | 9 |
| GC | −8281 | gcccgGCCCccgccgga | 10 | gcccgGTTCccgccgga | 11 |
| GC | −8146 | cGGGGGGTgggGGGCG | 12 | cGGGGGTCgtgGGGTG | 13 |
| | | GGGGGCgggccaa | | GATGGCgggccaa | |
| GC | −7831 | tcactGCCCcctcatct | 14 | tcactGTCCtctcatct | 15 |
| GATA | −7890 | cttgtTATCtcag | 16 | cttggTAGCtcag | 17 |
| Ets | −7870 | gacagAGGAagtgggga | 18 | gacagGTGAagtgggga | 19 |
| Ets | −7805 | gccccacaTCCTcctcc | 20 | gccccacaTCACcctcc | 21 |
| GC | −6717 | tctgtcctCCCAcccca | 22 | tctgtcctATCAcccca | 23 |
| Sp1/Egr1 | −6294 | cgaggGGCGtggcca | 24 | cgaggAACAtggcca | 25 |
| Sp1/Egr1 | −6278 | cgcagggtGGGCggggctg | 26 | cgcagggtGTATggggctg | 27 |
| GC | −6206 | cagcggccCCCAcccgt | 28 | cagcggccTACAcccgt | 29 |
| Sp1/Egr1 | −6098 | tgggcccGCCCctgg | 30 | tgggcccAATCctgg | 31 |
| NF- | −6080 | ccagaCTGActcagtttccct | 32 | ccagaCACActcagtttccct | 33 |
| E2/AP1 | | | | | |
| Sp1/Egr1 | −6022 | tctgcccGCCCccagccct | 34 | tctgcccTAACccagccct | 35 |
| Sp1/Egr1 | −6007 | ccctcgcccCACCctcgg | 36 | ccctcgcaaTACCctcgg | 37 |

Note:
*Core sequences of binding sites are in capital letters, while **mutated nucleotides are shaded/highlighted in yellow and underlined.
*Sequences given for all wild type sequences are those of the + strand of the TP gene.
***Nucleotide numbers indicated represent the 5′ nucleotide of each consensus element of the wild type sequences.

Optionally, the at least one further nucleic acid modification or substitution comprises a nucleic acid substitution at one or more locations whose 5′ most nucleotides are at or adjacent positions: −8345, −8281, and −8146. Further optionally, the at least one further nucleic acid modification or substitution comprises a nucleic acid substitution at one or more locations whose 5′ most nucleotides are at or adjacent position −6717.

Optionally, the at least one further nucleic acid modification or substitution comprises a nucleic acid substitution at one or more locations whose 5′ most nucleotides are at or adjacent positions: −7805, −7870, −7890, and −7831.

Preferably, the polynucleotide comprises the nucleic acid sequence defined in SEQ ID NO:1 and a nucleic acid modification and/or substitution at position −8146. Further preferably, the nucleic acid modification and/or substitution comprises a nucleic acid substitution. Still further preferably, the nucleic acid modification and/or substitution to the nucleic acid sequence defined by SEQ ID NO:13.

Alternatively, the polynucleotide comprises the nucleic acid sequence defined by nucleotide positions −7962 to −5895 of SEQ ID NO:1.

Further alternatively, the polynucleotide comprises the nucleic acid sequence defined by nucleotide positions −6848 to −5895 of SEQ ID NO:1. Optionally or additionally, the polynucleotide further comprises at least one nucleic acid modification and/or substitution selected from a nucleic acid modification/or substitution at a position selected from nucleic acid positions −6717, −6206, and −6800. Optionally, the nucleic acid modification or substitution selected comprises a nucleic acid substitution at a location selected from nucleic acid positions −6717, −6206, and −6800. Optionally, the nucleic acid substitution is selected from a G→C nucleic acid substitution at position −6800, CC→AT nucleic acid substitution at position −6717, and a CC→TA nucleic acid substitution at position −6206.

The present invention thereby provides the use of a gene transcription-regulating polynucleotide, wherein the polynucleotide comprises one or both of:
at least one element having a nucleic acid sequence selected from the group comprising SEQ ID NOs 2-6; or
at least one nucleic acid modification and/or substitution or at least one further nucleic acid is introduced at least one location within SEQ ID NO 1 of the nucleic acid sequence of thromboxane A2 receptor promoter, the at least one location being independently selected from one or more of the group comprising, but not limited to, locations whose 5′ most nucleotides are at or adjacent nucleic acid positions −6007, −6022, −6080, −6098, −6206, −6278, −6294, −6717, −7805, −7870, −7890, −7831, −8146, −8281 and −8345 to drive the expression of a gene. All positions with respect to ATG start site (at +1) of the TP gene, where each actual nucleotide number indicated represents the 5′ nucleotide of the respective consensus transcription binding site of the wild type sequences (Table 1).

Optionally, the polynucleotide comprises:
the nucleic acid sequence of SEQ ID NO 7 (positions −5895 to −6320), and
at least one nucleic acid sequence selected from the group comprising SEQ ID NOs 2-6; or
at least one nucleic acid modification and/or substitution or at least one further nucleic acid introduced at least one location within SEQ ID NO 1 of the nucleic acid sequence of thromboxane A2 receptor promoter, the at least one location being independently selected from one or more of the group comprising, but not limited to, locations whose 5′ most nucleotides are at or adjacent nucleic acid positions −6007, −6022, −6080, −6098, −6206, −6278, −6294, −6717, −7805, −7870, −7890, −7831, −8146, −8281 and −8345. All positions with respect to ATG start site (at +1) of the TP gene, where each actual nucleotide number indicated represents the 5′ nucleotide of the respective consensus transcription binding site of the wild type sequences (Table 1).

According to a further aspect of the present invention there is provided a method for regulating transcription of a gene, the method comprising providing a gene transcription-regulating polynucleotide in operable association with the gene, optionally within a host cell, wherein the gene transcription-regulating polynucleotide comprises the nucleic acid sequence of SEQ ID NO:1 of the nucleic acid sequence of thromboxane A2 receptor promoter or a fragment thereof, the gene transcription-regulating polynucleotide or the fragment thereof further comprising at least one nucleic acid modification and/or substitution. Preferably, the gene is in operable association with a polynucleotide of the present invention. Preferably, the gene is a heterologous gene. Optionally, the heterologous gene is a tissue factor gene. Further optionally, the heterologous gene is human tissue factor gene. The gene transcription-regulating polynucleotide can be in operable association with the gene within a host cell, which is stably or transiently transfected using a recombinant expression vector comprising a nucleic acid sequence of a polynucleotide of the present invention.

According to a further aspect of the present invention there is provided a recombinant expression vector comprising a nucleic acid sequence of a polynucleotide of the present invention. Preferably, the polynucleotide is in operable association with a gene of interest. Preferably, the recombinant expression vector can replicate or be maintained within a host cell. Preferably, the gene is in operable association with a polynucleotide of the present invention. Preferably, the gene is a heterologous gene. Optionally, the heterologous gene is a tissue factor gene. Further optionally, the heterologous gene is human tissue factor gene.

According to a still further aspect of the present invention there is provided a host cell, which is stably or transiently transfected using the recombinant expression vector of the present invention. Optionally, the host cell is selected from the group comprising, but not limited to, erythroleukemia cells, endothelial cells, embryonic kidney cells, smooth muscle cells, and fibroblast cells. Further optionally, the host cell is selected from the group comprising, but not limited to, human erythroleukemia cells, human endothelial cells, human embryonic kidney cells, human aortic smooth muscle cells, and human lung fibroblast cells.

According to a still further aspect of the present invention there is provided a method of diagnosing a disorder caused by, or associated with, dysregulated thromboxane A2 signalling, the method comprising the steps of identifying a nucleic acid modification and/or substitution within SEQ ID NO 1 of the nucleic acid sequence of the promoter of thromboxane A2 receptor, and associating the presence of the nucleic acid modification or substitution with a disorder caused by, or associated with, dysregulated thromboxane A2 signalling.

The nucleic acid modification is selected from the group comprising, but not limited to, a multiplication of the element, a deletion of the element, inversion of the element, and a nucleic acid substitution within the element.

Optionally, the thromboxane A2 receptor is the human thromboxane A2 receptor. Optionally, the promoter is the promoter of an isoform of thromboxane A2 receptor. Preferably, the promoter is the promoter of the alpha isoform of thromboxane A2 receptor. By “dysregulated” is meant any disturbance resulting in the abnormal functioning of a process, whereby the process no longer follows a conventional functional pattern associated with a normal cellular state. Dysregulated thromboxane A2 signalling may be caused by dysregulated receptor gene transcription, dysregulated receptor gene translation, or dysregulated receptor function, each of which may be attributable to a nucleic acid modification or substitution. Disorders may be, for example, vascular disorders, such as thrombosis, unstable coronary artery disease, ischaemic heart disease, congestive heart failure; neoplastic disorders, such as paediatric kidney cancer, breast cancer, oesophageal cancer, and pancreatic cancer; preterm labour, pre-eclampsia, and renal disorders, such as inflammatory renal disease, adult renal syndrome, diabetic nephropathy and renal failure.

According to a still further aspect of the present invention there is provided a method for treating a patient suffering from a disorder caused by, or associated with, dysregulated thromboxane A2 signalling, the method comprising the step of either rendering Prm1 non-functional; or rendering genetically mutated Prm1 functionally normal with respect to the pattern of Prm1 transcription in human cells and tissues and/or the quantification of expression to reflect that found in normal cells or tissues.

Optionally, the method comprises the step of introducing a nucleic acid modification and/or substitution within an element of the nucleic acid sequence of the promoter of thromboxane A2 receptor. Optionally, the method comprises the step of removing an element from the nucleic acid sequence of the promoter of thromboxane A2 receptor. Optionally, the method may be carried out in situ. Further optionally, the method may be carried out ex vivo or in vitro. It is understood that an in situ procedure involves carrying out the method internal to the patient. An ex vivo procedure involves carrying out the method external to the patient, and, optionally, further comprises the step of reintroducing the non-functional element into the patient.

Optionally, the method comprises the inhibition or restoration of transcription factor binding to at least one location or element. Optionally, the method comprises the inhibition or restoration of transcription factor binding to the promoter. Optionally, transcription factor binding is achieved or impeded by chemical means, or by physical means. In the case of chemical means, a chemical substance may be used to alter the chemical interaction between the transcription factor and the element. In the case of physical means, a substance, which may be chemical or biological in nature, may be used to compete with transcription factor binding to the element. Optionally, cooperative transcription factor interaction to form dimers or oligomers followed by binding is achieved or impeded by chemical means, or by physical means. In the case of chemical means, a chemical substance may be used to alter the chemical interaction between the transcription factor and the element. In the case of physical means, a substance, which may be chemical or biological in nature, may be used to compete with transcription factor binding to the element. Optionally, the transcription factor may be altered or modified to inhibit or restore binding to the element.

For the purposes of the present specification, it is understood that this invention is not limited to the specific methods, treatment regimens, or particular procedures, which as such may vary. Moreover, the terminology used herein is for the purpose of describing particular embodiments and is not intended to be limiting.

### BRIEF DESCRIPTION OF THE DRAWINGS

FIG. 1: Organisation of the human TP gene. Panel A: The human TP gene contains 3 exons (E) 1, E2 and E3 separated by 2 introns (I) 1 and I2. An additional exon, E1b, is located within I1 and there are 2 putative promoters (P) 1 and P2, located 5′ of E1 and E1b sequences, respectively. The lower numbering system indicates the position of those sequences within the TP gene, spanning from −8500 to +6547 (italics, underlined) while the upper numbering system indicates the position of the exon sequences within the TP mRNA(s). All nucleotide numbers are assigned relative to the translation start site, ATG designated +1 and all sequences 5′ of +1 are given a − designation and all numbers 3′ of +1 are given a + designation. E1, encodes nucleotides −289 to −84 of 5′ untranslated region (UTR) of the TP mRNA; alternatively, exon E1b, of 115 bp, located within I1, encodes −199 to −84 of 5′UTR sequence. E2 contains nucleotides −83 to −1 of 5′ UTR sequence and +1 to +786 of coding sequence, encoding amino acids 1-261. E3 contains nucleotides +787 to +1029, coding for amino acids 262-343 of TPα, and nucleotides +1030 to +1938, representing 3′ UTR sequences Nucleotides +984 to +1642 behave as a potential intron (Intron 3b) on the TP mRNA; splicing of nucleotides +983/+1643 generates a mRNA which has a novel open reading frame, encoding TPα of 407 amino acids, whereby nucleotides +983 to +1221 encode amino acids 328-407 that are unique to TPα.

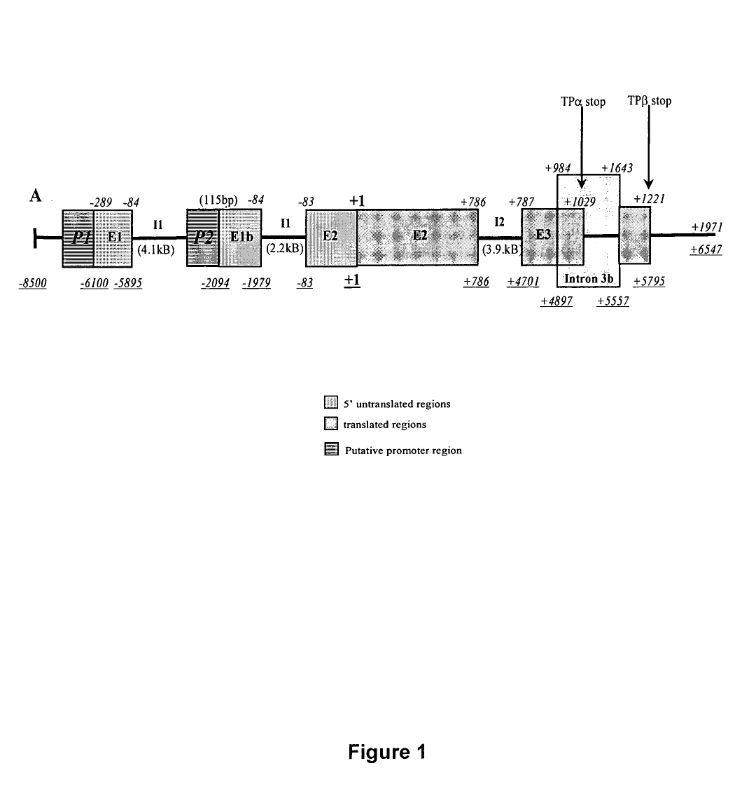

FIG. 2: Effect of 5′ and 3′ Deletions on Prm1-directed Gene Expression. Schematic of the human TP gene spanning nucleotides −8500 to +786, encoding Prm1 (−8500 to −5895), Prm3, exon (E)1, intron (I)1 and E2, where nucleotide +1 represents the translational start site (ATG). Plasmids (2 μg) encoding: Panel A: pGL3control (positive control; 23.9±1.1 RLU), Prm1, Prm1B, Prm1BΔ, Prm1C, Prm1D, Prm1E; Panel B: Prm1BΔ, Prm1BΔ 3′deletion, Prm1C, Prm1C 3′deletion, Prm1E, Prm1E 3′deletion or, as a negative control, pGL3Basic (A & B) were co-transfected with pRL-TK into HEL 92.1.7 cells. Mean firefly relative to renilla luciferase activity was expressed in arbitrary relative luciferase units (RLU±SEM; n=5).

FIG. 3: Identification of NF-E2/AP1 and Sp1/Egr1 Elements within Prm1. Putative Sp1/Egr1 and NF-E2/AP1 elements within Prm1, where the 5′ nucleotide is indicated and the star symbol signifies mutated elements. pGL3Basic plasmids (2 μg) encoding: Panel A: Prm1E, Prm1HΔ, Prm1K, Prm1L, Prm1L^{Sp1/Egr1(-6007)}* and, as a control, pGL3Basic or Panel B: Prm1HΔ, Prm1HΔ^{Sp1/Egr1(-6294)}*, Prm1HΔ^{Sp1/Egr1(-6278)}*, Prm1HΔ^{Sp1/Egr1(-6098)}*, Prm1HΔ^{NF-E2/AP1(-6080)}*, Prm1HΔ^{Sp1/Egr1(-6022)}*, Prm1HΔ^{Sp1/Egr1(-6007)}*, or Panel C: Prm1HΔ, Prm1HΔ^{Sp1/Egr1(-6294,-6022)}*, Prm1HΔ^{Sp1/Egr1(-6022,-6007)}*, were co-transfected with pRL-TK into HEL 92.1.7 cells. Luciferase activity was expressed as mean firefly relative to renilla luciferase activity (RLU±SEM; n=5).

FIG. 4: NF-E2 Binding to the Proximal Prm1. EMSAs (Panel A) or supershift assays (Panel B) using nuclear extract from HEL cells and a biotin-labelled double-stranded NF-E2/AP1 probe (Probe) spanning −6087 to −6049 of the TP gene, as indicated by the horizontal bar. Panel A: Nuclear extract was pre-incubated with the vehicle (−) or with excess non-labelled competitor oligonucleotides (+) prior to addition of the NF-E2/AP1 probe. One main complex C1, as well as one or more faster-migrating complexes, were observed; prolonged exposure revealed a slower-migrating C2 complex (not shown). Panel B: Nuclear extract was pre-incubated with vehicle (−), anti-NF-E2 (+), or anti-cJun (+) sera before addition of the biotinylated NF-E2 probe. Two main complexes, C1 and C2, were observed. The arrow to the right indicates the supershifted transcription factor:DNA complex detected with the anti-NF-E2 serum (lane 3). Panel C: Immunoblot analysis of NF-E2 expression in HEL cells (60 □g whole cell protein). Panels D & E: ChIP analysis of Prm1. Schematic of Prm1 and primers (arrows) used in the PCR to detect the proximal Prm1 region (−6368 to −5895; Panel D) from input chromatin or anti-NF-E2 or, as a control, normal rabbit IgG immunoprecipitates of crosslinked chromatin from HEL cells. Primers to detect an upstream region of Prm1 (−8460 to −8006; Panel E) from input chromatin, anti-NF-E2 or normal rabbit IgG precipitates were used as a negative control. Images are representative of three independent experiments.

FIG. 5: Nuclear Factor Binding to Overlapping Sp1/Egr1 Sites within Prm1. Immunoblot analysis of Sp1 (Panel A) and Egr1 (Panel B) expression in HEL cells (80 μg whole cell protein/lane). EMSAs (Panel C) or supershift assays (Panel D) using nuclear extract from HEL cells and a biotinylated double-stranded Sp1/Egr1⁻⁶²⁹⁴ probe (Probe) spanning −6299 to −6276 of the TP gene, as indicated by the horizontal bar. Panel C: Nuclear extract was pre-incubated with the vehicle (−) or excess non-labelled competitor oligonucleotides (+) before addition of the Sp1/Egr1⁻⁶²⁹⁴ probe. Two main complexes, C1 and C2, were observed. Panel D: Nuclear extract was pre-incubated with vehicle (−) or with (+) anti-Sp1, anti-Egr1, anti-WT-1 or anti-cJun sera before addition of the Sp1/Egr1⁻⁶²⁹⁴ probe. Two main complexes were observed. EMSAs (Panel E) or supershift assays (Panel F) using nuclear extract from HEL cells and a biotinylated double-stranded Sp1/Egr1⁻⁶²⁷⁸ probe (Probe) spanning −6283 to −6255 of the TP gene, as indicated by the horizontal bar. Panel E: Nuclear extract was pre-incubated with the vehicle (−) or excess non-labelled competitor oligonucleotides (+) before addition of the Sp1/Egr1⁻⁶²⁷⁸ probe. One main complex, C1, was observed. Panel F: Nuclear extract was pre-incubated with vehicle (−) or with (+) anti-Sp1, anti-Egr1, anti-WT-1 or anti-cJun sera before addition of the Sp1/Egr1⁻⁶²⁹⁴ probe. One main complex, C1, was observed. Images are representative of three independent experiments.

FIG. 6: EMSA and ChIP analysis of Sp1 and Egr1 Binding to Prm1. EMSAs (Panel A) or supershift assays (Panel B) using nuclear extract from HEL cells and a biotinylated double-stranded Sp1/Egr1^{-6022,-6007} probe (Probe) spanning −6027 to −5985 of the TP gene, indicated by the horizontal bar. Panel A: Nuclear extract was pre-incubated with vehicle (−) or excess non-labelled competitor oligonucleotides (+) before addition of the Sp1/Egr1^{-6022,-6007} probe. Two main complexes, C1 and C2, were observed. Panel B: Nuclear extract was pre-incubated with vehicle (−) or with (+) anti-Sp1, anti-Egr1, anti-WT-1 or anti-cJun sera before addition of the Sp1/Egr1^{-6022,-6007} probe. The image on the right represents a longer exposure of the upper section of the same chromatogram on the left. The arrows indicate the supershifted transcription factor:DNA complex detected with the anti-Sp1 (lane 3, left panel) and anti-Egr1 (lane 4, right panel) sera, respectively. Panels C & D: ChIP analysis of Prm1. Schematic of Prm1 and primers (arrows) used in the PCR to detect the proximal Prm1 region (−6368 to −5895; Panel C) from input chromatin or immunoprecipitated crosslinked chromatin from HEL cells using anti-Sp1, anti-Egr1 or normal rabbit IgG sera. Primers to detect an upstream region of Prm1 (−8460 to −8006; Panel D) from input chromatin, anti-Sp1, anti-Egr1 or normal rabbit IgG precipitates were used as a negative control. Images are representative of three independent experiments. Panel E: Effect of over-expression of Egr1 on Prm1-directed gene expression. HEL cells were transiently co-transfected with pGL3b:Prm1HΔ plus pRL-TK in the presence of pCMV-Egr1 (+Egr1) or with pCMV5 (Control) and expressed as mean relative firefly to renilla luciferase activity (RLU±SEM; n=19).

FIG. 7: Identification of Functional GATA and Ets Elements within Prm1. The positions of putative GATA and Ets elements within Prm1, where the 5′ nucleotide of each element is shown and the star symbol signifies mutated elements. Recombinant pGL3Basic plasmids (2 μg) encoding: Panel A: pGL3control (positive control), Prm1B, Prm1BΔGata/Ets, Prm1BΔ or Panel B: Prm1Δ, Prm1B^{GATA(-7890)}*, Prm1B^{Ets(-7870)}*, Prm1B^{GATA (-7890)}*^{,Ets(-7870)}*, Prm1BΔGata/Ets, Prm1B□Gata/Ets^{Ets (-7805)}*and Prm1BΔ were co-transfected with pRL-TK into HEL cells. Panel C: A 245 bp subfragment of Prm1, spanning nucleotides −7962 to −7718, encoding either the wild type or mutated (*) GATA⁽⁻⁷⁸⁹⁰⁾ and Ets⁽⁻⁷⁸⁷⁰⁾ elements was subcloned into pGL3control vector upstream of the SV40 promoter. Resulting recombinant plasmids (0.5 μg), as well as pGL3control (0.5 μg), were co-transfected with pRL-TK into HEL cells. Luciferase activity was expressed as mean firefly relative to renilla luciferase activity (RLU±SEM; n=4).

FIG. 8: GATA-1 and Ets-1 Binding to Prm1. Panel A: EMSAs using nuclear extract from HEL cells and a biotinylated double-stranded GATA,Ets probe (Probe) spanning −7890 to −7848 of the TP gene, indicated by the horizontal bar. Nuclear extract was pre-incubated with vehicle (−) or excess non-labelled competitor oligonucleotides (+) before addition of the GATA/Ets probe. Four complexes, C1-C4, were observed. The image is representative of three independent experiments. Panels B & C: Immunoblot analysis of GATA-1 (50 kDa) and Ets-1 (54 kDa) expression in HEL cells (100 μg per lane). Panels D & E: ChIP analysis of Prm1. Schematic of Prm1 and primers (arrows) used in the PCR to detect the Prm1 region from −7978 to −7607 (Panel D) from input chromatin, anti-GATA-1, anti-Ets-1, or as a control, normal rabbit IgG immunoprecipitates, as indicated. Primers to detect the proximal Prm1 (−6368 to −5895; Panel E) from input chromatin, anti-GATA-1, anti-Ets-1 or normal rabbit IgG precipitates were used as a negative control. Images are representative of three independent experiments.

FIG. 9: Effect of 5′ deletions on Prm1-directed gene expression and identification of GC elements within the −8500 to −7962 region of Prm1. Panel A: Schematic of the human TP gene spanning nucleotides −8500 to +786 encoding Prm1 (−8500 to −5895), Prm3, exon (E)1, intron (I)1 and E2, where nucleotide +1 represents A of the translational start site (ATG) and nucleotides 5′ of that are given a − designation. pGL3Basic plasmids encoding Prm1 (−8500 to −5895) and its 5′ deletion fragments Prm1B (−7962), Prm1BΔGata/Ets (−7859), Prm1BΔ (−7717), Prm1C (−7504), Prm1D (−6848), Prm1E (−6648), Prm1I (−6258), Prm1J (−6123) and, as a control, pGL3Basic were co-transfected with pRL-TK into HEL 92.1.7 cells. Mean firefly relative to renilla luciferase activity was expressed in arbitrary relative luciferase units (RLU±SEM; n=6). Panels B, C and D: GC elements containing putative overlapping WT1/Egr1/Sp1 binding sites within Prm1, where the 5′ nucleotide is indicated and the star symbol signifies mutated elements. pGL3Basic plasmids encoding (B) Prm1, Prm1^{GC}*⁽⁻⁸³⁴⁵⁾, Prm1^{GC}*⁽⁻⁸²⁸¹⁾, Prm1^{GC}*⁽⁻⁸¹⁴⁶⁾, Prm1^{GC}*⁽⁻⁷⁸³¹⁾ and Prm1B or (C) Prm1, Prm1^{GC}*⁽⁻⁸³⁴⁵⁾, Prm1^{GC}*^{(-8345,-7831)}, Prm1^{GC}*^{(-8345,-8281,-7831)} and Prm1^{GC}*^{(-8345,-8281,-8146,-7831)} or (D) Prm1B, Prm1B^{GC}*⁽⁻⁷⁸³¹⁾ and Prm1B□ were co-transfected with pRL-TK into HEL cells. Luciferase activity was expressed as mean firefly relative to renilla luciferase activity (RLU±SEM; n=8).

FIG. 10: Expression of WT1, Sp1 and Egr1 proteins in HEL 92.1.7 cells. Western blot analysis of WT1 (Panel A), Egr1 (Panel B) and Sp1 (Panel C) expression in HEL cells (60 μg whole cell protein per lane). The positions of the molecular size markers (kDa) are indicated to the left, while the sizes of WT1, Sp1 and Egr1 are indicated to the right of the panels, respectively.

FIG. 11: Nuclear factor binding to 5′ GC elements within Prm1 in vitro. EMSAs using nuclear extract from HEL cells and biotin-labelled double-stranded probes encoding GC⁻⁸³⁴⁵, GC⁻⁸²⁸¹ (Panel A), GC⁻⁸¹⁴⁶ (Panel B) and GC⁻⁷⁸³¹ (Panel C). In each case, the horizontal bar indicates the relative position of the probe within Prm1. Nuclear extract was pre-incubated with the vehicle (−) or with excess non-labelled competitor oligonucleotides (+) prior to addition of the relevant probe. Images are representative of three independent experiments.

FIG. 12: ChIP analysis of WT1, Sp1 and/or Egr1 binding to the 5′ region of Prm1 and effect of over-expression of WT1 on Prm1-directed luciferase expression. Panels A and B: ChIP analysis of WT1, Sp1 and/or Egr1 binding to Prm1 in HEL 92.1.7 cells. Schematic of Prm1 and primers (arrows) used in the PCR to detect the −8460 to −8006 (Panel A) or the −7978 to −7607 (Panel B) regions of Prm1 using either input chromatin or chromatin extracted from anti-WT1, anti-Egr1, anti-Sp1 or, as a control, normal rabbit IgG immunoprecipitates. Images are representative of three independent experiments. Panel C: Effect of over-expression of WT1 on Prm1-directed gene expression. HEL cells were transiently co-transfected with 0.5 μg of pcDNA3 (control) or 0.5 μg of recombinant pcDNA3 plasmids encoding (+/+), (+/−), (−/+) or (−/−) isoforms of WT1, along with pGL3b:Prm1 (1.5 μg) plus pRL-TK (200 ng). Luciferase activity was expressed as mean relative firefly to renilla luciferase activity (RLU±SEM; n=6). Panel D: Western blot analysis to confirm over-expression of WT1 in HEL cells following transfection with 0.5 μg of pcDNA3 (control) or 0.5 μg of recombinant pcDNA3 plasmids encoding (+/+), (+/−), (−/+) or (−/−) isoforms of WT1 (25 μg whole cell protein per lane). The size of WT1 isoforms are indicated to the right of the panel. Panel E: RT-PCR analysis using primers to amplify TPα and GAPDH sequences from total RNA isolated from HEL cells following transfection with 0.5 μg of pcDNA3 (control) or 0.5 μg of recombinant pcDNA3 plasmids encoding (+/−) or (−/−) isoforms of WT1.

FIG. 13: Identification of GC elements within Prm1 regions from −6848 to −6648 and −6258 to −6123. Panels A and B: Schematic of Prm1 (−6848 to −5895) and the relative positions of GC elements containing putative overlapping WT1/Egr1/Sp1 binding sites, where the 5′ nucleotide is indicated and the star symbol signifies mutated elements. pGL3Basic plasmids encoding (A) Prm1D (−6848), Prm1D^{GC}*⁽⁻⁶⁷¹⁷⁾, Prm1E (−6648), Prm1I (−6258), Prm1I^{GC}*⁽⁻⁶²⁰⁶⁾ or Prm1J (−6123) or (B) Prm1 D, Prm1D^{GC}*⁽⁻⁶⁷¹⁷⁾, Prm1D^{GC}*⁽⁻⁶²⁰⁶⁾, Prm1D^{GC}*^{(-6717,-6206)} or Prm1E were co-transfected with pRL-TK into HEL 92.1.7 cells. Luciferase activity was expressed as mean firefly relative to renilla luciferase activity (RLU±SEM; n=9).

FIG. 14: Nuclear factor binding to GC⁻⁶⁷¹⁷ and GC⁻⁶²⁰⁶ elements within Prm1 in vitro and in vivo. Panels A and C: EMSAs using nuclear extract from HEL cells and a biotinylated double-stranded probe encoding (Panel A) the Prm1 GC⁻⁶⁷¹⁷ element and (Panel C) the Prm1 GC⁻⁶²⁰⁶ element where, in each case, the location of the specific probe within Prm1 is indicated by the horizontal bar. Nuclear extract was pre-incubated with vehicle (−) or excess non-labelled competitor oligonucleotides (+) before addition of the probe. One complex, C1, was observed in each case. The images are representative of three independent experiments. Panels B and D: ChIP analysis and schematic of Prm1 and primers (arrows) used in PCR to amplify the −6848 to −6437 (Panel B) and the −6368 to −5895 (Panel D) regions of Prm1 from input chromatin or from chromatin extracted from anti-WT1, anti-Egr1, anti-Sp1 or, as a control, normal rabbit IgG immunoprecipitates, as indicated. The images are representative of three independent experiments.

FIG. 15: Proposed model for WT1-mediated repression of Prm1 in HEL 92.1.7 cells Panel A: Schematic representation of the relative positions of functional binding elements within Prm1, as well as binding of the basal transcription apparatus (BTA) to the transcription initiation (TI) site. Overlapping Sp1/Egr1 elements at −6294, −6278, −6022 and −6007, as well as an NF-E2 element at −6080, located within the “core” proximal promoter, direct efficient basal activity of Prm1 in megakaryoblastic HEL cells. Additionally, GATA-1 and Ets-1 bind elements at −7890 and −7870, respectively, within UAR1 to increase Prm1 activity in HEL cells. The data herein indicate that WT1 binds to GC elements within URR1, specifically at −8345, −8281 and −8146, as well as elements at −7831 within UAR1, −6717 within URR2 and −6206 within RR3, to repress Prm1 activity. Panels B, C, D and E: Proposed model for WT-mediated repression of Prm1 in HEL cells. It is suggested that WT1 overcomes competition from other factors, such as Egr1 and Sp1 by binding cooperatively to neighbouring GC elements at −8345, −8281, −8146 and −7831 and independently to GC elements at −6717 and −6206 to mediate repression of Prm1-directed transcription by the basal transcription apparatus in HEL cells (Panel B). Mutation of any of the upstream GC elements at −8345, −8281, −8146 and −7831 by SDM interferes with cooperation among WT1 proteins binding to these elements, thereby inhibiting WT1 binding and alleviating repression of Prm1. In the absence of repressor binding to the remaining intact sites, these elements may now have a higher affinity for activating factors (Panel C). Disruption of remaining upstream GC elements blocks the binding of activators and results in de-activation of the promoter (Panel D). Furthermore, mutation of GC elements at −6717 and −6206 in Prm1D (−6848) alleviates repression of Prm1 (Panel E).

FIG. 16: Effect of PMA on TPα mRNA expression in HEL 92.1.7 cells. Panels A & B: RT-PCR analysis of RNA isolated from HEL cells incubated with PMA (100 nM; 1-48 h; lanes 2-11), where cells incubated with the vehicle [v; 0.1% (v/v) dimethylsulfoxide (DMSO); 48 h; lane 1] served as a control. Primers were used to amplify TPα and GAPDH mRNA sequences. Panel B: Southern blot analysis of the RT-PCR products co-screened using 5′ biotin-labeled oligonucleotide probes specific for TPα and GAPDH mRNA sequences. The images are representative of four independent experiments.

FIG. 17: Effect of 5′ deletions on the PMA-mediated increase of Prm1-directed gene expression. Panels A and B: Schematic of the human TP gene spanning nucleotides −8500 to +786 encoding Prm1 (−8500 to −5895), Prm3, exon (E)1, intron (I)1 and E2, where nucleotide +1 represents A of the translational start site (ATG) and nucleotides 5′ of that are given a −designation. pGL3Basic plasmids encoding Prm1 (−8500 to −5895) and its 5′ deletion fragments Prm1B (−7962), Prm1C (−7504), Prm1D (−6848), Prm1E (−6648), Prm1F (−6552) and Prm1K (−6067) were co-transfected with pRL-TK into HEL 92.1.7 cells. Approximately 32 h post-transfection, cells were incubated with either vehicle [veh; 0.1% (v/v) DMSO] or PMA (PMA; 100 nM) for 16 h. Data are presented as (Panel A) mean firefly relative to renilla luciferase activity expressed in arbitrary relative luciferase units (RLU±SEM; n=4) or (Panel B) fold induction of mean luciferase activity in PMA-treated cells compared to vehicle-treated cells. The asterisks (*) indicate that incubation of HEL cells with PMA significantly increased luciferase expression in HEL cells, where * and **** indicate p <0.05 and p<0.0001, respectively.

FIG. 18: Identification of PMA-responsive elements within the Prm1 region from −8500 to −7504. Panels A, B, C and D: Schematic of GC elements containing putative overlapping WT1/Egr1/Sp1 binding sites within Prm1, where the 5′ nucleotide is indicated and the star symbol signifies mutated elements. pGL3Basic plasmids encoding Prm1, Prm1^{GC}*⁽⁻⁸³⁴⁵⁾, Prm1^{GC}*⁽⁻⁸²⁸¹⁾, Prm1^{GC}*⁽⁻⁸¹⁴⁶⁾, or Prm1^{GC}*⁽⁻⁷⁸³¹⁾ (Panels A and B) or Prm1, Prm1^{GC}*⁽⁻⁸³⁴⁵⁾, Prm1^{GC}*^{(-8345,-7831)}, Prm1^{GC}*^{(-8345,-8281,-7831)} or Prm1^{GC}*^{(-8345,-8281,-8146,-7831)} (Panels B and C) were co-transfected with pRL-TK into HEL 92.1.7 cells. Cells were incubated with vehicle [veh; 0.1% (v/v) DMSO] or PMA (PMA; 100 nM) for 16 h. Data are presented as (Panels A and C) mean firefly relative to renilla luciferase activity expressed in arbitrary relative luciferase units (RLU±SEM; n=4) or (Panels B and D) fold-induction of mean luciferase activity in PMA-treated cells compared to vehicle-treated cells. The asterisks (*) indicate that PMA significantly induced luciferase expression in HEL cells, where **** indicates p<0.0001.

FIG. 19: Identification of a PMA-responsive element within the Prm1 region from −7962 to −7717. Panels A and B: Schematic of GC elements containing putative overlapping WT1/Egr1/Sp1 binding sites within Prm1, where the 5′ nucleotide is indicated and the star symbol signifies mutated elements. pGL3Basic plasmids encoding Prm1B, Prm1B^{GC}*⁽⁻⁷⁸³¹⁾,or Prm1C were co-transfected with pRL-TK into HEL 92.1.7 cells. Cells were incubated with vehicle [veh; 0.1% (v/v) DMSO] or PMA (PMA; 100 nM) for 16 h. Data are presented as (Panel A) mean firefly relative to renilla luciferase activity expressed in arbitrary relative luciferase units (RLU±SEM; n=4) or (Panel B) fold induction of mean luciferase activity in PMA-incubated cells compared to vehicle-incubated cells. The asterisks (*) indicate that PMA significantly induced luciferase expression in HEL cells, where **, and **** indicate p<0.01 and p<0.0001, respectively.

FIG. 20: Effect of PMA on expression of WT1, Sp1 and Egr1 proteins in HEL 92.1.7 cells. Panels A-D: Immunoblot analysis of WT1, Egr1, Sp1 and HDJ2 expression, respectively, in HEL cells pre-incubated with vehicle [v; 0.1% (v/v) DMSO; 48 h; lane 1] or PMA (100 nM; 0-48 h; lanes 2-12). The sizes of WT1, Egr1, Sp1 and HDJ2 proteins are indicated to the left of the panels. The images are representative of four independent experiments.

FIG. 21: Effect of NAB1 over-expression and ERK 1/2 signaling on the PMA-mediated induction of Prm1-directed luciferase expression. Panels A and B: Effect of over-expression of NAB1 on Prm1-directed gene expression. HEL cells were transiently co-transfected with pCMV5 (control) or pCMV:NAB1 along with pGL3b:Prm1 plus pRL-TK. Approximately 32 h post-transfection, cells were incubated with vehicle [veh; 0.1% (v/v) DMSO] or PMA (PMA; 100 nM) for 16 h. Data are presented as (Panel A) mean firefly relative to renilla luciferase activity expressed in arbitrary relative luciferase units (RLU±SEM; n=3) or (Panel B) fold induction of mean luciferase activity in PMA-incubated cells compared to vehicle-incubated cells. The asterisks (*) indicate that PMA significantly induced Prm1-directed luciferase expression in HEL cells, where **** indicate p<0.0001 (Panel A), or that over-expression of NAB1 significantly reduced PMA-induction of Prm1-directed luciferase expression, where **** indicate p<0.0001 (Panel B). Panel C: Effect of PD98059 (MEK1 inhibitor, 2-(2-amino-3-methoxyphenyl)-4H-1-benzopyran-4-one) on the PMA-mediated induction of Prm1-directed gene expression. HEL cells were transiently co-transfected with pRL-TK plus pGL3b:Prm1 or pGL3b:Prm1K. Cells were incubated with vehicle [veh; 0.1% (v/v) DMSO], PMA (PMA; 100 nM) or 100 nM PMA plus 10 μM PD98059 (PMA+PD98059) for 16 h. Luciferase activity is expressed as mean firefly relative to renilla luciferase activity expressed in arbitrary relative luciferase units (RLU±SEM; n=3). Panel D: Effect of PD98059 on the PMA-mediated increase in Egr1 expression in HEL cells. Immunoblot analysis of Egr1 expression in HEL cells incubated for 16 h with: vehicle [0.1% (v/v) DMSO], 10 μM PD98059, 100 nM PMA or 100 nM PMA plus 10 μM PD98059, where non-stimulated HEL cells served as an additional control (0 h). The size of the Egr1 protein (approximately 82 kDa) is indicated to the left of the panel. The image is representative of three independent experiments.

FIG. 22: Time-course of PMA-mediated induction of Prm1-directed luciferase expression in HEL cells. HEL cells were transiently co-transfected with pGL3b:Prm1 plus pRL-TK and cells were incubated with vehicle [v; 0.1% (v/v) DMSO; 48 h] or PMA (100 nM; 0-48 h). Luciferase activity is expressed as mean firefly relative to renilla luciferase activity expressed in arbitrary relative luciferase units (RLU±SEM; n=4). The asterisks (*) indicate that PMA significantly induced Prm1-directed luciferase expression in HEL cells, where *, ***, and **** indicate p<0.01, p<0.001 and p<0.0001, respectively.

FIG. 23: Nuclear factor binding to elements within the −8460 to −8006 region of Prm1 in vivo and PMA-induced translocation of WT1 from the nucleus to the cytoplasm. Panels A and B: ChIP analysis of WT1, Sp1 and Egr1 protein binding to Prm1. Schematic of Prm1 and primers (arrows) used in PCR to detect the −8460 to −8006 (Panel A) and −6368 to −5895 (Panel B) regions of Prm1 from input chromatin or chromatin extracted from anti-WT1, anti-Egr1, and anti-Sp1 immunoprecipitates or, as a control, from a normal rabbit IgG precipitate, as indicated, from HEL cells that were non-treated (0 h), or treated with 100 nM PMA for 5, 8 or 16 h, as specified. Images are representative of three independent experiments. Panel C: Confocal microscopy of HEL cells that were pre-incubated with PMA for 0, 1, 5, 8, 16, or 24 h, followed by immunolabeling with anti-WT1 antibody and AlexaFluor488 conjugated anti-rabbit IgG (green), followed by counterstaining with DAPI (red). Co-localization was observed by merging the green and the red channels (yellow). Images are representative of three independent experiments.

FIG. 24: Effect of 1α, 25-dihydroxy-vitamin D₃ on Prm1-directed gene expression and Egr1 protein expression in HEL cells. Panel A: Effect of 1α, 25-dihydroxy-vitamin D₃ on Prm1-directed gene expression. HEL cells were transiently co-transfected with pRL-TK plus pGL3b:Prm1 or pGL3b:Prm1K. Cells were incubated with vehicle (H₂O; veh) for 30 h or with 80 nM Vitamin D₃ for 24 h (24 h) or 30 h (30 h). Luciferase activity was expressed as mean firefly relative to renilla luciferase activity expressed in arbitrary relative luciferase units (RLU±SEM; n=3). The asterisks (*) indicate that PMA significantly induced Prm1-directed luciferase expression in HEL cells, where * and *** indicate p<0.01 and p<0.001, respectively. Panel B: Effect of Vitamin D₃ on Egr1 expression in HEL cells. Immunoblot analysis (60 μg of total protein per lane) of Egr1 expression in HEL cells incubated for 72 h with: vehicle (H₂O; v) or Vitamin D₃ (80 nM; Vit. D₃), where non-stimulated HEL cells served as an additional control (0 h). The size of the Egr1 protein (approximately 82 kDa) is indicated to the left of the panel. The image is representative of three independent experiments.

FIG. 25: Proposed model for PMA-mediated increases in Prm1 activity Panel A: Schematic representation of the relative positions of functional binding elements within Prm1, as well as binding of the basal transcription apparatus (BTA) to the transcription initiation (TI) site. Overlapping Sp1/Egr1 elements at −6294, −6278, −6022 and −6007, as well as an NF-E2 element at −6080, located within the “core” proximal promoter, direct efficient basal activity of Prm1 in megakaryoblastic HEL cells. GATA-1 and Ets-1 bind elements at −7890 and −7870, respectively, within UAR1 to increase Prm1 activity in HEL cells. WT1 binds to GC elements within URR1, specifically at −8345, −8281 and −8146, as well as elements at −7831 within UAR1, −6717 within URR2 and −6206 within RR3, to repress Prm1 activity. Panels B, C, D and E: Proposed model for PMA-induction of Prm1 in HEL cells. In quiescent HEL cells, WT1 binds cooperatively to multiple neighbouring GC elements at −8345, −8281, −8146 and −7831 within Prm1 to repress transcription by impairing the initiation of transcription by the BTA at the transcription initiation (TI) site (represented by an arrow; Panel B). Following exposure of HEL cells to PMA for approximately 5 h, ERK-mediated up-regulation of Egr1 expression results in increased competition between Egr1 and WT1 for binding to 5′ GC elements. This leads to displacement of WT1 and increased Egr1 binding to the GC elements, thereby enhancing transcription initiation by the BTA and leading to the initial increase in Prm1 activity and TPα expression during the early stages of differentiation of HEL cells induced by PMA (Panel C). Following exposure of HEL cells to PMA for approximately 8 h, a more pronounced increase in Egr1 binding occurs, with an associated decrease in WT1 binding. It has been suggested that translocation of WT1 from the nucleus to the cytoplasm following PMA-stimulation of HEL cells is responsible for the decrease in WT1 binding. This leads to promoter de-repression and facilitates a further increase in Egr1 binding, leading to a more pronounced activation of Prm1 by the BTA (Panel D). Following exposure of HEL cells to PMA for approximately 16 h, increased Egr1 turnover leads to a decrease in Egr1 expression. It is also suggested that PMA-mediated differentiation of HEL cells leads to phosphorylation of Sp1, enhancing its DNA binding activity. Therefore, increased affinity of Sp1 for the 5′ GC elements, associated with increased turnover of Egr1, facilitates binding of Sp1 to Prm1, thereby mediating a sustained increase in Prm1 activity as differentiation of HEL cells progresses toward the platelet phenotype (Panel E).

FIG. 26: Modified Prm1 promoter-directed gene expression in a panel of different cell types. Plasmids, encoding 5′ deletion fragments of Prm1, pGL3B:Prm1, pGL3B:Prm1^{GC-8146}*, pGL3B:Prm1B, pGL3B:Prm1D, pGL3B:Prm1D^{WT1(a),(b)}* and, as controls, pGL3Control & pGL3Basic (empty vector) were co-transfected with pRL-TK, into HEL (Human erythroleukemia) 92.1.6 (A), EA.hy 926 (human endothelial) (B), and HEK (Human embryonic kidney) 293 (C), cells.

FIG. 27: Modified Prm1 promoter-directed gene expression in a panel of different cell types. Panels A & B: Plasmids, encoding 5′ deletion fragments of Prm1, pGL3B:Prm1, pGL3B:Prm1^{GC-8146}*, pGL3B:Prm1B, pGL3B:Prm1D, pGL3B: Prm1D^{WT1(a),(b)}* and, as controls, pGL3Control & pGL3Basic (empty vector) were co-transfected with pRL-TK, into 1° hAoSMC (primary human aortic smooth muscle) (A), and WI-38 (human lung fibroblast) (B) cells. Panel C: Investigation of the Prm1D derivatives (Prm1D, Prm1D^{WT1(a)}*^{(b)}*, and Prm1D^{WT1(a)}*^{(b)}*^{(X)}* to direct luciferase expression in HEL cells, where WT1^{(a)}* refers to the mutation of the WT1 site at −6717, WT1^{(b)}* refers to the mutation of the WT1 site at −6206 and WT1^{(X)}* refers to the mutation of a repressor site at −6800.

FIG. 28: Immunoblot analysis of human tissue factor expression in HEL cells. HEL cells were co-transfected with 2 μg pcDNA3.1(−), pcDNA3.1(−)-hTF, pPrm1^{GC,-8146}*:hTF, pPrm1B:hTF and pPrm1D^{WT1(a),(b)}* along with 200 ng pRL-TK. Cells were analysed 48 h post-transfection by western blotting (60 μg of whole cell protein per lane) along with a hTF Biomass control using anti-hTF (upper panel) antibody; to confirm equal protein loading, immunoblots were stripped and rescreened with an anti-HDJ-2 antibody (lower panel).

### MATERIALS AND METHODS

### Materials

pGL3Basic, pRL-Thymidine Kinase (pRL-TK), and Dual Luciferase® Reporter Assay System were obtained from Promega Corporation. DMRIE-C®, RPMI 1640 culture media and fetal bovine serum (FBS) were from Invitrogen Life Technologies. Anti-NF-E2 (sc-291×), anti-Sp1 (sc-59×), anti-Egr1 (sc-110×), anti-WT-1 (sc-192×), anti-cJun (sc-45×), anti-GATA-1 (sc-13053×), anti-Ets-1 (sc-350×), rabbit IgG (sc-2027), and goat anti-rabbit horseradish peroxidase (sc-2204) were obtained from Santa Cruz Biotechnology. Anti-HDJ2 antibody was obtained from Neomarkers. All antibodies used for ChIP analysis were ChIP-validated by the supplier (Santa Cruz) and have been widely used in the literature for such analyses (Hoffmann et al., 2008; Kooren et al., 2007; Murakami et al., 2006; Sawado et al., 2001; Sinha-Datta et al., 2004; Sobue et al., 2005; Sohn et al., 2005; Zhang et al., 2002). The plasmid pCMV-Egr1 was kindly provided by Dr Gerald Thiel, University of Saarland Medical Centre, Homburg, Germany (Thiel et al., 1994). Bioinformatic analyses to identify putative transcription factor binding sites within Prm1 were carried out using the MatInspector™ programme (Quandt et al., 1995). Anti-human Tissue Factor (SC-20160) antibody was obtained from Santa Cruz. The plasmid pDNR-LIB: Tissue Factor (FactorIII/thromboplastin), containing a 1.374 kb insert encoding full length cDNA for Tissue Factor (GenomeCube#IRAUp969D0152D; Genbank number BC011029; CDS=169-1056(887 bp)), was purchased from Genome Cube.

### Construction of Luciferase-Based Genetic Reporter Plasmids

Promoter (Prm)1 is defined as nucleotide positions −8500 to −5895, located upstream (5′ flanking) of A of the translational ATG initiation codon, designated +1. The plasmid pGL3b:Prm1, containing the Prm1 sequence (2605 bp) in the pGL3Basic genetic reporter vector, has been previously described (Coyle et al., 2002). To identify elements required for Prm1 activity, a series of 5′- and 3′-deletion subfragments were subcloned into pGL3Basic. The recombinant plasmids generated, as well as the identities, sequence and corresponding nucleotides of the specific primers used for each fragment are listed in the expanded Materials and Methods section below. The identity and fidelity of all recombinant plasmids was verified by DNA sequence analysis. Additional 5′ deletion sub-fragments of Prm1 were amplified by the polymerase chain reaction (PCR) using pGL3b:Prm1 as template and subsequently sub-cloned into pGL3Basic. Specifically, for pGL3b:Prm1I, a PCR fragment was generated using the sense primer Kin358 (5′GAGAGGTACCTGAGAGACAGCGGGAGACAGA GAC3′; nucleotides (nu) −6258 to −6235, SEQ ID NO:38, where the underlined sequence corresponds to a Kpn1 cloning site) and the antisense primer Kin109 (5′AGAGACGCGTCTTCAGAGA CCTCATCTGCGGGG3′; complementary to nu −5917 to −5895 of Prm1, SEQ ID NO: 39, where the underlined sequence corresponds to a Mlu1 cloning site). For pGL3b:Prm1J, a fragment was generated using sense primer Kin391 (5′GAGAGGTACCCCTCCATCTGTGTGG GTCCTC 3′; nu −6122 to −6102, SEQ ID NO: 40) and the antisense primer Kin109. The identity and fidelity of all Prm1-derived sub-fragments in the corresponding recombinant pGL3Basic plasmids were verified through DNA sequencing. An additional 5′ sub-fragment of Prm1, termed Prm1F, was amplified by the polymerase chain reaction (PCR) using pGL3b:Prm1 as template and subsequently sub-cloned into the Kpn1-Mlu1 sites of pGL3Basic. Specifically, Prm1F was generated using the sense primer Kin235 (5′GAGAGGTACCTCCAGGCCTTGGGTGCTG3′; nucleotides (nu)−6552 to −6535, SEQ ID NO: 41, where the Kpn1 cloning site is underlined) and the antisense primer Kin109 (5′AGAGACGCGTCTTCAGAGACCTCATCTGCGGGG3′; complementary to nu −5917 to −5895 of Prm1, SEQ ID NO: 39, where the Mlu1 site is underlined). The identity and fidelity of all Prm1 gene fragments in the corresponding recombinant plasmids was verified through DNA sequencing. The full length coding sequence for human tissue factor (hTF) was amplified by PCR using pDNR-LIB/TF (IRAUp969D0152D GenomeCube ID) as template versus Kin623 (5′GAGAAAGCTTTTATGAAACATTCAGTGGGGAG-3′, SEQ ID NO: 42) and Kin624 (5′ GATGTTCCAGATTACGCTAGCCTCTGGGTCTGCTGCCTGTG-3′, SEQ ID NO: 43) and Pfu Turbo and the resulting 887 bp fragment subcloned into the BamHI/HindIII sites of the pcDNA3.1(−) vector to generate the recombinant plasmid pcDNA3.1(−):hTF. The plasmid pcDNA3.1(−):hTF was confirmed correct by restriction endonuclease digestion and was fully validated by DNA sequence analysis. The plasmid pPrm1^{GC-8146}:hTF encoding the full length coding sequence for hTF under the control of Prm1^{GC-8146} was generated by ligating a purified 5174 bp PciI to NheI fragment, encoding Prm1^{GC-8146} and generated from the plasmid pGL3B:Prm1GC⁻⁸¹⁴⁶, to a 4915 bp PciI to NheI fragment, encoding full length hTF and purified from pcDNA3.1(−)-hTF, to generate pPrm1^{GC-8146}:hTF. The plasmid pPrm1B:hTF encoding the full length coding sequence for hTF under the control of Prm1B was generated by ligating a purified 4636 bp PciI to NheI fragment, encoding Prm1B and generated from the plasmid pGL3B:Prm1B, to a 4915 bp PciI to NheI fragment, encoding full length hTF and purified from pcDNA3.1(−)-hTF, to generate pPrm1B:hTF. The plasmid pPrm1D^{WT1(a)}*^{,WT1(b)}*:hTF encoding the full length coding sequence for hTF under the control of Prm1D^{WT1(a)}*^{,WT1(b)}* was generated by ligating a purified 3522 bp PciI to NheI fragment, encoding pPrm1D^{WT1(a)}*^{,WT1(b)}* and generated from the plasmid pGL3B: pPrm1D^{WT1(a)}*^{,WT1(b)}*, to a 4915 bp PciI to NheI fragment, encoding full length hTF and purified from pcDNA3.1(−)-hTF, to generate pPrm1D^{WT1(a)}*^{,WT1(b)}*:hTF. Note; Prm1D^{WT1(a)}*^{,WT1(b)}* contains the mutated Wilms' tumor 1 sites at −6717 (WT1(a)*) and −6206 (WT(b)*) sites, respectively.

### Site-Directed Mutagenesis

Site-directed mutagenesis was carried out using the Quik-Change™ method (Stratagene).

The identities of the Prm1 elements subjected to site-directed mutagenesis and the corresponding plasmids generated, as well as the identity, sequence and corresponding nucleotides of the specific primers used are listed in the expanded Materials and Methods section below. The following lists the name and starting position of the Prm1 elements that were subjected to site-directed mutagenesis, the nucleotides that were changed, the templates that were used, the name of the corresponding plasmids generated, as well as the specific primers, their sequences and corresponding nucleotides. In each case, the − designation indicates nucleotides 5′ of the translational ATG start codon (designated +1).
1. GC at −8345 changed from tgccccCGCCcccac (SEQ ID NO:8) to tgccccTGACcccac (SEQ ID NO:9) using template pGL3b:Prm1 to generate pGL3Basic:Prm1^{GC}*⁽⁻⁸³⁴⁵⁾. Primers used: Kin423 (5′TGGAAGCTGCCCCTGACCCCACCCAGCTTC3′, SEQ ID NO:44) and the complementary oligonucleotide Kin424
2. GC at −8281 changed from gcccgGCCCccgccgga (SEQ ID NO:10) to gcccgGTTCccgccgga (SEQ ID NO:11) using template (a) pGL3b:Prm1 to generate pGL3Basic:Prm1^{GC}*⁽⁻⁸²⁸¹⁾ and (b) pGL3Basic:Prm1^{GC}*^{(-8345,-7831)} to generate pGL3Basic:Prm1^{GC}*^{(-8345,-8281,-7831)}. Primers used: Kin478 (5′CTCCCTGCCCGGTTCCCGCCGGAAACC3′, SEQ ID NO: 45) and the complementary oligonucleotide Kin479
3. GC at −8146 changed from cGGGGGGTgggGGGCGGGGGGCgggccaa (SEQ ID NO:12) to cGGGGGTCgtgGGGTGGATGGCgggccaa (SEQ ID NO:13) using template (a) pGL3b:Prm1 to generate pGL3Basic:Prm1^{GC}*⁽⁻⁸¹⁴⁶⁾ and (b) pGL3Basic:Prm1^{GC}*^{(-8345,-8281,-7831)} to generate pGL3Basic:Prm1^{GC}*^{(-8345,-8281,-8146,-7831)}.Primers used: Kin510 (5′GTGCTGGCGGGGGTCGTGGGGCGGGGGGCG3′, SEQ ID NO: 46) and the complementary oligonucleotide Kin511, as well as Kin512 (5′GGGGTCGTGGGGTGGATGGCGGGCCAAGAC3′, SEQ ID NO:47) and the complementary Kin513
4. GC at −7831 changed from tcactGCCCcctcatct (SEQ ID NO:14) to tcactGTCCtctcatct (SEQ ID NO:15) using template (a) pGL3b:Prm1 to generate pGL3Basic:Prm1^{GC}*⁽⁻⁷⁸³¹⁾, (b) pGL3b:Prm1B to generate pGL3Basic:Prm1B^{GC}*⁽⁻⁷⁸³¹⁾ and (c) pGL3Basic: Prm1^{GC}*⁽⁻⁸³⁴⁵⁾ to generate pGL3Basic: Prm1^{GC}*^{(-8345,-7831)}. Primers used: Kin361 (5′TCCGTCTCTCACTGTCCTCTCATCTGGAGCCC3′, SEQ ID NO:48) and the complementary oligonucleotide Kin362
5. GC at −6717 changed from tctgtcctCCCAcccca (SEQ ID NO:22) to tctgtcctATCAcccca (SEQ ID NO:23) using template pGL3Basic:Prm1D to generate pGL3Basic: Prm1D^{GC}*⁽⁻⁶⁷¹⁷⁾. Primers used: Kin502 (5′CATCCCTCTGTCCTATCACCCCACCCCTGG 3′, SEQ ID NO:49) and the complementary oligonucleotide Kin503
6. GC at −6206 changed from cagcggccCCCAcccgt (SEQ ID NO:28) to cagcggccTACAcccgt (SEQ ID NO:29) using template pGL3Basic:Prm1I to generate pGL3Basic:Prm1I^{GC}*⁽⁻⁶²⁰⁶⁾. Primers used: Kin506 (5′GCTGCCAGCGGCCTACACCCGTCCCAGC3′, SEQ ID NO:50) and the complementary oligonucleotide Kin507

To mutate a putative repressor site at −6800, herein referred to as WT1(X), the previously generated plasmid pGL3Basic: Prm1D^{GC}*^{(-6717,-6206)}, also referred to herein as pGL3Basic: Prm1D^{WT1(a)}*^{(b)}*, was used as template versus the primers Kin802 (5′ CCATGCGATGCCACCACCCCAGCCCTC 3′, SEQ ID NO:51) and the complementary oligonucleotide Kin803 to generate the plasmid pGL3Basic:Prm1D^{WT1(a)}*^{(b)}*^{(X)}*. The identity and fidelity of the resulting pGL3Basic: Prm1D^{WT1(a)}*^{(b)}*^{(X)}* plasmid was verified by DNA sequence analysis.

### Cell Culture

Human erythroleukemic (HEL) 92.1.7 cells, obtained from the American Type Culture Collection (ATCC), were cultured in RPMI 1640, 10% fetal bovine serum (FBS).Human erythroleukemia (HEL) 92.1.7 cells were obtained from the American Type Culture Collection (ATCC) and were grown in RPMI 1640, 10% fetal bovine serum (FBS). Human embryonic kidney (HEK) 293 cells were obtained from the ATCC and cultured in MEM, 10% FBS. EA.hy926 cells were obtained from were obtained from the Tissue Culture Facility of the University of North Carolina Lineberger Comprehensive Cancer Center (Chapel Hill, N.C.) and were cultured in Dulbecco's modified Eagle's medium (DMEM) and 10% FBS. WI-38 cells were obtained from the ATCC and cultured in MEM, 10% FBS, essential amino acids. Primary human aortic smooth muscle cells (1° hAoSMCs) were purchased from Cascade biologics and were cultured in M199, 10% FBS. All cells were grown at 37° C. in a humid environment with 5% CO₂.

### Assay of Luciferase Activity

HEL and EA.hy 926 cells were co-transfected with the various pGL3Basic-recombinant plasmids (2 mg), encoding firefly luciferase, along with pRL-TK (200 ng), encoding renilla luciferase, using DMRIE-C® transfection reagent. HEK293, WI-38, and 1° hAoSMCs were co-transfected with the pGL3Basic-recombinant plasmids (2 mg), along with pRL-TK (200 ng), using Effectene transfection reagent. In all cases, cells co-transfected with pRL-TK (200 ng) in the presence of either the pGL3Control vector (firefly luciferase gene under the control of the SV40 promoter; 2 mg) or the promoter-less pGL3Basic empty vector (2 mg), which served as positive and negative controls, respectively. Firefly and renilla luciferase expression was assayed some 48 h post-transfection using the Dual-Luciferase Reporter Assay System™, as described (Coyle et al., 2005). Relative firefly to renilla luciferase activity (arbitrary units) was calculated as a ratio and was expressed in relative luciferase units (RLU). To investigate the effect of over-expression of exon 5 (+ or −) or KTS (+ or −) isoforms of WT1 on Prm1-directed gene expression, HEL cells were co-transfected with either pGL3b:Prm1 or pGL3b:Prm1D (1.5 μg) plus 200 ng of pRL-TK along with either pcDNA3:WT1 (+/+), pcDNA3:WT1 (+/−), pcDNA3:WT1 (−/+), pcDNA3:WT1 (−/−) (0.5 μg), or as a control, pcDNA3 (0.5 μg). Cells were harvested 48 h post-transfection and assayed for luciferase activity, as above, or subjected to western blot or RT-PCR analysis. The plasmids pcDNA3:WT1 (+/+), pcDNA3:WT1 (+/−), pcDNA3:WT1 (−/+) and pcDNA3:WT1 (−/−) were generously donated by Dr. Charles T. Roberts JR, Oregon National Primate Research Center, Oregon, USA and have been previously described (Tajinda et al., 1999). For PMA studies, approximately 32 h post-transfection, the medium was supplemented with PMA (100 nM), with PMA (100 nM) and PD98059 (10 μM) or, as a control, with vehicle [0.1% (v/v) DMSO]. After 16 h, cells were assayed for firefly and renilla luciferase using the Dual-Luciferase Reporter Assay System™ as previously described (Coyle et al., 2005). The plasmid pCMV5-NAB1, containing the entire coding sequence for NAB1, was generously donated by Dr. Gerald Thiel, University of Saarland Medical Center, Homburg, Germany, and has been described previously (Thiel et al., 2000). To investigate the effect of over-expression of NAB1 on the PMA-mediated induction of Prm1-directed gene expression, HEL cells were transiently co-transfected with pGL3b:Prm1 (2 μg) plus pRL-TK (200 ng) along with either pCMV5 (control) or pCMV:NAB1 (1 μg). Cells were harvested 48 h post-transfection and assayed for luciferase activity, as above.

### Western Blot Analysis

The expression of WT1, Sp1, Egr1, NF-E2, GATA-1 and Ets-1 proteins in HEL cells was confirmed by western blot analysis. Briefly, whole cell protein was resolved by SDS-PAGE (10% acrylamide gels) and transferred to polyvinylidene difluoride (PVDF) membrane according to standard methodology. Membranes were screened using anti-WT1, anti-NF-E2, anti-Sp1, anti-Egr1, anti-GATA-1 or anti-Ets-1 sera in 5% non fat dried milk in 1×TBS (0.01 M Tris/HCl, 0.1 M NaCl) for 2 h at room temperature followed by washing and screening using goat anti-rabbit horseradish peroxidase (sc-2204) followed by chemiluminescence detection, as described by the supplier (Roche Applied Science). For PMA studies, HEL cells were pre-incubated for the indicated amount of time with 100 nM PMA, with 10 μM PD98059, with 100 nM PMA plus 10 μM PD98059 or, as a control, with vehicle [0.1% (v/v) DMSO]. Whole cell protein (60 μg per lane) was resolved by SDS-PAGE (10% acrylamide gels) and transferred to polyvinylidene difluoride (PVDF) membrane according to standard methodology. Membranes were screened using anti-WT1, anti-Sp1, anti-Egr-1 or anti-HDJ2 sera in 5% non fat dried milk in 1×TBS (0.01 M Tris/HCl, 0.1 M NaCl) for 2 h at room temperature followed by washing and screening using goat anti-rabbit horseradish peroxidase (sc-2204) followed by chemiluminescence detection, as described by the supplier (Roche Applied Science). HEL cells were co-transfected with either the empty vector pcDNA3.1(−), or with pcDNA3.1(−): hTF, pPrm1^{GC-8146}:hTF, pPrm1B:hTF, pPrm1D^{WT1(a)}*^{,WT1(b)}*:hTF (2 mg), encoding the full length cDNA for human tissue factor (hTF) under the control of the cytomegalovirus (CMV) promoter or Prm1 derivatives, respectively. All transfections were carried out using 2 mg of the respective plasmids and using DMRIE-C® transfection reagent. Cells were harvested 48 hr post-transfection and aliquots (60 mg/lane) were analysed by SDS-PAGE/western blot analysis. As a positive control for hTF expression, 60 mg/lane of a commercial control Tissue-Factor Biomass was also analysed. Briefly, protein was resolved by SDS-PAGE (10% acrylamide gels) and transferred to polyvinylidene difluoride (PVDF) membrane according to standard methodology. Membranes were screened using anti-hTF sera in 5% non fat dried milk in 1×TBS (0.01 M Tris/HCl, 0.1 M NaCl) for 2 h at room temperature. Thereafter, membranes were washed and screened using goat anti-rabbit horseradish peroxidase (sc-2204), followed by chemiluminescence detection as previously described.

### Electrophoretic Mobility Shift and Supershift Assays

Nuclear extract was prepared from HEL cells as previously described (Coyle et al., 2005). Oligonucleotides corresponding to the sense (5′ end-labelled with biotin) and antisense strands of each probe (90 μM) were annealed by heating at 95° C. for 2 min followed by slow cooling to room temperature. The identities and sequences of the biotin-labelled oligonucleotide probes and the non-labelled competitor/non-competitor oligonucleotides are listed in the expanded Materials and Methods section below. Initially, serial dilutions of each probe were incubated with nuclear extract (2.5 μg total protein) for 20 min at room temperature in 1× Binding Buffer [20% glycerol, 5 mm MgCl₂, 2.5 mm EDTA pH 8.0, 250 mm NaCl, 50 mm Tris-HCl pH 8.0 and 0.25 mg ml⁻¹ poly (dI-dC; Sigma)]. Protein-DNA complexes were subjected to electrophoresis through 6% DNA retardation gels (Invitrogen) in Tris borate, EDTA (TBE) buffer for 1-2 h at room temperature and then transferred to Biodyne® B positively-charged nylon membrane (PaII). Thereafter, detection was carried out using the Chemiluminescence Nucleic Acid Detection Module, as described by the manufacturer. Once the optimal concentration of each probe was determined, binding reactions were set up by incubating nuclear extract (2.5 μg total protein) with/without 300-fold molar excesses of non-labelled double-stranded competitors/non-competitors in 1× Binding Buffer for 20 min at room temperature. The appropriate concentration of biotin-labelled probe was then added and mixtures were incubated for 20 min at room temperature after which electrophoresis, transfer and detection were carried out, as before. For supershift assays, nuclear extract (2.5 μg total protein) was pre-incubated with 3 μg of anti-NF-E2, anti-Sp1, anti-Egr1, anti-WT-1 or anti-cJun sera for 2 h at 4° C. Thereafter, the nuclear extract-antibody mixtures were incubated for 20 min at room temperature with the appropriate biotin-labelled double-stranded probe, as described in the expanded Materials and Methods section below. The sequences of the probes used were as follows:

| | |
|---|---|
| 1. | GC^{-8345,-8281} probe |
| | (Kin733; 5′[Btn]GAAGCTGCCCCCGCCCCCACCCA |
| | GCTTCCTGACTTTGGCTGTGTCCAGAGCTAAGAATAGACGCTCCC |
| | TGCCCGGCCCCCGCCGGAAACCG3′, nu −8350 to −8260 |
| | of Prm1, SEQ ID NO: 52) |
| 2. | GC⁻⁸¹⁴⁶ probe |
| | (Kin737; 5′[Btn]GTGCTGGCGGGGGGTGGGGGGCGG |
| | GGGGCGGGCCAAGACCGG3′, nu −8153 to −8118 of |
| | Prm1, SEQ ID NO: 53) |
| 3. | GC⁻⁷⁸³¹ probe |
| | (Kin739; 5′[Btn]TCCTCCGTCTCTCACTGCCCCCTCATC |
| | TGGAGCCCCAG3′, nu −7842 to −7805 of Prm1, |
| | SEQ ID NO: 54) |
| 4. | GC⁻⁶⁷¹⁷ probe |
| | (Kin762; 5′[Btn]CACCCCCCATCCCTCTGTCCTCCCA |
| | CCCCACCCCTGGAAG3′, nu −6730 to −6691 |
| | of Prm1, SEQ ID NO: 55) |
| 5. | GC⁻⁶²⁰⁶ probe |
| | (Kin764; 5′[Btn]GCCGCGGGCTGCCAGCGGCCCCCACC |
| | CGTCCCAGCTCGGC3′, nu −6218 to −6178 of Prm1, |
| | SEQ ID NO: 56) |

Only forward biotin-labelled oligonucleotides are listed above. Sequences of the corresponding non-labelled complementary oligonucleotides are omitted. The sequences of the competitor/non-competitor oligonucleotides used were as follows:

| | |
|---|---|
| 1. | Prm1⁻⁸³⁴⁵ competitor |
| | (Kin458; 5′CTGGAAGCTGCCCCCGCCCCCACCC AG3′, |
| | nu −8453 to −8327 of Prm1, SEQ ID NO: 57) |
| 2. | Prm1⁻⁸²⁸¹ competitor |
| | (Kin742; 5′CTCCCTGCCCGGCCCCCGCCGGAAACCGC3′, |
| | nu −8287 to −8259 of Prm1, SEQ ID NO: 58) |
| 3. | Prm1⁻⁸¹⁴⁶ competitor |
| | (Kin745; 5′GTGCTGGCGGGGGGTGGGGGGCGGGGGGCGGGCCAAGACCGG3', |
| | nu −8153 to −8112 of Prm1, SEQ ID NO: 59) |
| 4. | Prm1⁻⁷⁸³¹ competitor |
| | (Kin798; 5′TCCTCCGTCTCTCACTGCCCCCTCATCTGGAGCCCCAG3′, |
| | nu −7842 to −7805 of Prm1, SEQ ID NO: 60) |
| 5. | Prm1⁻⁶⁷¹⁷ competitor |
| | (Kin779; 5′CACCCCCCATCCCTCTGTCCTCCCACCCCACC CCTGGAAG3′, |
| | nu −6730 to −6691 of Prm1, SEQ ID NO: 61) |
| 6. | Prm1⁻⁶²⁰⁶ competitor |
| | (Kin780; 5′GCCGCGGGCTGCCAGCGGCCCCCACCCGTCCCAGCTCGGC3′, |
| | nu −6218 to −6179 of Prm1, SEQ ID NO: 62) |
| 7. | WTE consensus |
| | (Kin748; 5′CGAGTGCGTGGGAGTAGAATT3′, SEQ ID NO: 63) |
| 8. | Sp1 consensus |
| | (Kin651; 5′ATTCGATCGGGGCGGGGCGAGC3′, SEQ ID NO: 64) |
| 9. | Egr1 consensus |
| | (Kin746; 5′GGATCCAGCGGGGGCGAGCGGGGGCGA 3′, SEQ ID NO: 65) |
| 10. | non-specific A |
| | (Kin484; 5′GGGCCGAGGACAGGTGAAGTGGGGACAG 3′, SEQ ID NO: 66) |
| 11. | non-specific B |
| | (Kin450; 5′GCCAGACTGACTCAGTTTCCC3′, SEQ ID NO: 67) |

### Chromatin Immunoprecipitation (ChIP) Assays

Chromatin immunoprecipitation (ChIP) assays were performed essentially as described (Koch et al., 2007). Specifically, HEL cells (1×10⁸) were pelleted, washed in ice-cold PBS and resuspended in serum-free RPMI 1640. Formaldehyde-cross linked chromatin was sonicated, as described (Koch et al., 2007), to generate fragments 500 bp to 1000 bp in length. Prior to immunoprecipitation (IP), chromatin was incubated with 60 μg normal rabbit IgG overnight at 4° C. on a rotisserie, after which 250 μl of salmon sperm DNA/protein A agarose beads (Millipore) were added and chromatin was precleared for 3 h at 4° C. with rotation. Thereafter, anti-WT1, anti-NF-E2, anti-Sp1, anti-Egr1, anti-GATA-1, anti-Ets-1 (10 μaliquots), normal rabbit IgG (10 μg), or a “no antibody” control were used for immunoprecipitation. Following elution, cross-links were reversed by incubation at 65° C. overnight followed by protease digestion with proteinase K (Sigma; 9 μl of 10 mg/ml) at 45° C. for 7 h. After precipitation, samples were resuspended in 50 μl dH₂O. PCR analysis was carried out using 2-3 μl of ChIP sample as template or, as a positive control, with an equivalent volume of a 1:20 dilution of the input chromatin DNA. The identities of the primers used for the ChIP PCR reactions, as well as their sequences and corresponding nucleotides within Prm1 are listed below.

| | |
|---|---|
| 1. | Kin462 (5′CGAGACCCTGCAGGCAGACTGGAG3′; |
| | −8460 to −8437, SEQ ID NO: 68) |
| 2. | Kin463 (5′GAGATGGGGAAACTGAGGCACAAAG3′; |
| | −8030 to −8006, SEQ ID NO: 69) |
| 3. | Kin468 (5′GCCTTGCAGAGATGTGGTGAGGC3′; |
| | −7978 to −7973, SEQ ID NO: 70) |
| 4. | Kin467 (5′GAGGTGAGCTAGGAAGACATCTTG3′; |
| | −7630 to −7607, SEQ ID NO: 71) |
| 5. | Kin233 (5′GAGAGGTACCGCTCCAAAGCCACCTCC G3′; |
| | −6848 to −6831, SEQ ID NO: 72) |
| 6. | Kin144 (5′AGAGACGCGTCGCTTCCTCGGGAGCCTCA3′; |
| | −6455 to −6437, SEQ ID NO: 73) |
| 7. | Kin456 (5′CTTCCCCAGAAGGCTGTAGGGTGTC3′; |
| | −6368 to −6344, SEQ ID NO: 74) |
| 8. | Kin109 (5′AGAGACGCGTCTTCAGAGACCTCATCTGCGGGG3′; |
| | −5917 to −5895, SEQ ID NO: 39) |

For PMA studies, HEL cells were pre-incubated with vehicle [0.1% (v/v) DMSO; 16 h] or with 100 nM PMA for 5 h, 8 h or 16 h. PMA-stimulated HEL cells (1×10⁸) were scraped to remove from culture flasks, and then both PMA-stimulated and vehicle-treated cells were pelleted, washed in ice-cold PBS and resuspended in serum-free RPMI 1640. Formaldehyde-cross linked chromatin was sonicated to generate fragments 500 bp to 1000 bp in length. Chromatin samples were immunoprecipitated with 10 μg of anti-WT1, anti-Sp1 or anti-Egr1. The primers used for the ChIP PCR reactions, their sequences and corresponding nucleotides within Prm1 are listed below.

| | |
|---|---|
| 9. | Kin462 (5′CGAGACCCTGCAGGCAGACTGGAG3′; |
| | −8460 to −8437, SEQ ID NO: 68) |
| 10. | Kin463 (5′GAGATGGGGAAACTGAGGCACAAAG3′; |
| | −8030 to −8006, SEQ ID NO: 69) |
| 11. | Kin456 (5′CTTCCCCAGAAGGCTGTAGGGTGTC3′; |
| | −6368 to −6344, SEQ ID NO: 74) |
| 12. | Kin109 (5′AGAGACGCGTCTTCAGAGACCTCATC |
| | TGCGGGG3′; −5917 to −5895, |
| | SEQ ID NO: 39) |

Reverse transcriptase-polymerase chain reaction (RT-PCR)

Total RNA was isolated from HEL 92.1.7 cells (5×10⁶ approximately) using TRIzol reagent (Invitrogen Life Technologies). RT-PCR was carried out with DNase 1-treated total RNA using oligonucleotide primers to specifically amplify TPα and glyceraldehyde-3-phosphate dehydrogenase (GAPDH) mRNA sequences, as previously described (Miggin et al., 1998). The following primers were used:

1. Kin16:
(SEQ ID NO: 75)
TPα forward 5′-GAGATGATGGCTCAGCTCCT-3′
2. DT75:
(SEQ ID NO: 76)
TPα reverse 5′-CCAGCCCCTGAATCCTCA-3′
3. Kin291:
(SEQ ID NO: 77)
GAPDH forward 5′-CCACAGTCCATGCCATCAC-3′
4. DT91:
(SEQ ID NO: 78)
GAPDH forward 5′-TGAAGGTCGGAGTCAACG-3′
5. DT92:
(SEQ ID NO: 79)
GAPDH reverse 5′-CATGTGGGCCATGAGGTC-3′

Following agarose gel electrophoresis, PCR products were transferred to Biodyne® B positively-charged nylon membrane (PaII) using standard methodology (Sambrook et al., 1989). Southern blot analysis of the RT-PCR products was carried out using oligonucleotide probes (5′ end-labelled with biotin) specific for TPα (Kin 583: 5′[Btn]CTGTCCCGCACCACGGA GAAG3′, SEQ ID NO:80) and GAPDH (Kin 584: 5′[Btn]CACCCAGAAGACTGTGGATGGC3′, SEQ ID NO:81] mRNA sequences and detection was carried out using the Detector™ HRP Chemiluminescent Blotting Kit (KPL), as described by the manufacturer.

### Confocal Microscopy

HEL 92.1.7 cells were seeded at ˜1×10⁵ cells/ml in 2 ml normal growth media (RPMI 1640, 10% FBS) in 6-well plates containing Poly-L-lysine coated coverslips. Cells were grown at 37° C. for 24 h prior to incubating cells with 100 nM PMA for 0, 1, 5, 8, 16 and 24 h. Thereafter, cells were fixed in 3.4% paraformaldehyde and permeabilized with 0.2% Triton X-100 for 10 min on ice, after which cells were immunolabelled with anti-WT1 (1:1000 of 2 μg/μl stock) and AlexaFluor488 conjugated anti-rabbit, followed by counterstaining with DAPI (1 μg/ml in H₂O). Images were obtained using Carl Zeiss Lazer Scanning System LSM510 and Zeiss LSM Imaging software for acquiring multi-channel images with filters appropriate for enhanced DAPI & AlexaFluor488.

### Statistical Analysis

Statistical analysis of differences were routinely analysed using the two-tailed Student's unpaired t-test and two-way ANOVA. All values are expressed as mean±standard error of the mean (SEM). P-values<0.05 were considered to indicate statistically significant differences and *, **, ***, **** indicate p<0.05, p<0.01, p<0.001 and p<0.0001, respectively.

### Supplemental Materials and Methods

### Construction of Luciferase-Based Genetic Reporter Plasmids

To identify elements required for Prm1 activity, a series of 5′- and 3′-deletion subfragments were subcloned into the Kpn1-Mlu1 site of pGL3Basic. Specifically, for all 5′ deletions, PCR fragments were generated using the antisense primer Kin109 (5′AGAG ACGCGTCTTCAGAGACCTCATCTGCGGGG3′; complementary to nucleotides −5917 to −5895 of Prm1, SEQ ID NO:39, where the underlined sequence corresponds to a Mlu1 cloning site), in combination with specific sense primers designed to amplify progressively shorter fragments. The list below identifies the recombinant plasmids encoding 5′ deletion fragments of Prm1 generated in pGL3Basic (pGL3b), as well as the identity, sequence and corresponding nucleotides (nu) of the specific sense primer used for each fragment. In each case, the − designation indicates nucleotides 5′ of the translational ATG start codon (designated +1) and underlined sequences represent the Kpn1 cloning site.

| | |
|---|---|
| 1. | pGL3b:Prm1B; Primer Kin191 |
| | (5′GAGAGGTACCGTGAGGCTTAAGCTAAATGC3′; |
| | nu −7962 to −7943, SEQ ID NO: 82) |
| 2. | pGL3b:Prm1BΔGata/Ets; Primer Kin363 |
| | (5′GAGAGGTACCTGGGGACAGGACAGCCTTCCTCCG3′; |
| | nu −7859 to −7836, SEQ ID NO: 83) |
| 3. | pGL3b:Prm1B□; Primer Kin192 |
| | (5′GAGAGGTACCCATGCAATTCCTGTCACTGCC3′; |
| | nu −7717 to −7697, SEQ ID NO: 84) |
| 4. | pGL3b:Prm1C; Primer Kin203 |
| | (5′GAGAGGTACCGGCAGGCCTGGTTTCAGGTCTC3′; |
| | nu −7504 to −7483, SEQ ID NO: 85) |
| 5. | pGL3b:Prm1D; Primer Kin233 |
| | (5′GAGAGGTACCGCTCCAAAGCCACCTCCG3′; |
| | nu −6848 to −6831, SEQ ID NO: 72) |
| 6. | pGL3b:Prm1E; Primer Kin234 |
| | (5′GAGAGGTACCCTACTGTGTGCCGCGTTC3′; |
| | nu −6648 to −6631, SEQ ID NO: 86) |
| 7. | pGL3b:Prm1HΔ; Primer Kin390 |
| | (5′GAGAGGTACCGACTCCAAGTCAGCCAGGCCC3′; |
| | nu −6320 to −6300, SEQ ID NO: 87) |
| 8. | pGL3b:Prm1K; Primer Kin392 |
| | (5′GAGAGGTACCTCAGTTTCCCTGGGAGGTCCC3′; |
| | nu −6067 to −6047, SEQ ID NO: 88) |
| 9. | pGL3b:Prm1L; Primer Kin393 |
| | (5′GAGAGGTACCCAGCCCTCGCCCCACCCTC3′; |
| | nu −6010 to −5992, SEQ ID NO: 89) |

For all 3′ deletions, PCR fragments were generated using the antisense primer Kin144 (5′AGAG ACGCGTCGCTTCCTCGGGAGCCTCA3′, complementary to nucleotides −6455 to −6437, SEQ ID NO:73, where the underlined nucleotides correspond to an Mlu1 cloning site). The following lists the recombinant pGL3b plasmids encoding 3′ deletion fragments of Prm1, as well as the identity of the specific sense primer used for each fragment.
1. pGL3b:Prm1B□ 0 3′ deletion; Primer Kin192 (SEQ ID NO:84)
2. pGL3b:Prm1C 3′ deletion; Primer Kin203 (SEQ ID NO:85)
3. pGL3b:Prm1E 3′ deletion; Primer Kin234 (SEQ ID NO:86)
   A 245 bp-deletion fragment encoding the Prm1 sequence between −7962 and −7718 was amplified by PCR using pGL3b:Prm1 as template and the primers Kin191 (SEQ ID NO:82) and Kin565 (5′AGAG ACGCGTTATAAAGCTTTGGAGGCAAGAGAG 3′; nu −7741 to −7718, SEQ ID NO:90). This fragment was then subcloned into vector pGL3control, upstream of the SV40 promoter, to generate the plasmid pGL3control:Prm1GATA/Ets. The identity and fidelity of all recombinant plasmids was verified by DNA sequence analysis.

### Site-Directed Mutagenesis

The identities of the Prm1 elements subjected to site-directed mutagenesis, with their starting positions in brackets, the nucleotides that were changed, templates used and names of the corresponding plasmids generated, as well as the identity, sequence and corresponding nucleotides of the specific primers used are listed below.

1. GATA (−7890), from cttgtTATCtcag (SEQ ID NO:16) to cttggTAGCtcag (SEQ ID NO:17) using template pGL3b:Prm1B to generate pGL3b:Prm1B^{GATA(-7890)}*. Primers Kin482 (5′GTCATTGTCCTTGGTAGCTCAGGGCCGAGGAC3′, SEQ ID NO:91) and complementary Kin483
2. Ets (−7870), from gacagAGGAagtgggga (SEQ ID NO:18) to gacagGTGAagtgggga (SEQ ID NO:19) using template pGL3b:Prm1B to generate pGL3b:Prm1B^{Ets1(-7870)}. Primers Kin484 (5′GGGCCGAGGACAGGTGAAGTGGGGACAG3′, SEQ ID NO:66) and complementary Kin485
3. Ets (−7870), from gacagAGGAagtgggga (SEQ ID NO:18) to gacagGTGAagtgggga (SEQ ID NO:19) using template pGL3b:Prm1B^{GATA(-7890)}* to generate pGL3B:Prm1B^{GATA(-7890)}*^{,Ets(-7870)}*. Primers Kin484 (5′GGGCCGAGGACAGGTGAAGTGGGG ACAG3′, SEQ ID NO:66) and complementary Kin485
4. Ets (−7805), from gccccacaTCCTcctcc (SEQ ID NO:20) to gccccacaTCACcctcc (SEQ ID NO:21) using template pGL3b:Prm1BΔGata/Ets to generate pGL3b:Prm1B□Gata/Ets^{Ets(-7805)}*. Primers Kin486 (5′CCCAGCCCCACATCACCCTCCCCCAAC3′, SEQ ID NO:92) and complementary Kin487
5. NF-E2/AP1 (−6080), from ccagaCTGActcagtttccct (SEQ ID NO:32) to ccagaCACActcagtttcc ct (SEQ ID NO:33) using template pGL3b:Prm1HΔ to generate pGL3b:Prm1H□^{NF-E2/AP1(-6080)}*. Primers Kin431 (5′CTGGTCACAGCCAGACACACTCAGTTTCCCTGG3′, SEQ ID NO:93) and complementary Kin432
6. Sp1/Egr1 (−6294), from cgaggGGCGtggcca (SEQ ID NO:24) to cgaggAACAtggcca (SEQ ID NO:25) using template pGL3b:Prm1HΔ to generate pGL3b:Prm1H□^{Sp1/Egr1(-6294)}*. Primers Kin425 (5′CCCGGGCCCGAGGAACATGGCCAGCGCAGG3′, SEQ ID NO:94) and complementary Kin426
7. Sp1/Egr1 (−6278), from cgcagggtGGGCggggctg (SEQ ID NO:26) to cgcagggtGTATGgggctg (SEQ ID NO:27) using template pGL3b:Prm1HΔ to generate pGL3b:Prm1H□^{Sp1/Egr1 (-6278)}. Primers Kin427−(5′GCCAGCGCAGGGTGTATGGGGCTGATGAGAGAC3′, SEQ ID NO:95) and complementary Kin428
8. Sp1/Egr1 (−6098), from tgggcccGCCCctgg (SEQ ID NO:30) to tgggcccAATCctgg (SEQ ID NO:31) using template pGL3b:Prm1HΔ to generate pGL3b:Prm1H□^{Sp1/Egr1(-6098)}*. Primers Kin429 (5′CCTCTGCTGGGCCCAATCCTGGTCACAGCCAG3′, SEQ ID NO:96) and complementary Kin430
9. Sp1/Egr1 (−6022), from tctgcccGCCCccagccct (SEQ ID NO:32) to tctgcccTAACccagccct (SEQ ID NO:33) using template pGL3b:Prm1HΔ to generate pGL3b: Prm1H□^{Sp1/Egr1(-6022)}*. Primers Kin433 (5′CCTCCCTCTGCC CTAACCCAGCCCTCGCCC3′, SEQ ID NO:97) and complementary Kin434
10. Sp1/Egr1 (−6007), from ccctcGCCCCACCctcgg (SEQ ID NO:36) to ccctcGCAATACCctcgg cgagGGTATTGCgagggcga (SEQ ID NO:37) using template pGL3b:Prm1HΔ to generate pGL3b: Prm1H□^{Sp1/Egr1(-6007)}*. Primers Kin435 (5′CCCAGCCCTCGCAATACCCTCGGCGCCCGC3′, SEQ ID NO:98) and complementary Kin436
11. Sp1/Egr1 (−6022), from tctgcccGCCCccagccct (SEQ ID NO:34) to tctgcccTAACccagccct (SEQ ID NO:35) using template pGL3b:Prm1H□^{Sp1/Egr1(-6294)}* to generate pGL3b: Prm1H□AP1^{Sp1/Egr1(-6294,-6022)}*.Primers Kin433 (5′CCTCCCTCTGCCCTAACCCAGCCCTCGCCC3′, SEQ ID NO:97) and complementary Kin434
12. Sp1/Egr1 (−6007), from ccctcGCCCCACCctcgg (SEQ ID NO:36) to ccctcGCAATACCctcgg (SEQ ID NO:37) using template pGL3b: Prm1H□^{Sp1/Egr1(-6022)}* to generate pGL3b: Prm1H□^{Sp1/Egr1(-6022,-6007)}*. Primers Kin435 (5′CCCAGCCCTCGCAATACCCTCGGCGCCCGC3′, SEQ ID NO:98) and complementary Kin436
13. Sp1/Egr1 (−6007), from ccctcGCCCCACCctcgg (SEQ ID NO:36) to ccctcGCAATACCctcgg (SEQ ID NO:37) using template pGL3b:Prm1L to generate pGL3b:Prm1L^{Sp1/Egr1(-6007)}*. Primers Kin435 (5′CCCAGCCCTCGCAATACCCTCGGCGCCCGC3′, SEQ ID NO:98) and complementary Kin436

### Electrophoretic Mobility Shift and Supershift Assays

The identities and sequences of the forward biotin-labelled oligonucleotide probes are listed below. Sequences of the corresponding non-labelled complementary oligonucleotides are omitted.

1. NF-E2/AP1 Probe; Kin750
(5′[Btn]CTGGTCACAGCCAGACTGACTCAGTTTCC
CTGGGAGGTC3′;nu −6087 to −6049,
SEQ ID NO: 99)
2. Sp1/Egr1⁻⁶²⁹⁴ Probe; Kin906
(5′[Btn]GGGCCCGAGGGGCGTGGCCAGCGC3′;
nu −6299 to −6276 of Prm1, SEQ ID NO: 100)
3. Sp1/Egr1⁻⁶²⁷⁸ Probe; Kin909
(5′[Btn]GCCAGCGCAGGGTGGGCGGGGCTGATGAG3′;
nu −6283 to −6255, SEQ ID NO: 101)
4. Sp1/Egr1^{-6022,-6007} Probe; Kin647
(5′[Btn]CTCCCTCTGCCCGCCCCCAGCCCTCGCCC
CACCCTCGGCGCCC3′;
nu −6027 to −5985, SEQ ID NO: 102)
5. GATA, Ets Probe; Kin699
(5′[Btn]CATTGTCCTTGTTATCTCAGGGCCGAGGACAGAGGA
AGTGGGGACAGGAC3′; nu −7898 to −7848,
SEQ ID NO: 103)

The identities and sequences of the forward non-labelled competitor/non-competitor oligonucleotides are listed below.

| | |
|---|---|
| 1. | NF-E2/AP1 Prm1; Kin830 |
| | (5′CTGGTCACAGCCAGACTGACTCAGTTTCCCTGGGAGGTC3′; |
| | nu −6087 to −6049, SEQ ID NO: 104) |
| 2. | NF-E2 consensus; Kin766 (5′TGGGGAACCTGTGCTGAGT |
| | CACTGGAG 3′, SEQ ID NO: 105) |
| 3. | cJun consensus; Kin649 |
| | (5′CGCTTGATGACTCAGCCGGAA3′, SEQ ID NO: 106) |
| 4. | Sp1/Egr1⁻⁶²⁹⁴; Kin908 |
| | (5′GGGCCCGAGGGGCGTGGCCAGCGC3′, |
| | nu −6299 to −6276 of Prm1, SEQ ID NO: 107) |
| 5. | Sp1/Egr1⁻⁶²⁷⁸; Kin911 |
| | (5′GCCAGCGCAGGGTGGGCGGGGCTGATGAG3′, |
| | nu −6283 to −6255 of Prm1, SEQ ID NO: 108) |
| 6. | Sp1/Egr1⁻⁶⁰²²; Kin452 |
| | (5′CTCCCTCTGCCCGCCCCCAGCCCTCG3′, |
| | nu −6027 to −6002 of Prm1, SEQ ID NO: 109) |
| 7. | Sp1/Egr1⁻⁶⁰⁰⁷; Kin454 |
| | (5′CCCCAGCCCTCGCCCCACCCTCGGCGCCCG3′, |
| | nu −6013 to −5984 of Prm1, SEQ ID NO: 110) |
| 8. | Sp1 consensus; Kin651 |
| | (5′ATTCGATCGGGGCGGGGCGAGC3′, SEQ ID NO: 64) |
| 9. | Egr1 consensus; Kin746 |
| | (5′GGATCCAGCGGGGGCGAGCGGGGGCGA3′, |
| | SEQ ID NO: 65) |
| 10. | WT-1 sequence; Kin748 |
| | (5′CGAGTGCGTGGGAGTAGAATT3′, SEQ ID NO: 63) |
| 11. | Prm1^{GATA}; Kin701 |
| | (5′CATTGTCCTTGTTATCTCAGGGCCGAG3′, |
| | nu −7897 to −7871 of Prm1, SEQ ID NO: 111) |
| 12. | Prm1^{Ets}; Kin703 |
| | (5′GGCCGAGGACAGAGGAAGTGGGGACAGGAC3′ |
| | nu −7877 to −7848 of Prm1, SEQ ID NO: 112) |
| 13. | GATA-1 consensus; Kin705 |
| | (5′TCCCTGATAAGACCCAGG3′, SEQ ID NO: 113) |
| 14. | Ets-1 consensus; Kin707 |
| | (5′GATCTCGAGCAGGAAGTTCGA3′, SEQ ID NO: 114) |
| 15. | non-specific; Kin335 |
| | (5′TGCGCCCGGCCTTCCATGCTCTTTGAC 3′, |
| | SEQ ID NO: 115) |

### Chromatin Immunoprecipitation (ChIP) Assays

The identities of the primers used for the ChIP PCR reactions, as well as their sequences and corresponding nucleotides within Prm1 are listed below.

| | |
|---|---|
| 13. | Kin456 (5′CTTCCCCAGAAGGCTGTAGGGTGTC3′; |
| | nu −6368 to −6344, SEQ ID NO: 74) |
| 14. | Kin109 (5′AGAGACGCGTCTTCAGAGACCTCATCTGCGGGG3′; |
| | nu −5917 to −5895, SEQ ID NO: 39) |
| 15. | Kin468 (5′GCCTTGCAGAGATGTGGTGAGGC3′; |
| | −7978 to −7973, SEQ ID NO: 70) |
| 16. | Kin467 (5′GAGGTGAGCTAGGAAGACATCTTG3′; |
| | −7630 to −7607, SEQ ID NO: 71) |
| 17. | Kin462 (5′CGAGACCCTGCAGGCAGACTGGAG3′; |
| | −8460 to −8437, SEQ ID NO: 68) |
| 18. | Kin463 (5′GAGATGGGGAAACTGAGGCACAAAG3′; |
| | −8030 to −8006, SEQ ID NO: 69). |

### EXAMPLES

### Example 1

### Functional Analysis of Promoter 1 of the Human TXA₂ Receptor Gene

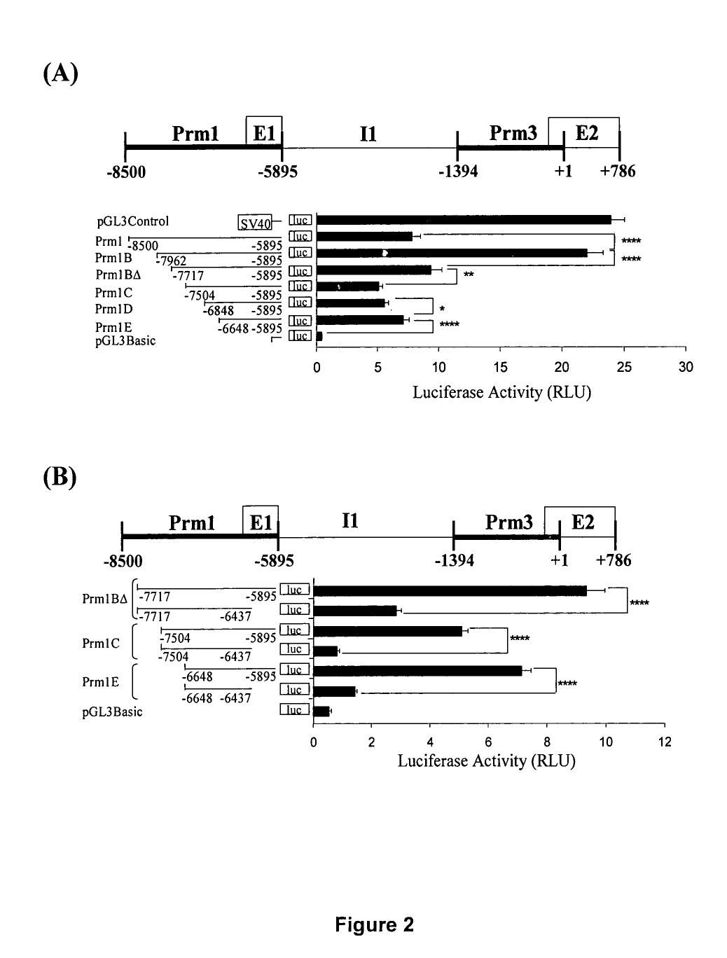

The aim of this investigation was to characterize promoter (Prm)1 of the human thromboxane (TX) A₂ receptor (TP) gene within the megakaryocytic human erythroleukemia (HEL) 92.1.7 cell line, seeking to identify the key factors regulating TP□ expression in platelets and related cell types. Prm1 is defined as nucleotides −8500 to −5895 upstream of the translational initiation codon (Coyle et al., 2002). A series of 5′ deletions was generated, where the 5′ nucleotide of each sub-fragment is indicated in brackets throughout. Through genetic reporter assays, the recombinant plasmid pGL3b:Prm1 directed 7.83±0.70 RLU in HEL cells (FIG. 2A), compared to 23.9±1.1 RLU directed by an SV40 promoter in the pGL3control vector, which acted as a reference. Deletion of Prm1 (−8500) to Prm1B (−7962) yielded a 2.8-fold increase in luciferase activity (p<0.0001). Further 5′ deletion to generate Prm1BΔ (−7717) resulted in a 2.4-fold decrease in luciferase expression (p<0.0001). Moreover, progressive 5′ deletion to generate Prm1C (−7504) yielded a further 1.8-fold reduction (p=0.0014), whilst deletion of nucleotides from Prm1D (−6848) to generate Prm1E (−6648) resulted in a 1.3-fold increase (p=0.0242) in luciferase expression. Hence, 5′ deletion analysis revealed two upstream repressor sequences (URS; between −8500 to −7962; −6848 to −6648) and two upstream activator sequences (UAS; between −7962 to −7717; −7717 to −7504) within Prm1. The Prm1E (−6648) sub-fragment directed luciferase expression comparable to that of the full-length Prm1, indicating that Prm1E contains core elements required to direct minimal Prm1 activity. Consistent with this, 3′ deletion of nucleotides −6437 to −5895 from Prm1BΔ, Prm1C and Prm1E significantly reduced luciferase expression (p<0.0001 in each case; FIG. 2B), to levels that were not substantially greater than that of pGL3Basic, such as in the case of Prm1E 3′ deletion. These data further suggest that the proximal Prm1E (−6648) contains the “core” elements required to direct minimal Prm1 activity.

### Example 2

### Identification of Functional NF-E2 and Overlapping Sp1/Egr1 Elements in Prm1

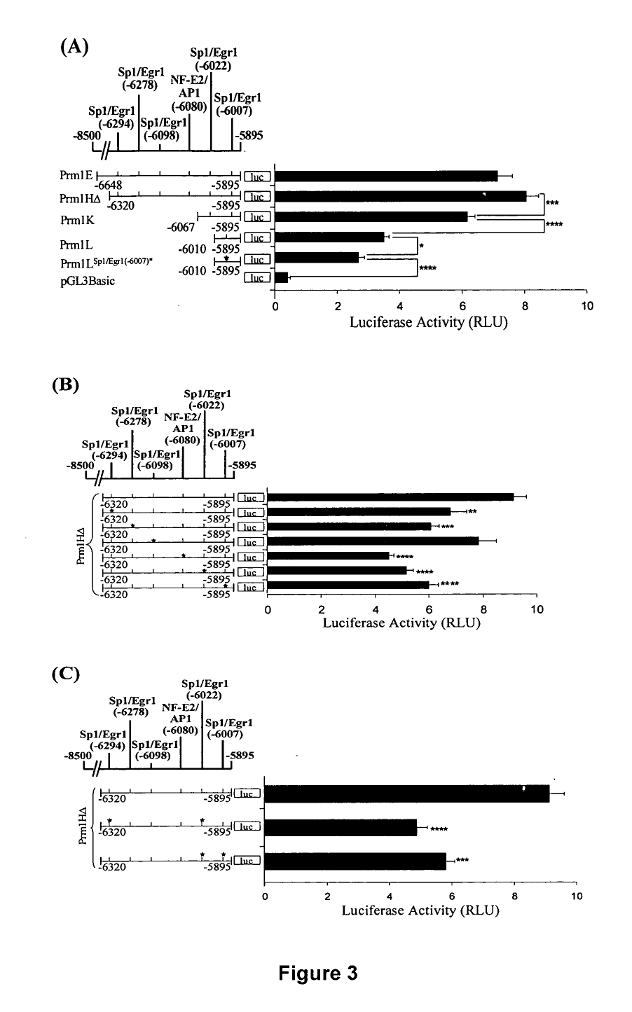

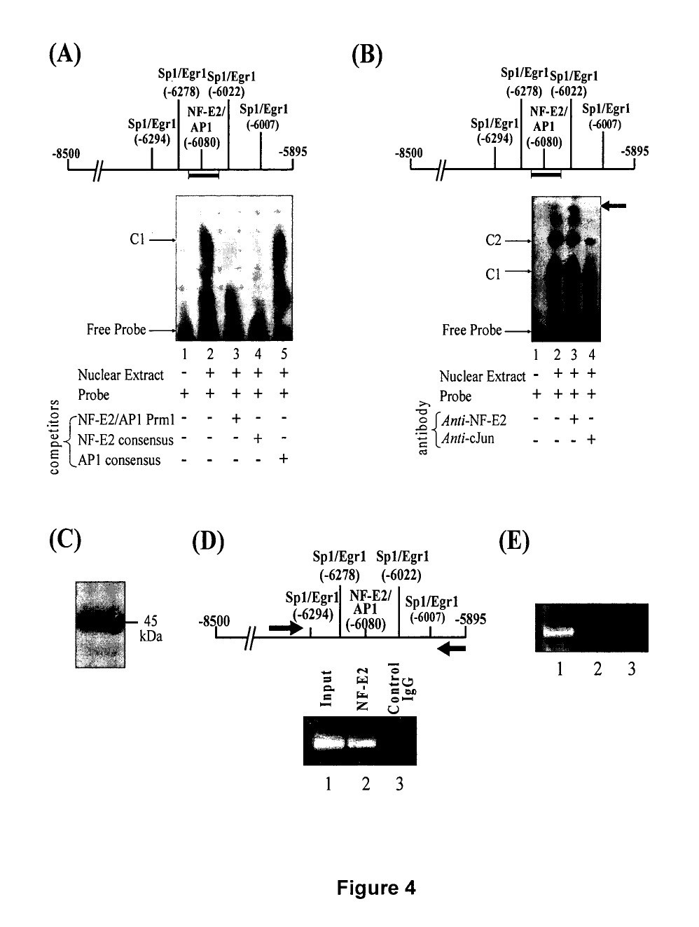

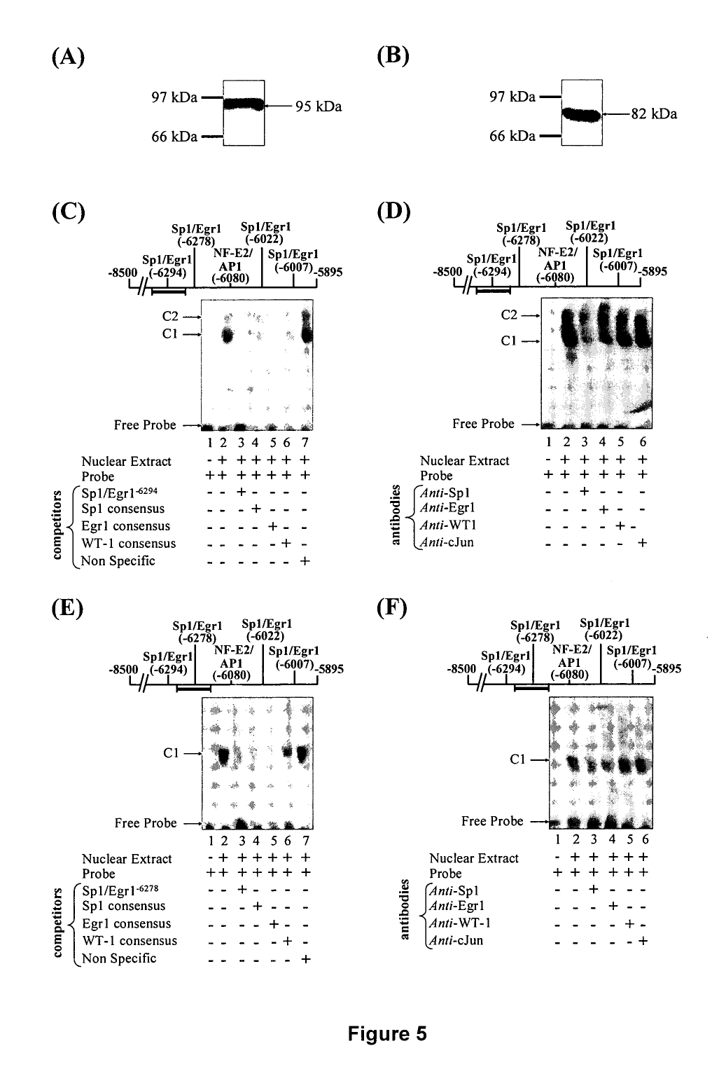

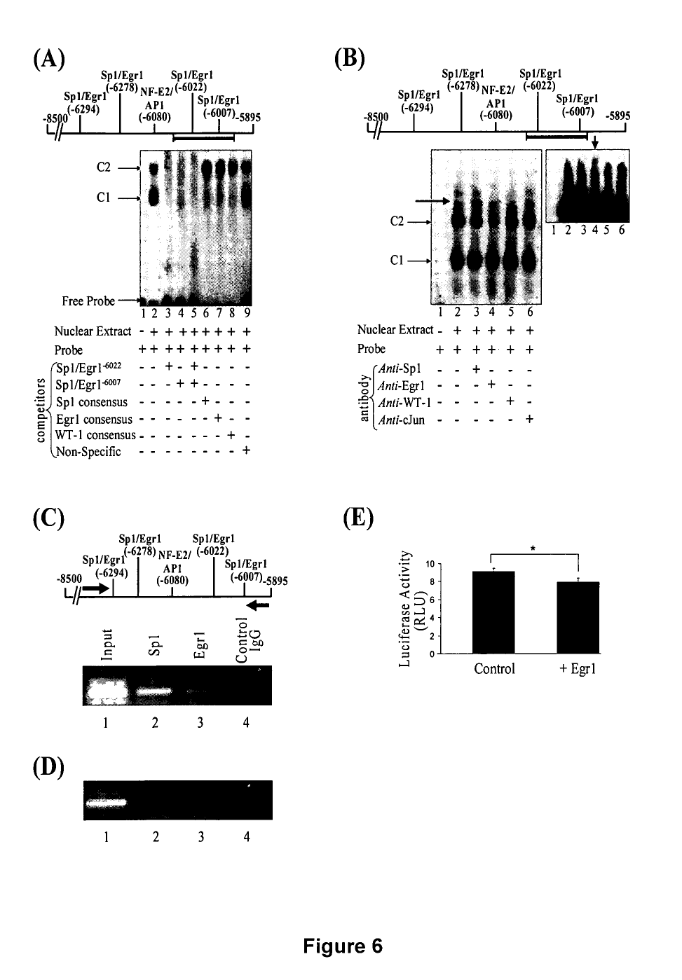

Successive 5′ deletions of Prm1E (−6648) further localized the positive regulatory element(s) between −6648 and −5895 (FIG. 3A). Deletion of nucleotides from −6648 to generate Prm1HD (−6320) did not affect luciferase expression, but generation of Prm1K (−6067) and Prm1L (−6010) led to 1.3-fold (p=0.0003) and 1.8-fold (p<0.0001) reductions, respectively. Further 5′ deletions, to generate Prm1F-1J (data not shown), in combination with bioinformatic analysis to identify elements within the −6320 to −5895 region revealed five putative overlapping sites for Sp1/Egr1 and a putative NF-E2/AP1 site (FIG. 3). Hence, site-directed mutagenesis was used to disrupt those putative Sp1/Egr1 and NF-E2/AP1 sites within either Prm1HΔ (−6320) or Prm1L (−6010). Mutation of the Sp1/Egr1⁻⁶⁰⁰⁷ site within Prm1L significantly reduced, but did not abolish, luciferase expression (FIG. 3A, p=0.0135). Mutation of four of the five Sp1/Egr1 sites, specifically Sp1/Egr1⁻⁶²⁹⁴, Sp1/Egr1⁻⁶²⁷⁸, Sp1/Egr1⁻⁶⁰²² and Sp1/Egr1⁻⁶⁰⁰⁷, but not Sp1/Egr1⁻⁶⁰⁹⁸, each reduced luciferase activity directed by Prm1H□ (p=0.0096, p=0.0005, p<0.0001, p<0.0001, respectively; FIG. 3B). Furthermore, disruption of the putative NF-E2/AP1⁻⁶⁰⁸⁰ site also reduced luciferase activity directed by Prm1HΔ (p<0.0001). Thereafter, to investigate possible cooperative actions of the latter, the effect of mutating combinations of the Sp1/Egr1 and NF-E2/AP1 elements within Prm1HΔ was examined (FIG. 3C & data not shown). As stated, disruption of Sp1/Egr1⁻⁶²⁹⁴ and Sp1/Egr1⁻⁶⁰²² both decreased luciferase expression directed by Prm1HΔ, where disruption of Sp1/Egr1⁻⁶⁰²² caused a more pronounced decrease (1.8-fold; p<0.0001) than mutation of Sp1/Egr1⁻⁶²⁹⁴ (1.3-fold; p=0.0096). Mutation of both elements together, generating Prm1HΔ^{Sp1/Egr1(-6294,-6022)}, also decreased luciferase expression compared to that of Prm1HΔ (p<0.0001). However, the magnitude of this decrease (1.8-fold) was not greater than of Sp1/Egr1⁻⁶⁰²² alone. Furthermore, the activity directed by Prm1HΔ^{Sp1/Egr1 (-6294,-6022)}* was not significantly different from that of Prm1HΔ^{Sp1/Egr1(-6022)}* (p=0.5033). Similarly, mutation of Sp1/Egr1⁻⁶⁰²² and Sp1/Egr1⁻⁶⁰⁰⁷ both led to decreased luciferase expression directed by Prm1HΔ (1.8-fold; p<0.0001 and 1.5-fold; p<0.0001, respectively). Disruption of both elements, generating Prm1HΔ^{Sp1/Egr1(-6022,-6007)}* reduced luciferase expression relative to that of Prm1HΔ (p=0.0001). The extent of this decrease (1.5-fold) was of the same order as that caused by Sp1/Egr1⁻⁶⁰⁰⁷. Moreover, the luciferase activity directed by Prm1HΔ^{SP1/Egr1(-6022,-6007)}* was not significantly different from that of Prm1HΔ^{SP1/Egr1(-6022)}* (p=0.1333) or Prm1HΔ^{SP1/Egr1(-6007)}* (p 0.7571). Hence, collectively, these and other combinations of mutations (data not shown) indicate that the Sp1/Egr1 and/or NF-E2/AP1 elements within the −6320 to −5895 region act interdependently and functionally cooperate to regulate Prm1. Thereafter, electrophoretic mobility shift assays (EMSAs) were carried out to investigate the presence and identity of nuclear factors capable of binding to the NF-E2/AP1⁻⁶⁰⁸⁰ element in vitro (FIG. 4A). Expression of NF-E2 (FIG. 4C) and the AP1 component cJun (data not shown) in HEL 92.1.7 cells was confirmed by immunoblot analysis. Incubation of a biotin-labelled NF-E2/AP1 probe with nuclear extract from HEL cells resulted in the appearance of a main protein-DNA complex, C1, as well as one or more faster-migrating complexes (FIG. 4A, lane 2). The main C1 complex was competed by specific NF-E2/AP1⁻⁶⁰⁸⁰ or consensus NF-E2 sequences but not by a consensus AP1 sequence (FIG. 4A, lanes 3-5). It appears that the faster-migrating complexes were competed in a similar manner to C1. Following prolonged exposure of the chromatogram in FIG. 4A, a further slower-migrating complex, designated C2, and equivalent to C2 in FIG. 3B, was observed and, like C1, was competed by NF-E2/AP1⁻⁶⁰⁸⁰ and consensus NF-E2 sequences but not by the consensus AP1 sequence (data not shown). Thereafter, pre-incubation of nuclear extract with an anti-NF-E2 antibody resulted in a supershifted complex (FIG. 4B). However, it appeared that C1 was not significantly reduced following formation of this supershift, suggesting that the supershifted NF-E2 may have originated from a complex other than C1. While no supershift was observed with an anti-cJun antibody, it appeared that addition of this antibody reduced both C1 and C2, suggesting a possible role for cJun binding to the NF-E2/AP1 probe (FIG. 4B, lane 4). To investigate whether NF-E2 can directly bind to Prm1 in vivo, chromatin immunoprecipitation (ChIP) assays were carried out on chromatin extracted from HEL cells (FIG. 4D). PCR analysis using primers specific to the 3′ Prm1 region (−6368 to −5895) generated amplicons from both the input chromatin and from an anti-NF-E2, but not from a control IgG, immunoprecipitate (FIG. 4D). PCR analysis using primers specific to the −8460 to −8006 region of Prm1, which does not contain any predicted NF-E2 elements, resulted in generation of an amplicon from input chromatin, but not from anti-NF-E2 nor IgG precipitates (FIG. 4E). Taken together, EMSA and supershifts demonstrate that NF-E2 specifically binds to the NF-E2/AP1⁻⁶⁰⁸⁰ probe in vitro, while ChIP assays establish that NF-E2 occupies element(s) within the −6368 to −5895 region of Prm1 in vivo. EMSAs also investigated nuclear factor binding to the Sp1/Egr1⁻⁶²⁹⁴ and Sp1/Egr1⁻⁶²⁷⁸ elements in vitro. Immunoblot analysis confirmed abundant expression of Sp1 and Egr1 in HEL cells (FIGS. 5A & 5B). Incubation of the Sp1/Egr1⁻⁶²⁹⁴ probe with nuclear extract generated two DNA-protein complexes, C1 and C2 (FIG. 5C, lane 2). Both C1 and C2 were efficiently competed by the Sp1/Egr1⁻⁶²⁹⁴ and consensus Egr1 sequences (FIG. 5C, lanes 3 & 5, respectively), and to a lesser extent by consensus Sp1 and WT-1 sequences (FIG. 5C, lanes 4 & 6, respectively). Neither C1 nor C2 were competed by a non-specific randomized sequence based on the TP gene (FIG. 5C, lane 7). Moreover, addition of an anti-Egr1 antibody resulted in generation of a supershift complex, as well as reducing both C1 and C2 (FIG. 5D, lane 4). While no supershift was observed with an anti-Sp1 antibody, both C1 and C2 were substantially reduced following its addition (FIG. 5D, lane 3), indicating a possible role for Sp1 binding to the probe. Addition of an anti-WT-1 antibody or an anti-cJun antibody, used as a control, had no substantial effects on binding patterns to the probe (FIG. 5D, lanes 5 & 6, respectively). Collectively, these data indicate that complexes of Sp1 and Egr1 from HEL cell nuclear extract can bind to the Sp1/Egr1⁻⁶²⁹⁴ element within Prm1 in vitro. Thereafter, EMSAs were carried out to investigate the presence of nuclear factors capable of binding to the Sp1/Egr1⁻⁶²⁷⁸ element in vitro. Incubation of the Sp1/Egr1⁻⁶²⁷⁸ probe with HEL cell nuclear extract generated one main complex, designated C1 (FIG. 5E, lane 2). C1 was efficiently competed by Sp1/Egr1⁻⁶²⁷⁸, consensus Sp1 and consensus Egr1 sequences, and to a much lesser extent by the WT-1 sequence (FIG. 5E, lanes 3-6, respectively). C1 was not competed by a non-specific randomized sequence based on the TP gene (FIG. 5E, lane 7). Moreover, addition of an anti-Egr1 antibody generated a supershift complex, as well as reducing the main complex C1. While addition of an anti-Sp1 antibody did not lead to observation of a supershift complex, it reduced C1 in a similar manner to the anti-Egr1 antibody, indicating a possible role for Sp1 binding. Addition of an anti-WT-1 antibody or an anti-cJun antibody, used as a control, did not have any substantial effects on binding patterns to the probe. Collectively, these data indicate that a complex of Sp1 and Egr1 from HEL cell nuclear extract can bind to the Sp1/Egr1⁻⁶²⁷⁸ element within Prm1 in vitro. EMSAs also confirmed the presence of nuclear factors capable of binding to the Sp1/Egr1⁻⁶⁰²² and Sp1/Egr1⁻⁶⁰⁰⁷ elements in vitro. Incubation of the Sp1/Egr1^{-6022,-6007} probe with HEL cell nuclear extract resulted in two main complexes, C1 and C2 (FIG. 6A). Both C1 and C2 were competed by both Sp1/Egr1⁻⁶⁰²² and Sp1/Egr1⁻⁶⁰⁰⁷ specific sequences (FIG. 6A, lanes 3-5, respectively). The faster migrating C1 complex was efficiently competed by consensus Sp1, consensus Egr1 and WT-1 sequences (FIG. 6A, lanes 6-8, respectively). It was notable, however, that C2 was actually increased by consensus Sp1, Egr1 or WT-1 oligonucleotides (FIG. 6A, lanes 6-8, respectively), suggesting that nuclear factor(s) other than Sp1, Egr1 or WT-1 may possibly bind to the Sp1/Egr1^{-6022,-6007} probe in vitro, and that these factor(s) may bind more efficiently to the probe when the protein(s) involved in the formation of C1 are unavailable for binding. A non-specific randomized sequence based on the TP gene (FIG. 6A, lane 9) failed to inhibit C1 or C2. These data indicate that Sp1, Egr1 and/or WT-1 proteins from HEL cells bind to the sites at −6022 and −6007 within Prm1. Moreover, anti-Sp1 (FIG. 6B left, lane 3) and anti-Egr1 (FIG. 6B right, lane 4) antibodies both resulted in supershift complexes while no supershifts were observed with either anti-WT-1 or, as a control, anti-cJun antibodies, even following prolonged exposure of the chromatogram (FIG. 6B, lanes 5 and 6, respectively). Due to the weak nature of the supershifted complexes observed following pre-incubation with either anti-Sp1 or anti-Egr1 antibodies, it was not clear whether the Sp1 or Egr1 in the supershifted complexes actually originated from C1. Thereafter, in order to further investigate the possible binding of Sp1 and Egr1 to the proximal Prm1, ChIP analysis was carried out. Primers based on the proximal Prm1 region generated amplicons from both anti-Sp1 and anti-Egr1, but not from the control IgG, immunoprecipitates (FIG. 6C). Conversely, PCR analysis using primers specific to an upstream region of Prm1 from −8460 to −8006 resulted in generation of an amplicon from input chromatin, but not from anti-Sp1, anti-Egr1 nor IgG precipitates (FIG. 6D). Additionally, over-expression of Egr1 led to a modest, but significant, decrease in the luciferase expression directed by Prm1HΔ in HEL cells (FIG. 6E). Hence, collectively, four overlapping Sp1/Egr1 sites and a NF-E2 site have been identified in the proximal Prm1 region. Both Sp1 and Egr1, in addition to NF-E2, bind to those elements in vitro and in vivo to regulate core Prm1 while over-expression of Egr1 appears to negatively regulate that transcriptional activity.

### Example 3

### Identification of Functional GATA and Ets Sites within Prm1

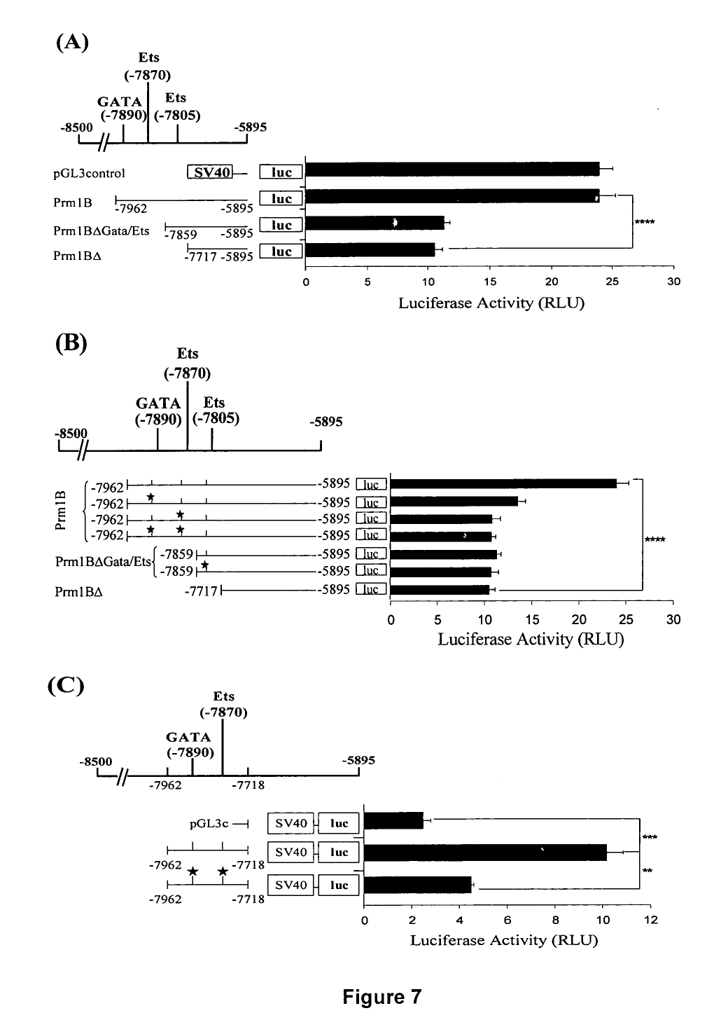

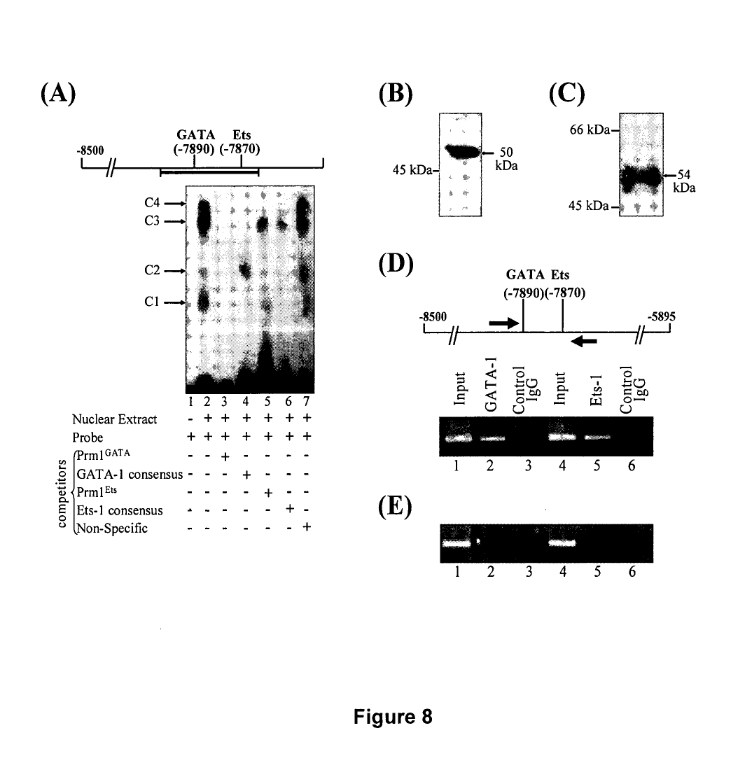

As stated, 5′ deletions of Prm1 revealed an UAS between −7962 (Prm1B) and −7717 (Prm1BΔ), deletion of which yielded a 2.8-fold reduction in luciferase expression, FIG. 2A). To localise the regulatory element(s) within this region, an additional 5′ sub-fragment Prm1BΔGata/Ets (−7859) was generated. Removal of nucleotides between −7962 (Prm1B) and −7859 (Prm1BΔGata/Ets) led to a 2.3-fold reduction in luciferase activity (FIG. 7A; p<0.0001), while there was no difference in expression between Prm1BΔGata/Ets and Prm1B [symbol] (p=0.261). Amongst the transcription factor elements identified between −7962 and −7859 were putative GATA and Ets elements at −7890 and −7870, respectively. Mutation of GATA⁻⁷⁸⁹⁰ and Ets⁻⁷⁸⁷⁰ elements both reduced luciferase expression directed by Prm1B (FIG. 7B), where the decrease due to the Ets⁻⁷⁸⁷⁰ mutation (2.2-fold; p<0.0001) was more pronounced than that caused by the GATA⁻⁷⁸⁹⁰ mutation (1.8-fold; p<0.0001). Luciferase expression directed by Prm1B^{GATA (-7890)}*^{,Ets(-7870)}*, where both GATA⁻⁷⁸⁹⁰ and Ets⁻⁷⁸⁷⁰ were mutated, was also significantly lower than that of the wild-type Prm1B. However, the magnitude of this decrease (2.2-fold) was not greater than that caused by the Ets⁻⁷⁸⁷⁰ mutation alone. Moreover, luciferase activity directed by Prm1B^{GATA(-7890)}*^{,Ets(-7870)}* was not significantly different to that directed by Prm1B^{Ets(-7870)}* (p=0.9293; FIG. 7B). Collectively, these single and combination mutations suggest that the GATA⁻⁷⁸⁹⁰ and Ets⁻⁷⁸⁷⁰ elements do not act independently but rather, cooperatively in an interdependent manner. Although, it was already established (FIG. 7B) that there was no difference in luciferase expression directed by Prm1BΔGata/Ets (−7859) and Prm1 BΔ (−7717), a second putative Ets site, namely Ets⁻⁷⁸⁰⁵, was identified adjacent to the aforementioned GATA⁻⁷⁸⁹⁰ and Ets⁻⁷⁸⁷⁰ sites. However, site-directed mutagenesis of the latter Ets⁻⁷⁸⁰⁵ element did not significantly affect the level of luciferase activity (FIG. 7B; p=0.4287). Hence, collectively these data suggest that GATA⁻⁷⁸⁹⁰ and Ets⁻⁷⁸⁷⁰ elements act as upstream activators of Prm1 and may functionally cooperate to positively regulate basal Prm1 in HEL cells, while the putative Ets⁻⁷⁸⁰⁵ element does not appear to be functional. To investigate the ability of the UAS encoding GATA⁻⁷⁸⁹⁰ and Ets⁻⁷⁸⁷⁰ elements to regulate general gene expression in HEL cells, a Prm1 sub-fragment spanning −7962 to −7718 was placed upstream of the heterologous SV40 promoter in the plasmid pGL3Control. The Prm1GATA/Ets sub-fragment resulted in a 4.1-fold increase in luciferase expression relative to that of the SV40 promoter alone (FIG. 7C; p=0.0003). Moreover, the level of luciferase expression directed by the Prm1GATA/Ets variant, in which both the GATA⁻⁷⁸⁹⁰ and Ets⁻⁷⁸⁷⁰ elements were mutated, was significantly impaired (p=0.0012), resulting in only a 1.8-fold increase in SV40-directed luciferase activity (FIG. 7C, p=0.0003). These data indicate that the Prm1 region from −7962 to −7718 acts as an UAS, greatly increasing the activity of the SV40 promoter in HEL cells, an effect mainly attributable to the GATA⁻⁷⁸⁹⁰ and Ets⁻⁷⁸⁷⁰ cis-acting elements. Thereafter, EMSAs explored the presence of nuclear factors capable of binding to the GATA⁻⁷⁸⁹⁰ and Ets⁻⁷⁸⁷⁰ elements in vitro. Immunoblot analysis confirmed expression of both GATA-1 and Ets-1 in HEL cells (FIGS. 8B & 8C). Incubation of a GATA/Ets probe with nuclear extract prepared from HEL cells generated four DNA-protein complexes, C1-C4 (FIG. 8A, lane 2). C2 was competed by either the Prm1 GATA⁻⁷⁸⁹⁰ or Ets⁻⁷⁸⁷⁰ sequences, as well as by a consensus Ets-1, but was not competed by a consensus GATA-1 sequence (FIG. 8A, lane 3-6). These data indicate that C2 consists of Ets-1 and another factor bound to the GATA/Ets probe. C3 was competed by GATA⁻⁷⁸⁹⁰ and consensus GATA-1, but not by Ets⁻⁷⁸⁷⁰ or consensus Ets-1 sequences (FIG. 8A, lanes 3-6, respectively), suggesting that C3 consists of GATA-1 protein, possibly complexed with another factor, bound to the GATA/Ets probe. Complexes C1 and C4 were competed by either GATA⁻⁷⁸⁹⁰ or consensus GATA-1 sequences, as well as by Ets⁻⁷⁸⁷⁰ and consensus Ets-1 sequences (FIG. 8A, lanes 3-6, respectively). The non-specific competitor, based on a randomized TP gene failed to inhibit any of the C1-C4 complexes (FIG. 8A, lane 7), confirming the specificity of the GATA/Ets probe. Therefore, complexes of GATA-1 and Ets-1 proteins from HEL cell nuclear extract can bind to Prm1 GATA⁻⁷⁸⁹⁰ and Ets⁻⁷⁸⁷⁰ elements in vitro. Moreover, ChIP assays confirmed the specific amplification of the Prm1 proximal region from anti-GATA-1 and anti-Ets-1 immunoprecipitates, but not from the control IgG precipitate (FIG. 8D), confirming that both GATA-1 and Ets-1 occupy element(s) within the −7978 to −7607 region of Prm1 in vivo. Conversely, primers specific to the proximal region of Prm1 from −6368 to −5895 resulted in generation of an amplicon from the input chromatin, but not from the GATA-1, Ets-1 or control IgG precipitates (FIG. 8E).

### Example 4

### Identification of Three Distinct Repressor Regions within Prm1 of the Human TP Gene

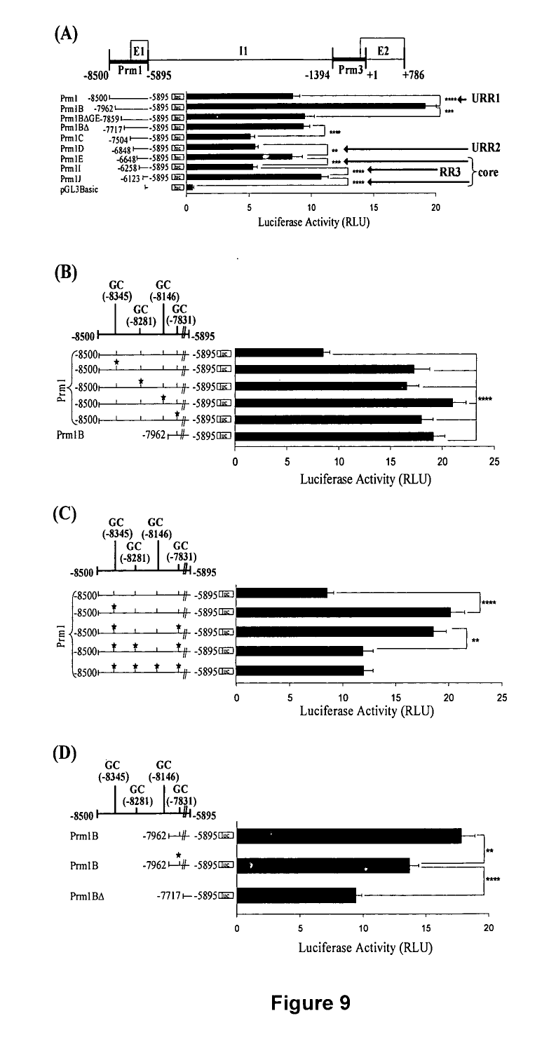

The TPα and TPβ isoforms of the TXA₂ receptor (TP) are under the transcriptional regulation of Prm1 and Prm3, respectively, within the human TP gene (Coyle et al., 2002). Prm1 is defined as nucleotides −8500 to −5895 located 5′ of the translation initiation codon (Coyle et al., 2002). Sp1, Egr1 and NF-E2 have been identified as the key trans-acting factors that regulate the “core” proximal Prm1 (−6320 to −5895), while two upstream activator regions (UAR) and two upstream repressor regions (URR) have also been identified (FIG. 9A). Additionally, GATA-1 and Ets-1 have been identified as the key factors that bind and regulate UAR1 (−7962 to −7717). Conversely, the factors regulating UAR2 (−7717 to −7504), as well as URR1 (−8500 to −7962) and URR2 (−6848 to −6648) remain to be identified (FIG. 9A). Initially, genetic reporter assays and progressive 5′ deletion of nucleotides from −8500 to −6648 to yield the core promoter (Prm1E; FIG. 9A) confirmed the presence of two upstream regions of repression, namely URR1 and URR2. Specifically, deletion of nucleotides from Prm1 (−8500) to generate Prm1B (−7962) yielded a 2.3-fold increase in luciferase expression (p<0.0001), while 5′ deletion of Prm1D (−6848) to generate Prm1E (−6648) resulted in a 1.5-fold increase in luciferase activity (p=0.0032). Further 5′ deletion of Prm1E (−6648) to generate Prm1I (−6258) resulted in a 1.6-fold decrease in luciferase expression (p=0.0002), thereby uncovering an activator region within the core promoter region. Consistent with this observation, two functional overlapping Sp1/Egr1 elements have now been identified within this region, specifically at −6294 and −6278, that mediate activation of Prm1. However, herein, further 5′ deletion of Prm1I (−6258) to generate Prm1J (−6123) yielded a 2-fold increase in luciferase activity (p<0.0001) to reveal a third, previously unidentified, repressor region (−6258 to −6123) also located within the “core” Prm1. The luciferase expression directed by Prm1J was substantially higher than that of the empty pGL3Basic vector (FIG. 9A). Consistent with this observation, two functional overlapping Sp1/Egr1 elements within Prm1J, specifically at −6022 and −6007, in addition to an NF-E2 element at −6080 have now been identified, and these elements mediate activation of Prm1 within this core proximal region. Hence, collectively, three distinct regions of repression have been identified within Prm1, namely the two previously identified URR1 (−8500 to −7962) and URR2 (−6848 to −6648) and an additional repressor region, designated RR3, located between −6258 and −6123 within the proximal core promoter.

### Example 5

### Identification of Multiple GC-Enriched Elements in the −8500 to −7962 Repressor Region of Prm1

Bioinformatic analysis (Quandt et al., 1995) to identify transcription factor elements within URR1 located between −8500 and −7962 revealed three putative GC elements representing putative overlapping WT1/Egr1/Sp1 sites at −8345, −8281 and −8146, where the 5′ nucleotide of each element is indicated (Table 2 and FIG. 9B). Site-directed mutagenesis (SDM) of each of these GC elements led to substantial increases in luciferase activity directed by Prm1 (2.0-fold, 1.9-fold, and 2.5-fold, respectively; p<0.0001 in each case; FIG. 9B). A fourth GC element was identified somewhat adjacent to URR1, specifically at −7831 within Prm1B. Mutation of this element also substantially increased luciferase activity directed by Prm1 (2.1-fold; p<0.0001). These data suggest that the GC elements at −8345, −8281, −8146 and −7831 mediate repression of Prm1.

Table 2: Consensus sequences for Egr1, WTE and Sp1, as well as sequences of GC elements within Prm1. Base pairs underlined denote the core sequences of the elements, while base pairs in capital letters are in positions that exhibit a high conservation profile (Quandt et al., 1995). The + and − designation indicates that elements are found on the sense or antisense strands of Prm1, respectively.

| | | SEQ |
|---|---|---|
| Element | Sequence | ID NO |
| Egr1 | 5′ GcGGGGGCG 3′ | |
| consensus | | |
| WTE | 5′ gtgcGTGGGaGtagaat 3′ | 116 |
| consensus | | |
| Sp1 | 5′ gGGGCGGGgc 3′ | 117 |
| consensus | | |
| Prm1⁻⁸³⁴⁵ (−) | 5′ ctggGTGGGGGCGGGgGcagctt 3′ | 118 |
| Prm1⁻⁸²⁸¹ (−) | 5′ tccgGcGGGGGCCGGgcag 3′ | 119 |
| Prm1⁻⁸¹⁴⁶ (+) | 5′ ggcGGGGGGTGGGGGGCGGGGGGC | 120 |
| | GGGccaa 3′ | |
| Prm1⁻⁷⁸³¹ (−) | 5′ agatGaGGGGGCAgtga 3′ | 121 |
| Prm1⁻⁶⁷¹⁷ (−) | 5′ ccagGGGTGGGGTGGGaGgacaga 3′ | 122 |
| Prm1⁻⁶²⁰⁶ (−) | 5′ acggGTGGGgGccgctg 3′ | 123 |

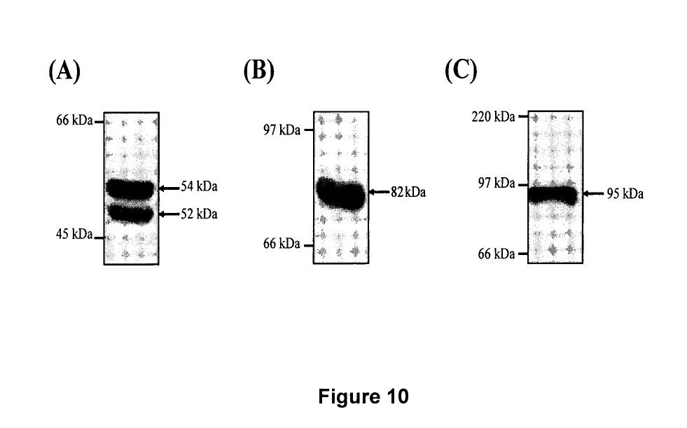

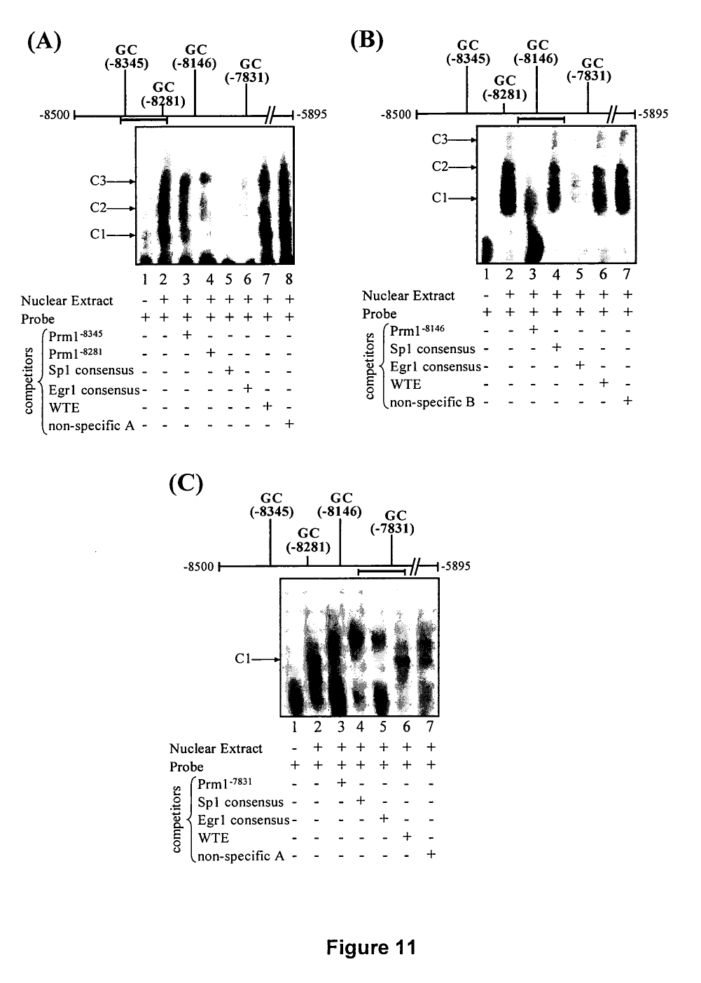

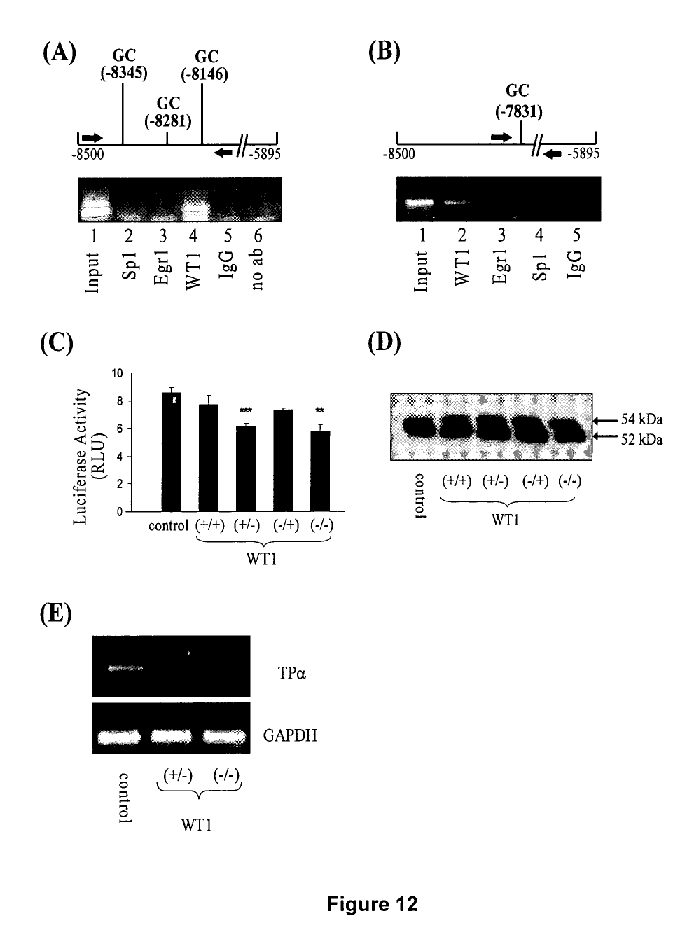

To investigate the combined contribution of GC elements in directing Prm1 activity, the effect of collectively mutating the sites within Prm1 (−8500) was examined (FIG. 9C). Disruption of the GC⁻⁷⁸³¹ element within Prm1^{GC}*⁽⁻⁸³⁴⁵⁾ to generate Prm1^{GC}*^{(-8345,-7831)} did not significantly affect luciferase expression directed by Prm1^{GC}*⁽⁻⁸³⁴⁵⁾ (p=0.3781). However, disruption of the GC⁻⁸²⁸¹ element in Prm1^{GC}*^{(-8345,-7831)}, generating Prm1^{GC}*^{(-8345,-8281,-7831)}, yielded a 1.6-fold decrease in luciferase expression compared to that of Prm1^{GC}*^{(-8345,-7831)} (p 0.0043). Luciferase expression of Prm1^{GC}*^{(-8345,-8281,-8146,-7831)} was not significantly different than that of Prm1^{GC}*^{(-8345,-8281, -7831)} (p=0.7499). Therefore, generation of Prm1^{GC}*^{(-8345,-8281,-8146,-7831)} from Prm1^{GC}*⁽⁻⁸³⁴⁵⁾ led to an overall 1.7-fold decrease in luciferase expression (p=0.0024), suggesting that repressor factors rely on a cooperative mechanism of binding to multiple neighbouring GC elements. It is likely that disruption of cooperative binding upon SDM shifts the overall affinity of intact GC elements for activator, rather than for repressor factors. Consistent with this suggestion, disruption of GC⁻⁷⁸³¹ in the Prm1B (−7962) sub-fragment, which does not contain any of the other three GC elements, actually decreased the luciferase activity directed by Prm1B (1.4-fold; p=0.0004; FIG. 9D). This effect is in contrast to the substantial increase (2.1-fold; p<0.0001; FIG. 9B) in luciferase expression that occurred upon disruption of the same GC element within Prm1 where the other three GC elements at −8345, −8281 and −8146 were intact (compare FIGS. 9B and 9D). Thereafter, electrophoretic mobility shift assays (EMSAs) were carried out to investigate the presence and identity of nuclear factors capable of binding to the overlapping WT1/Egr1/Sp1 elements at −8345, −8281, −8146 and −7831 in vitro (FIG. 11). Herein, the expression of WT1, Sp1 and Egr1 in the HEL 92.1.7 cell line was initially confirmed by immunoblot analysis (FIG. 10). A doublet of WT1 protein at 52/54 kDa was detected herein in HEL cells (FIG. 10A), and an immunoreactive Egr1 band of approximately 82 kDa was detected in HEL cells (FIG. 10B), while abundant expression of the ubiquitous Sp1 protein (95 kDa) was also confirmed (FIG. 10C). Incubation of biotin-labelled oligonucleotide probes encoding GC⁻⁸³⁴⁵ and GC⁻⁸²⁸¹ (FIG. 11A), GC⁻⁸¹⁴⁶ (FIG. 11B) and GC⁻⁷⁸³¹ (FIG. 11C) with nuclear extract prepared from HEL cells resulted in the appearance of a number of protein-DNA complexes. Specifically, incubation of the probe encoding both GC⁻⁸³⁴⁵ and GC⁻⁸²⁸¹ elements with nuclear extract generated three main complexes, C1-C3 (FIG. 11A). C1 and C2 were partially competed by non-labelled competitors containing either the specific GC⁻⁸³⁴⁵, GC⁻⁸²⁸¹, consensus Sp1 or Egr1 sequences (FIG. 11A, lanes 3-6, respectively. C3 was efficiently competed by consensus Sp1 or Egr1 sequences but not by GC⁻⁸³⁴⁵ or GC⁻⁸²⁸¹ sequences. The WTE sequence or a non-specific competitor based on a random sequence within the TP gene failed to compete with C1, C2 or C3 complexes (FIG. 11A, lanes 7 and 8, respectively). Therefore, it seems that complexes C1 and C2 consist of Sp1, Egr1 and/or WT1 proteins bound to the GC⁻⁸³⁴⁵ and GC⁻⁸²⁸¹ elements in vitro, whilst C3 may consist of Sp1, Egr1 and/or WT1 bound to an unidentified element within the probe (FIG. 11A). Incubation of the GC⁻⁸¹⁴⁶ probe with nuclear extract generated three main complexes, C1-C3 (FIG. 11B). All three complexes were efficiently competed by non-labelled competitors containing the GC⁻⁸¹⁴⁶ or consensus Egr1 sequences, while none of the three complexes were competed by the consensus Sp1, WTE or non-specific oligonucleotide sequences. Thus, it is indicated that complexes C1-C3 consist of Egr1 and/or WT1 protein, possibly complexed with other factor(s), bound to the GC⁻⁸¹⁴⁶ element (FIG. 11B). Incubation of the GC⁻⁷⁸³¹ probe with nuclear extract generated a single complex C1 (FIG. 11C) that was competed by a non-labelled competitor containing the GC⁻⁷⁸³¹ element, or by consensus Sp1 or Egr1 sequences, but not by WTE or non-specific sequences. Therefore, C1 is likely to consist of Sp1, Egr1 and/or WT1 proteins complexed to the GC⁻⁷⁸³¹ element. It was notable, however, that a second slower-migrating complex appeared where the main complex C1 was competed by non-labelled competitors. Thus, it is possible that unidentified protein(s) can bind to an element within the probe when the protein(s) involved in the formation of C1 are unavailable for binding. Overall, these EMSA data (FIG. 11) indicate that GC⁻⁸³⁴⁵, GC⁻⁸²⁸¹, GC⁻⁸¹⁴⁶ and GC⁻⁷⁸³¹ elements have a sequence capacity to bind Egr1 and/or WT1 isoform(s), while GC⁻⁸³⁴⁵, GC⁻⁸²⁸¹ and GC⁻⁷⁸³¹ also have a capacity to bind Sp1. Thereafter, to investigate whether endogenous Sp1, Egr1 and/or WT1 can actually directly bind to chromatin encoding Prm1 in vivo, chromatin immunoprecipitation (ChIP) assays were carried out on chromatin extracted from HEL cells (FIG. 12). PCR analysis using primers to amplify the 5′ Prm1 repressor region, specifically from −8460 to −8006 and containing GC elements at −8345, −8281 and −8146, resulted in amplification of DNA recovered from both the input chromatin and from an anti-WT1 immunoprecipitate, but not from anti-Sp1, anti-Egr1 or control IgG precipitates (FIG. 12A). Furthermore, PCR analysis using primers specific to the adjacent Prm1 region, specifically from −7978 to −7607 and containing the GC element at −7831, also resulted in amplification of DNA recovered from the input chromatin and an anti-WT1 immunoprecipitate, but not from anti-Sp1, anti-Egr1 or control IgG precipitates (FIG. 12B). These data provide evidence that WT1, but not Sp1 nor Egr1, occupies element(s) within the Prm1 −8460 and −7607 region of chromatin in HEL cells in vivo. Hence, to expand these studies, the effect of ectopic expression of WT1 on Prm1-directed reporter gene expression and TPα mRNA was investigated (FIGS. 12C-12E). The four main isoforms of WT1, specifically (+/+), (+/−), (−/+) and (−/−) with respect to the presence or absence of exon 5 and KTS sequences, respectively, were over-expressed in HEL cells (FIG. 12D) and the effects on Prm1-directed luciferase activity were investigated. The (+/−) and (−/−) isoforms led to 1.4-fold (p=0.0009) and 1.5-fold (p=0.0022) reductions in Prm1-directed luciferase expression, respectively, while neither the (+/+) nor (−/+) isoforms had any significant effect (p=0.0612 and p=0.3133; FIG. 12C). Consistent with this, RT-PCR confirmed that ectopic expression of the transcriptionally active (+/−) and (−/−) isoforms both significantly reduced TPα mRNA expression (FIG. 12E). No substantial changes in GAPDH expression were observed following over-expression of WT1 isoforms (FIG. 12E). Taken together, these data indicate that −KTS isoforms of WT1 mediate repression of Prm1 and TPα expression and considering the data from mutational, EMSA and ChIP analyses, it appears that WT1 exerts this repression by binding to GC elements at −8345, −8281, −8146 and −7831.

### Example 6

### Identification of GC Elements in the −6848 to −6648 and −6258 to −6123 Repressor Regions of Prm1

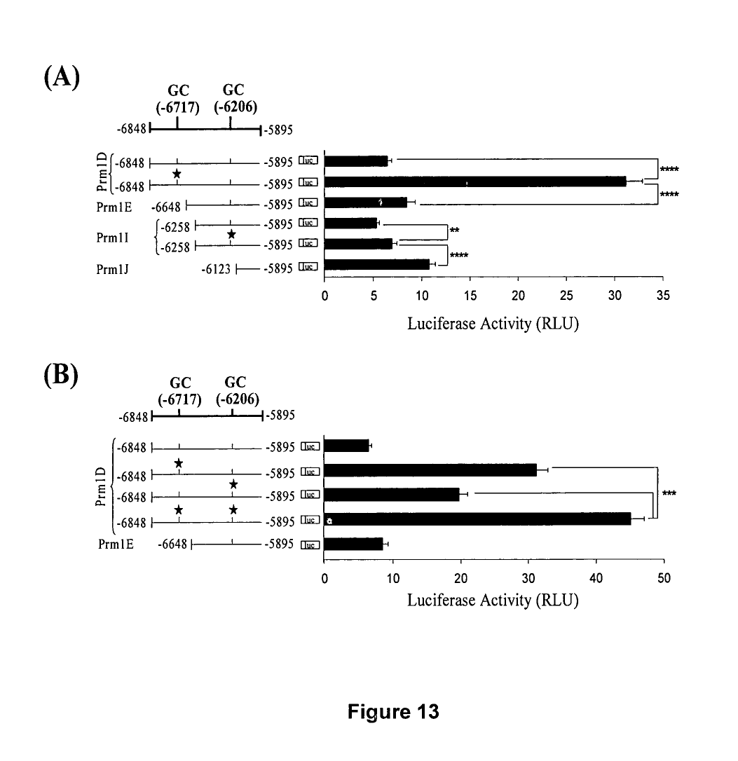

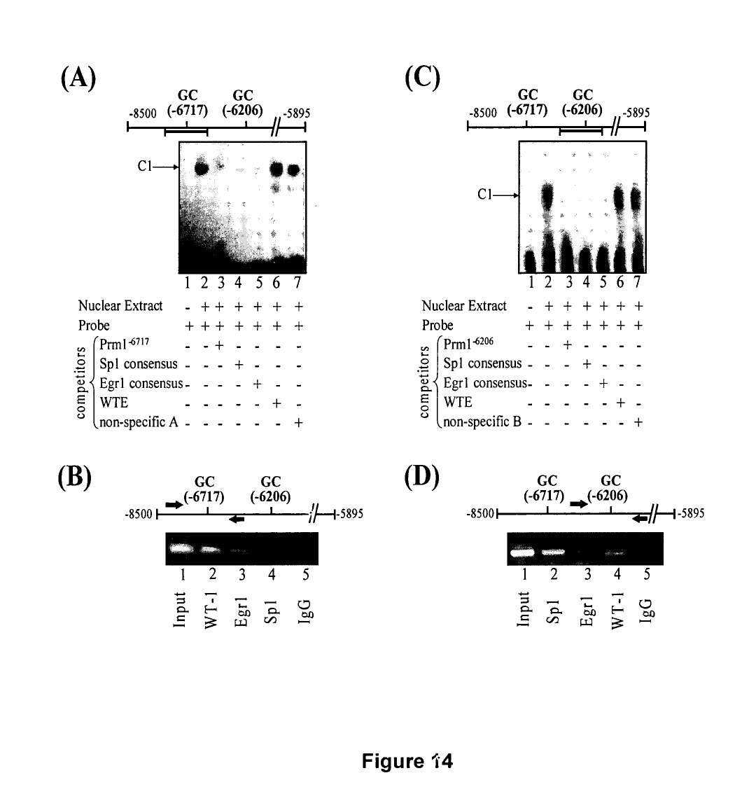

Amongst the transcription factor binding elements identified within URR2 located between −6848 and −6648 (FIG. 9A), was a putative GC element at −6717 predicted to represent a putative overlapping site for WT1/Egr1/Sp1. Additionally, bioinformatic analysis of the “core” repressor region, from −6258 to −6123 of Prm1, also revealed a GC element, specifically at −6206 (Table 2 above). Hence, SDM was used to disrupt the putative GC⁻⁶⁷¹⁷ and GC⁻⁶²⁰⁶ elements within either Prm1D (−6848) or Prm1I (−6258; FIG. 13). Mutation of the GC⁻⁶⁷¹⁷ element within Prm1D resulted in a 4.8-fold increase in luciferase expression compared to that of the wild-type Prm1D (p<0.0001). Mutation of the GC⁻⁶²⁰⁶ element within Prm1I led to a 1.3-fold increase in luciferase expression (p=0.0083; FIG. 13A). Mutation of the same GC⁻⁶²⁰⁶ element within the Prm1D sub-fragment led to a 3-fold increase in luciferase activity compared to that of the wild-type Prm1D (p<0.0001; FIG. 13B). To investigate the combined contribution of GC⁻⁶⁷¹⁷ and GC⁻⁶²⁰⁶ elements in directing Prm1 activity, the effect of collectively mutating these elements within Prm1D was examined (FIG. 13B). The luciferase activity directed by Prm1D^{GC}*^{(-6717,-6206)}, in which both GC elements at −6717 and −6206 were mutated, was significantly higher than that of either Prm1D^{GC}*⁽⁻⁶⁷¹⁷⁾, in which the −6717 element alone was mutated, or Prm1D^{GC}*⁽⁻⁶²⁰⁶⁾, in which the −6206 element alone was mutated (p<0.0001 in each case). Hence, collectively, these data indicate that GC elements at −6717 and −6206 bind factors that act independently to mediate repression of Prm1. Thereafter, EMSAs were employed to confirm the presence of nuclear factors capable of binding to the GC⁻⁶⁷¹⁷ element in vitro (FIG. 14A). Incubation of a GC⁻⁶⁷¹⁷ probe with nuclear extract prepared from HEL cells generated a single DNA-protein complex, C1 (FIG. 14A). C1 was efficiently competed by a non-labelled competitor containing the GC⁻⁶⁷¹⁷ sequence, and by consensus Sp1 or Egr1 sequences, but was not competed by WTE or non-specific sequences. Thus, C1 consists of complexes of Sp1, Egr1 and/or WT1 proteins bound to the GC⁻⁶⁷¹⁷ probe. To investigate whether Sp1, Egr1 and/or WT1 can bind to chromatin encoding −6848 to −6648 region of Prm1 in vivo, ChIP assays were carried out using chromatin extracted from HEL cells (FIG. 14B). PCR analysis using primers specific to this region of Prm1 and containing the GC element at −6717 resulted in generation of amplicons from input chromatin, anti-WT1 and to a lesser extent anti-Egr1 immunoprecipitates, but not from anti-Sp1 or the control IgG precipitates. These data provide evidence that WT1, and to a lesser extent, Egr1 occupy element(s) within the −6848 to −6648 region of Prm1 in vivo. EMSAs were also employed to investigate the presence of nuclear factors capable of binding to the GC⁻⁶²⁰⁶ element in vitro (FIG. 14C). Incubation of a GC⁻⁶²⁰⁶ probe with nuclear extract generated a single diffuse complex C1 that was efficiently competed by a non-labelled competitor containing the GC⁻⁶²⁰⁶ sequence, or by consensus Sp1 or Egr1 sequences, but not by WTE or non-specific sequences. Thus, the complex consists of Sp1, Egr1 and/or WT1 proteins bound to the GC⁻⁶²⁰⁶ probe. To investigate whether Sp1, Egr1 and/or WT1 can bind to the proximal Prm1 (from −6320 to −5895) in vivo, ChIP assays were carried out (FIG. 14D). PCR generated amplicons consisting of Prm1 sequences between −6368 and −5895 from input chromatin, anti-WT1, anti-Sp1 and anti-Egr1 immunoprecipitates but not from the control IgG precipitate. It has been previously established that both Sp1 and Egr1 bind to this region (−6368 to −5895) of Prm1 in vivo, where binding was established to occur at overlapping Sp1/Egr1 elements at −6294, −6278, −6022 and −6007 within the proximal Prm1. Hence, evidence is also presented herein that WT1 binds to the proximal Prm1 in vivo, and the binding of WT1 is likely to occur at the GC element at −6206. Thereafter, the effects of ectopic expression of WT1 on luciferase activity directed by Prm1D (−6848) and Prm1I (−6258) were investigated (data not shown). The (+/+), (+/−), (−/+) and (−/−) isoforms of WT1 were over-expressed in HEL cells. The (+/−) and (−/−) isoforms reduced Prm1D-directed luciferase activity by 1.2-fold (p=0.0047) and 1.3-fold (p=0.0044), respectively. However, neither the exon 5(+)/KTS(+) nor exon 5(−)/KTS(+) isoforms had a significant effect on luciferase activity directed by Prm1D (p=0.2665 and p=0.9144, respectively). None of the four isoforms of WT1 had a significant effect on Prm1I-directed luciferase expression (p=0.8140, p=0.9413, p=0.8564 and p=0.8727; data not shown). Collectively, data generated from mutational analysis, EMSAs, ChIP analysis and over-expression studies indicate that −KTS isoforms of WT1 bind to elements within the Prm1 regions from −6848 to −6648 and from −6258 to −6123 and act independently to repress Prm1 activity.

### Example 7

### Effect of PMA on TPα mRNA Expression and Prm1-Directed Gene Expression in HEL Cells

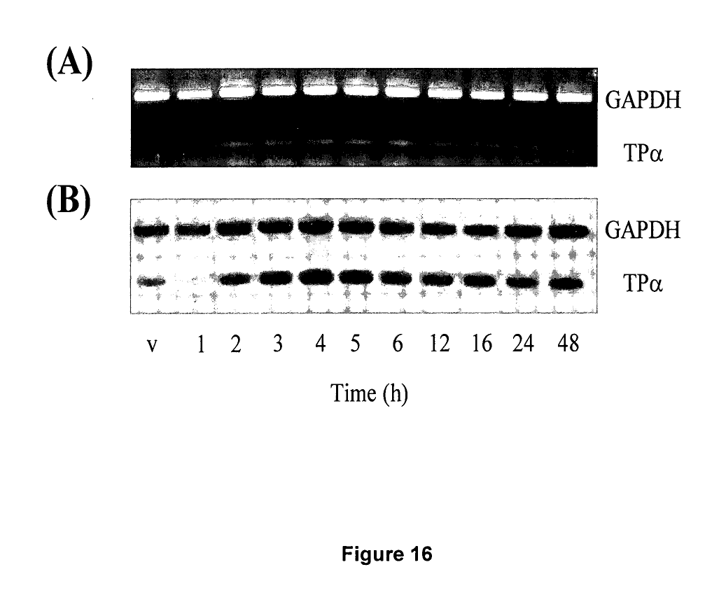

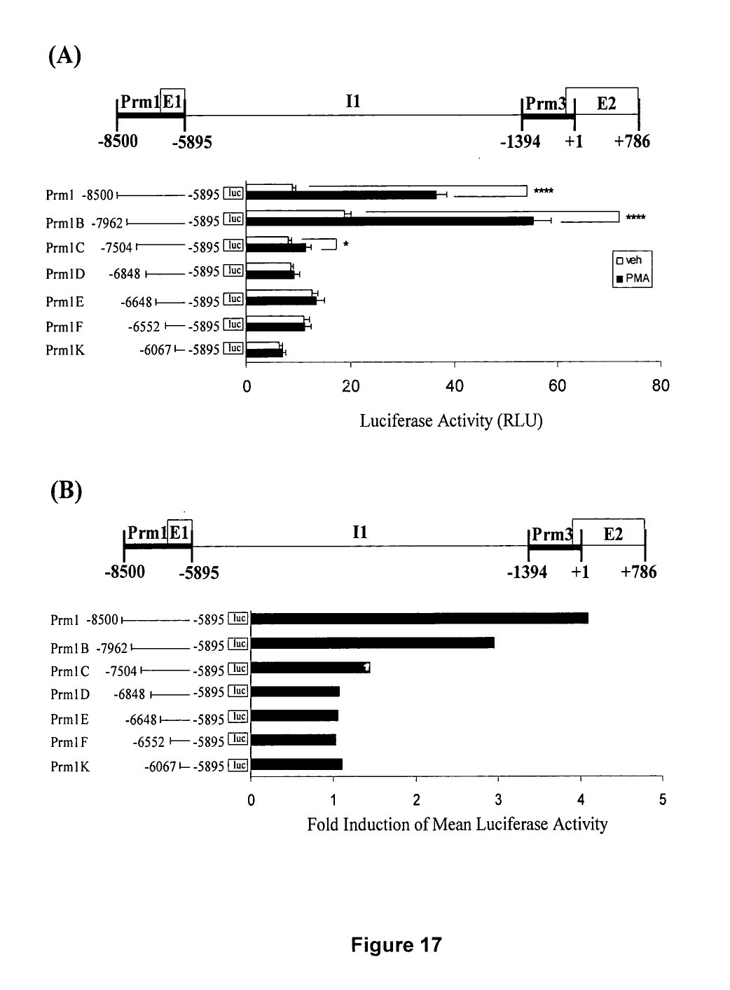

The effect of PMA-induced megakaryocytic differentiation of human erythroleukemia (HEL) 92.1.7 cells on TPα expression was investigated, as well as to identify the specific factors responsible for these changes through their regulation of Prm1. Herein, RT-PCR and Southern blot analysis (FIGS. 16A & 16B) revealed that pre-incubation of HEL cells with PMA for 2 to 48 h led to a time-dependent, sustained increase in TPα mRNA expression (FIG. 16). Moreover, genetic reporter assays established that pre-incubation of HEL cells with PMA (100 nM) for 16 h resulted in a 4-fold increase in Prm1-directed luciferase expression (p<0.0001; FIGS. 17A & 17B). In order to localize the key regulatory domains responsible for increased Prm1-directed gene expression in response to PMA, genetic reporter assays were also carried out using a series of recombinant plasmids encoding 5′ deletions of Prm1 (FIGS. 17A & 17B). Initially, and consistent with our finding that WT1 represses Prm1 activity by binding to elements within URR1 (from −8500 to −7962), 5′ deletion of Prm1 (−8500) to Prm1B (−7962) resulted in a 2.1-fold increase in basal luciferase expression in vehicle-treated HEL cells (p<0.0001; FIG. 17A). However, deletion of these nucleotides also reduced the PMA-mediated induction of Prm1-directed gene expression from 4-fold to 2.9-fold (FIGS. 17A & 17B). Thereafter, consistent with our finding that GATA-1 and Ets-1 activate Prm1 by binding to specific elements within UAR1 (from −7962 to −7717), 5′ deletion of nucleotides from Prm1B (−7962) to generate Prm1C (−7504) resulted in a 2.4-fold decrease in basal luciferase expression of Prm1 (p<0.0001; FIG. 17A). However, deletion of these nucleotides from Prm1B (−7962) to generate Prm1C (−7504) also decreased the PMA-mediated induction of Prm1-directed gene expression from 2.9-fold to 1.4-fold (FIGS. 17A & 17B). Further 5′ deletion to generate sub-fragments Prm1D (−6848), Prm1E (−6648), Prm1F (−6552) and Prm1K (−6067) abolished PMA-responsiveness of Prm1 (FIGS. 17A & 17B). Hence, these data indicate that the increased transcriptional activity of Prm1 in response to PMA is mediated mainly by cis-acting elements located between −8500 and −7504 within Prm1, while elements between −7504 and −6848 play a more minor role.

### Example 8

### Localization of the Site(s) of Action of PMA within Prm1 by Mutational Analysis

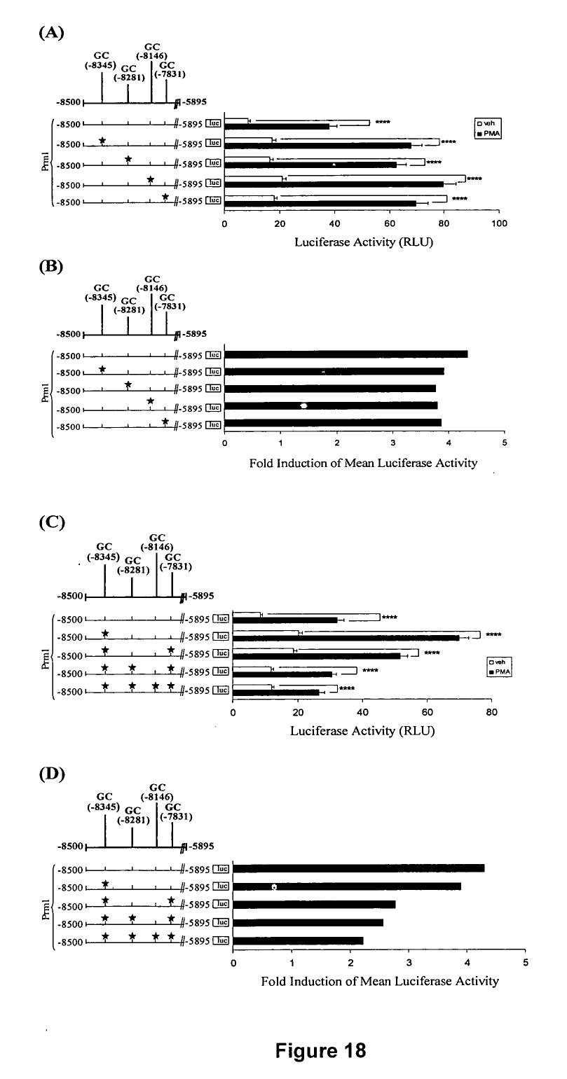

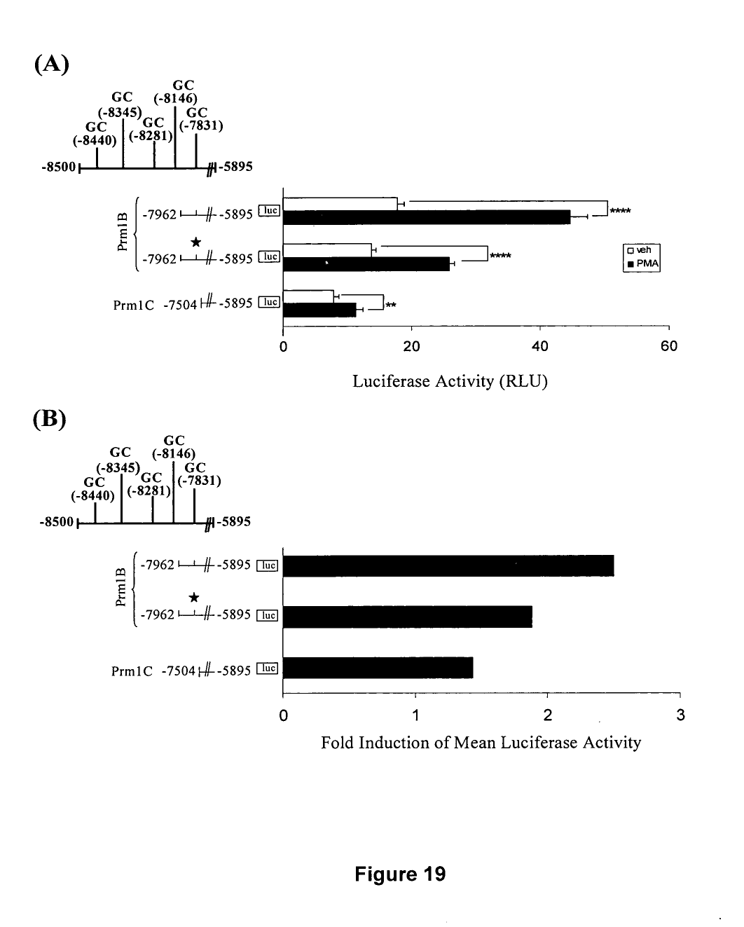

We have established that WT1 binds to GC elements at −8345, −8281 and −8146 within URR1 (from −8500 to −7962) and to another GC element at −7831 within UAR1 (from −7962 to −7717) in quiescent HEL cells to repress transcriptional activity of Prm1. Since the GC elements at −8345, −8281, −8146 and −7831 consist of overlapping binding sites for WT1/Egr1/Sp1, it was sought to determine if factors(s) binding to these elements contribute to the PMA-mediated increase in Prm1 activity. Site-directed mutagenesis of any of the individual elements at −8345, −8281, −8146 or −7831 within Prm1 did not substantially abrogate the PMA-induction of Prm1 (FIG. 18A). More specifically, the sub-fragments Prm1^{GC}*⁽⁻⁸³⁴⁵⁾, Prm1^{GC}*⁽⁻⁸²⁸¹⁾, Prm1^{GC}*⁽⁻⁸¹⁴⁶⁾ and Prm1^{GC}*⁽⁻⁷⁸³¹⁾ displayed 3.8- to 3.9-fold increases in luciferase activity in response to PMA, compared with a 4.3-fold PMA induction of Prm1 itself (FIG. 18A). To investigate whether the four GC elements act in an independent manner to contribute to the PMA-mediated increase in Prm1 activity, the effect of collectively mutating the sites within Prm1 (−8500) was examined (FIGS. 18B & 18C). The introduction of sequential mutations to generate Prm1^{GC}*^{(-8345,-8281,-8146,-7831)} from Prm1 progressively reduced the PMA-mediated induction of Prm1 activity. Specifically, PMA only yielded a 2.2-fold increase in luciferase activity directed by Prm1^{GC}*^{(-8345,-8281,-8146,-7831)} compared to the 4.3-fold increase directed by Prm1 itself (FIGS. 18B & 18C). Additionally, disruption of GC⁻⁷⁸³¹ in Prm1B (−7962), which does not contain any of the other three GC elements, resulted in an attenuation of PMA-induction of this sub-fragment from 2.5-fold to 1.9-fold (FIGS. 19A & 19B). Therefore, it is indicated that GC elements at −8345, −8281, −8146 and −7831 are responsible, at least in part, for the PMA-mediated increase in Prm1 activity in HEL cells.

The finding that mutation of any of the individual elements at −8345, −8281, −8146 and −7831 resulted in only a marginal attenuation of the PMA-mediated induction of Prm1, compared to the substantial decrease observed upon combined mutation of all four elements together to generate Prm1^{GC}*^{(-8345,-8281,-8146,-7831)}, suggests that GC elements at −8345, −8281, −8146 and −7831 act in an independent manner to contribute to the increase Prm1 activity in response to PMA.

### Example 9

### Effect of PMA on Expression of WT1, Egr1 and Sp1 Proteins in HEL Cells

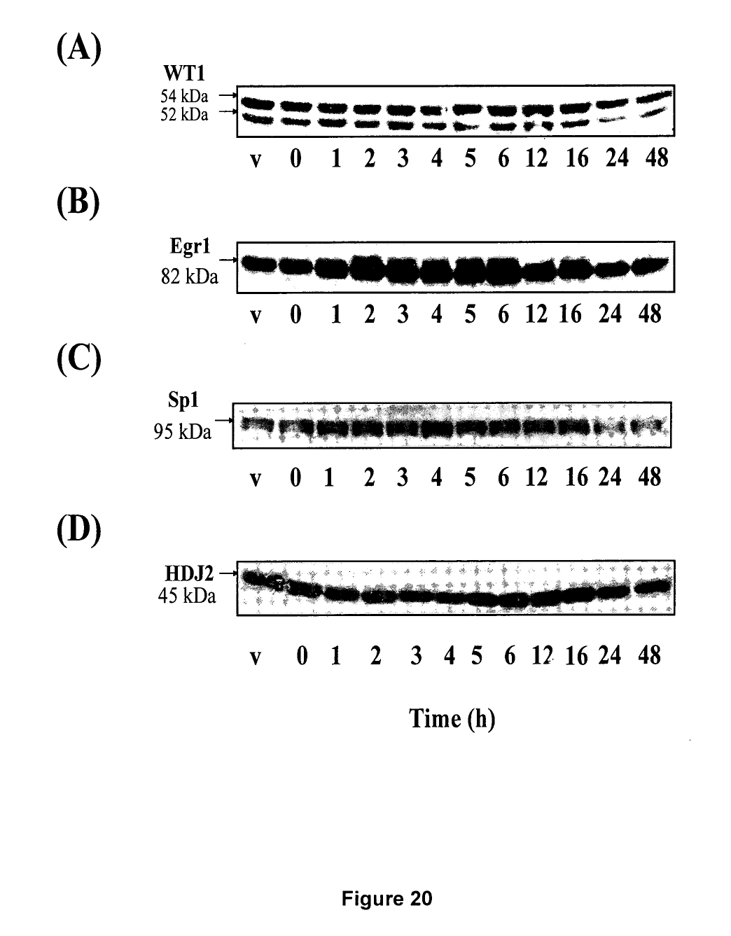

In view of the finding that PMA significantly increased Prm1-directed transcriptional activity and TPα mRNA expression in HEL cells, it was sought to identify the specific transcription factors involved. As stated, mutational analysis of GC elements representing overlapping binding sites for WT1/Egr1/Sp1 at −8345, −8281, −8146 and −7831 indicated that these elements are at least partially responsible for PMA-mediated increases in Prm1-directed luciferase expression. Therefore, it was sought to determine if the levels of expression of WT1, Egr1 or Sp1 changed upon incubation of HEL cells with PMA. While pre-incubation of HEL cells with PMA over a 48 h period did not substantially alter the expression of the WT1 doublet at 52/54 kDa (FIG. 20A), there was a slight decrease in expression of both 52 kDa and 54 kDa forms at 24 h and 48 h post-induction. There was a significant increase in Egr1 expression from 1 to 16 h, with the highest induction observed at 5 h and 6 h (FIG. 20B). At 24 h post-PMA treatment, the level of Egr1 expression in HEL cells returned to basal levels (FIG. 20B). Pre-incubation with PMA over a 48 h period did not result in appreciable changes in the expression of Sp1 (FIG. 20C), although slight increases were observed from 1 to 6 h post-stimulation. There were no appreciable changes in the expression of the molecular chaperone protein HDJ2 (DNA J homologue; FIG. 20A), which was used as an endogenous loading control, over a 48 h period post-induction compared with that of vehicle-treated cells. Collectively, these data indicate that PMA-induced differentiation of HEL cells is associated with substantial, though transient (1-16 h), increases in Egr1 expression, but does not substantially alter overall expression levels of WT1 or Sp1.

### Example 10

### Investigation of the Role of Egr1 Expression in Mediating Increased Prm1 Activity in Response to PMA

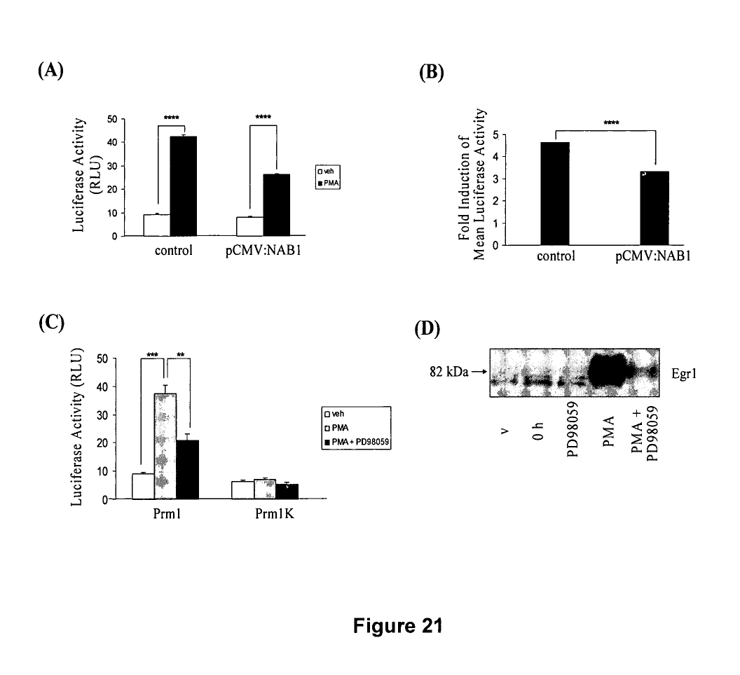

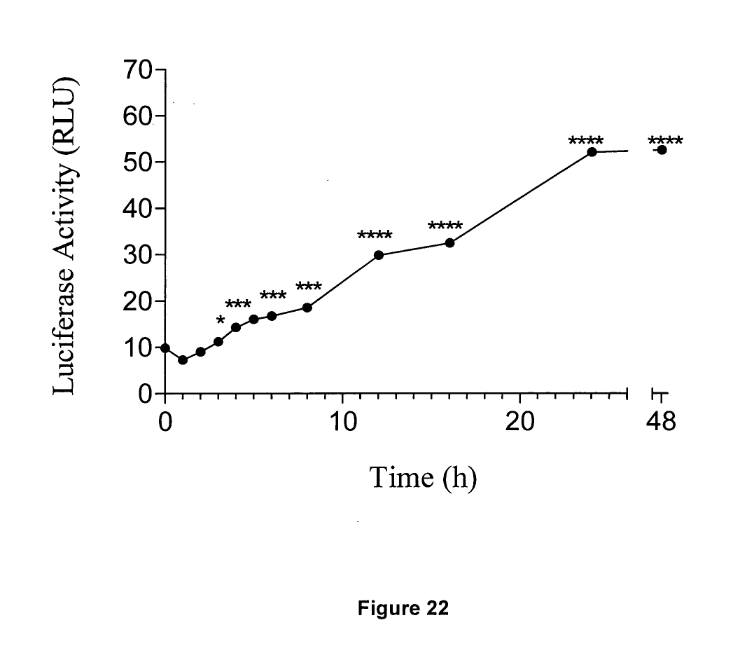

To further investigate the possible involvement of Egr1 in mediating increased Prm1-directed luciferase expression in response to PMA, the effect of ectopic expression of the constitutively expressed Egr1 co-repressor NGFI-A-binding protein 1 (NAB1) was investigated (FIG. 21A). While NAB1 over-expression did not significantly affect Prm1-directed luciferase activity in vehicle-treated HEL cells (p=0.1953), it significantly reduced the PMA-induction of Prm1 activity (p<0.0001; FIG. 21B). To investigate the role of the mitogen-activated protein kinase (MAPK) pathways in mediating the PMA-induction of Prm1 in HEL cells, the effect of the extracellular signal-regulated kinase (ERK) 1/2 inhibitor PD98059 on Prm1-directed luciferase expression was investigated (FIG. 21C). While PD98059 reduced the PMA-induction of Prm1-directed luciferase expression from 4.3-fold to 2.3-fold (p=0.0011), neither PMA alone (p=0.4721) nor PMA plus PD98059 (p=0.2693) had any significant effect on luciferase expression directed by the Prm1K sub-fragment. Furthermore, PD98059 completely abolished the PMA-induction of Egr1 protein expression in HEL cells (FIG. 21D). Collectively, these data indicate that increased expression of Egr1, mediated by PMA-induced activation of ERK signaling, is at least partly responsible for the PMA-induction of Prm1-directed luciferase activity in HEL cells. To determine the actual time required for PMA to mediate increased Prm1-directed luciferase expression, Prm1-transfected HEL cells were incubated with PMA for 0 h-48 h (FIG. 22). It was established that Prm1-directed luciferase activity was significantly increased within 4 h of incubation with PMA (p=0.029; FIG. 22) and was continuously increased for the duration of the 48 h incubation (p<0.0001). From the time course assay, it appeared that PMA-induction of Prm1-transcriptional activity was multi-phasic, with initial activity plateauing at 5-8 h post-induction and a subsequent phase plateauing at ˜12-16 h followed by a more sustained activity at 24-48 h post-treatment.

### Example 11

### In Vivo Binding of WT1, Egr1 and Sp1 to GC Elements Between −8460 and −8006 within Prm1

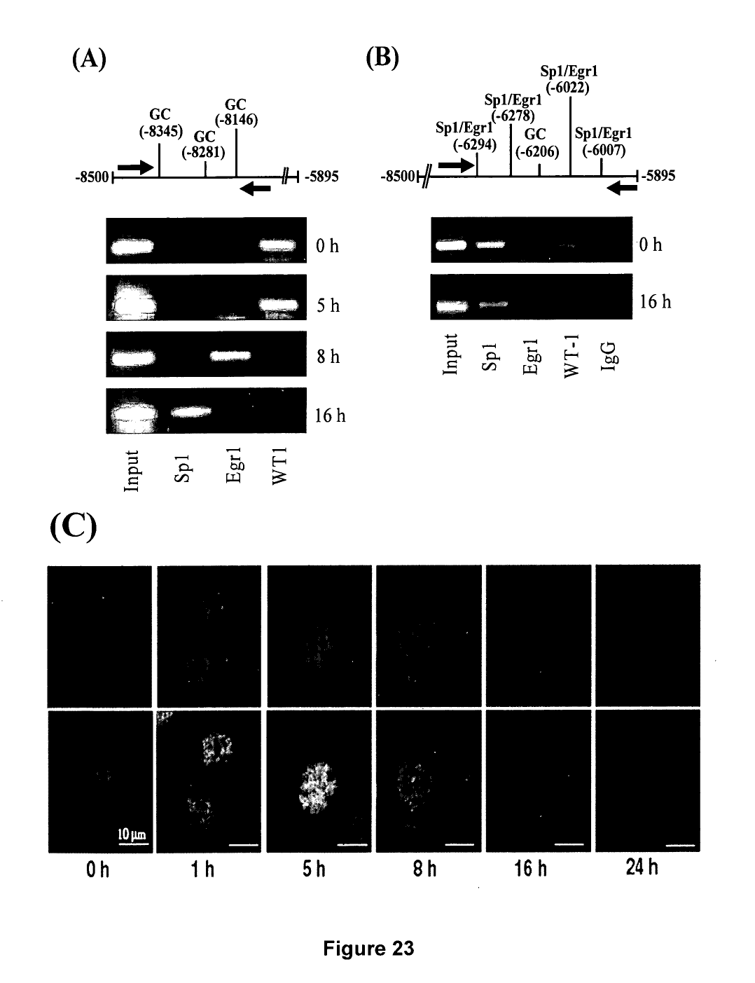

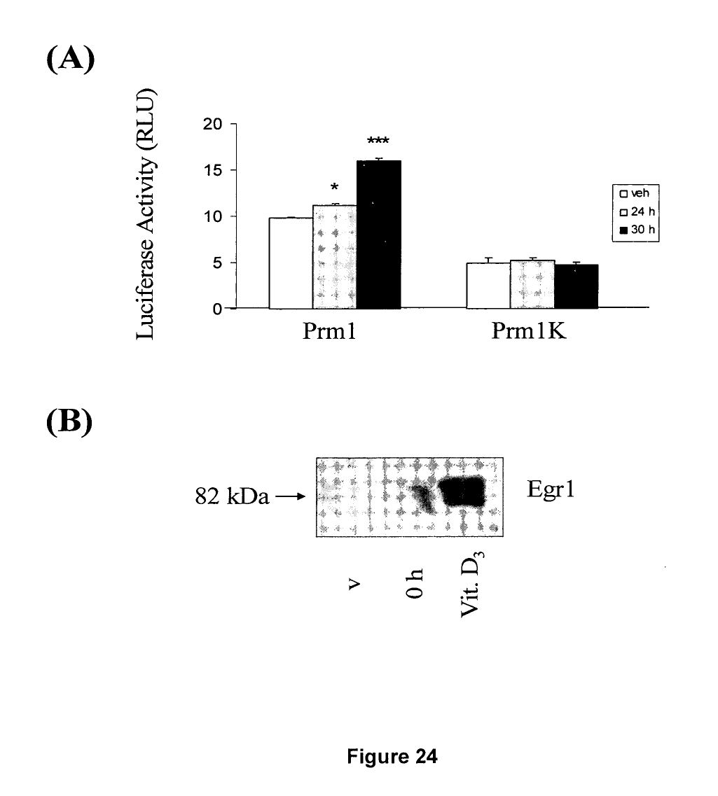

To investigate the molecular identity of transcription factor(s) actually regulating Prm1 activity in response to PMA treatment, chromatin immunoprecipitation (ChIP) assays were carried out using antibodies directed to endogenous WT1, Egr1 and Sp1 and chromatin extracted from HEL cells pre-incubated with PMA for 5, 8 or 16 or 24 h, where non-treated or vehicle-treated HEL cells served as controls (FIGS. 24A & B). Initially, PCR analyses of ChIPs were carried out using primers to amplify the distal 5′ Prm1 region located between −8460 and −8006, containing GC elements at −8345, −8281 and −8146 (FIG. 24A). In non-treated (0 h) or in vehicle-treated (data not shown) HEL cells, PCR amplification yielded products from DNA recovered from both the input chromatin and from an anti-WT1 immunoprecipitate, but not from anti-Sp1 or anti-Egr1 immunoprecipitates (FIG. 24A). PCR analysis using ChIP samples generated from HEL cells pre-incubated with PMA for 5 h resulted in amplification of DNA recovered from both the input chromatin and from an anti-WT1 immunoprecipitate, and to a lesser extent from an anti-Egr1 immunoprecipitate. However, no amplicon was generated from an anti-Sp1 immunoprecipitate (FIG. 24A; 5 h). Conversely, following pre-incubation with PMA for 8 h, PCR resulted in amplification of DNA recovered from both the input chromatin and from an anti-Egr1 immunoprecipitate, but not from anti-WT1 or anti-Sp1 immunoprecipitates (FIG. 23A; 8 h). ChIP analysis using HEL cells pre-incubated with PMA for 16 h (FIG. 23A; 16 h) or 24 h (data not shown) yielded amplicons from the input chromatin and an anti-Sp1 immunoprecipitate, but not from anti-WT1 or anti-Egr1 immunoprecipitates. Hence, it appears that following PMA-induced differentiation, there are distinct, multi-phasic patterns of binding of WT1, Egr1 and Sp1 to the GC elements located within the −8460 to −8006 region of Prm1 chromatin in HEL cells that may account for the observed time-dependent induction in TPα mRNA expression (FIG. 16) and Prm1-directed luciferase expression (FIG. 22). It has previously been demonstrated that Sp1, Egr1 and WT1 bind to GC elements in the proximal “core” Prm1 located between −6320 and −5895. Hence, ChIP analysis of this region was carried out to investigate if changes in the pattern of Sp1, Egr1 or WT1 binding occurred upon PMA-induced differentiation of HEL cells. Consistent with the finding that PMA did not lead to an induction of luciferase expression directed by Prm1E, Prm1F and Prm1K sub-fragments consisting of “core” Prm1 sequences (FIGS. 16A & 16B), ChIP analysis revealed similar binding patterns for WT1, Egr1 and Sp1 in non-treated quiescent cells (0 h) and in cells treated with PMA for 5, 8 or 16 (FIG. 23B and data not shown). Collectively, these data indicate that while PMA-treatment of HEL cells does not lead to significant changes in the relative levels of WT1, Egr1 and Sp1 binding to the proximal “core” Prm1, it leads to substantial changes in the pattern of binding of these factors to distal upstream GC-enriched elements. Specifically, in non-treated quiescent HEL cells, WT1 binds to the 5′ Prm1 region from −8460 to −8006. However, following exposure of cells to PMA for 5 h, and coincident with its increased expression (FIG. 20), Egr1 appears to bind to this region in vivo, albeit to a much lesser extent than WT1. Following pre-incubation of HEL cells with PMA for 8 h, a substantial increase in Egr1 binding and an associated decrease in WT1 binding was observed. Conversely, in HEL cells pre-incubated with PMA for 16 h, Sp1 is the predominant protein bound. Therefore, it is suggested that these distinct patterns of binding of WT1, Egr1 and Sp1 (FIGS. 23A & 23B) are accountable for initial and sustained increases in Prm1-directed luciferase expression in response to PMA-induced differentiation of HEL cells (FIG. 22). Thereafter, the intracellular localization of WT1 in non-treated and PMA-stimulated HEL cells was investigated. In the absence of (FIG. 23C; 0 h) and 1 h post-PMA treatment (FIG. 23C; 1 h), WT1 was almost exclusively localised to the nucleus while at 5 h (FIG. 23C; 5 h) and, in particular at 8 h (FIG. 23C; 8 h), there was a redistribution of WT1 to the cytosolic fraction. At 16 and 24 h (FIG. 23C; 16 h & 24 h), a substantially higher proportion of WT1 was located in the cytosol than in the nucleus. Cytosolic WT1 appeared to be associated with punctate vesicular structures.

### Example 12

### Effect of 1α, 25-Dihydroxy-Vitamin D₃ on Prm1-Directed Luciferase Expression and Egr1 Protein Expression in HEL Cells

Thereafter, it was sought to investigate the effect of PMA-independent megakaryocytic differentiation of HEL cells on Prm1-directed gene expression. Genetic reporter assays established that pre-incubation of HEL cells with 1α, 25-dihydroxy-vitamin D₃ (Vitamin D₃) for 30 h increased Prm1-directed luciferase expression by 1.6-fold (FIG. 24A; p=0.0002). Conversely, pre-incubation of HEL cells with Vitamin D₃ for 24 h or 30 h had no significant effect on luciferase expression directed by Prm1K (p=0.8037 and p=0.8612, respectively). Furthermore, Vitamin D₃ led to a substantial increase in Egr1 protein expression in HEL cells (FIG. 24B). Collectively, these data indicate that Vitamin D₃-induced differentiation of HEL cells leads to increased expression of Egr1, as well as induction of Prm1-directed luciferase activity in HEL cells.

### Example 13

### Modified Prm1 Promoter-Directed Gene Expression in HEL (Human Erythroleukemia) 92.1.6, EA.hy 926 (Human Endothelial), HEK (Human Embryonic Kidney) 293, 1° hAoSMC (Primary Human Aortic Smooth Muscle), and WI-38 (Human Lung Fibroblast) Cells

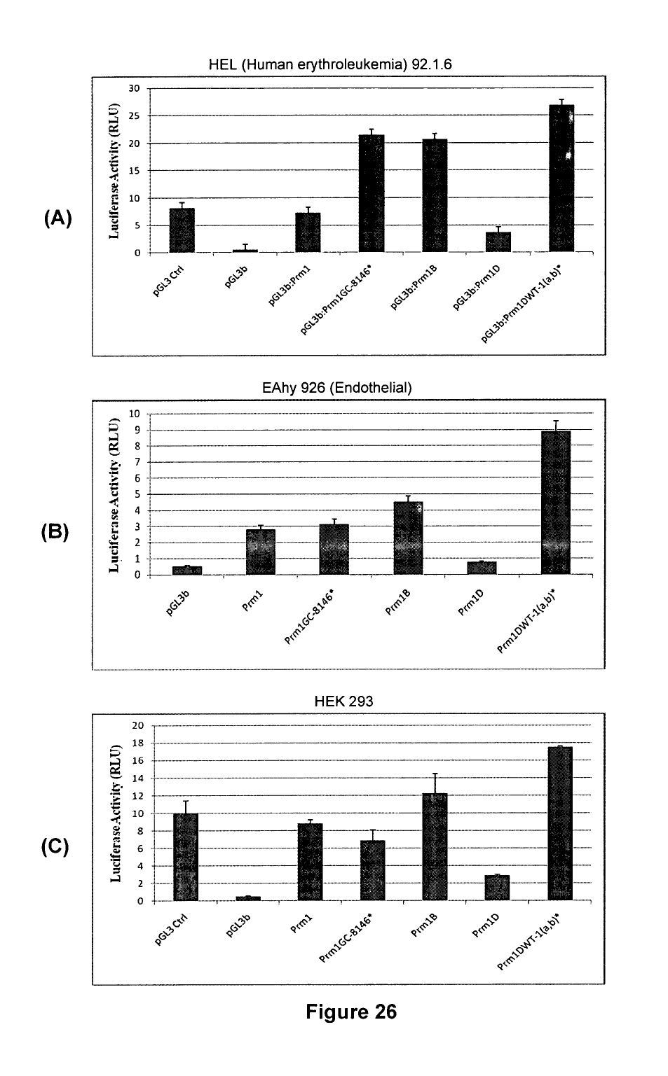

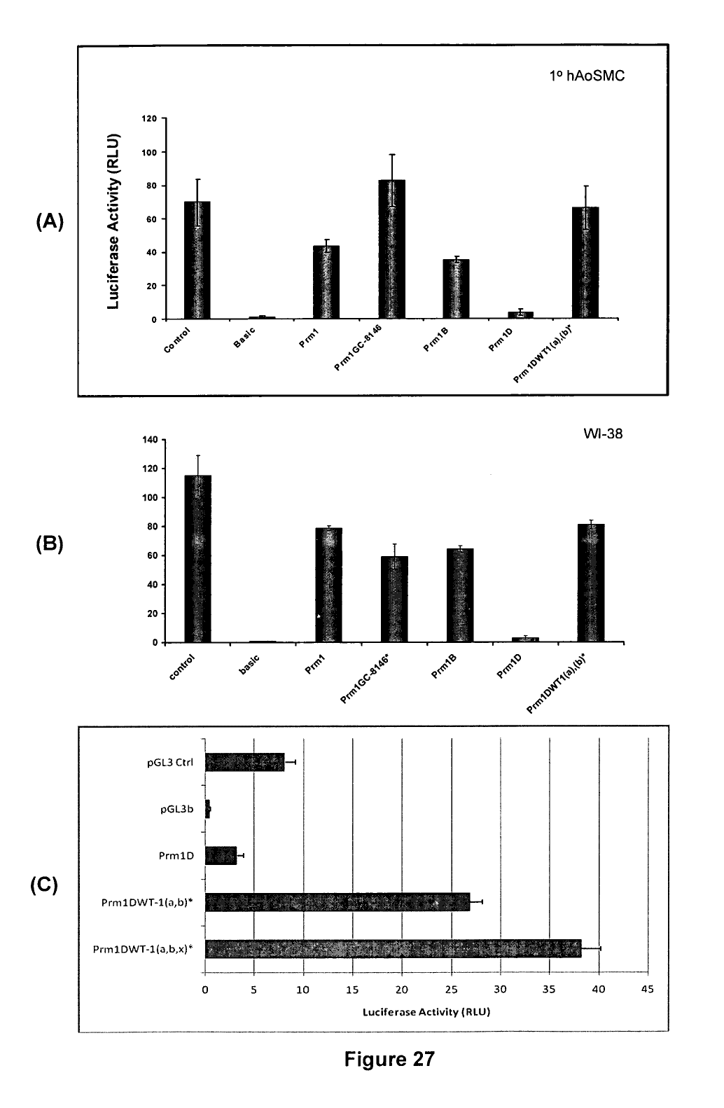

The following plasmids, encoding 5′ deletion fragments of Prm1, pGL3B:Prm1, pGL3B:Prm1^{GC-8146}*, pGL3B:Prm1B, pGL3B:Prm1D, pGL3B: Prm1D^{WT1(a),(b)}* and, as controls, pGL3Control (Promega) & pGL3Basic (Promega; empty vector) were co-transfected with pRL-TK, in the case of the luciferase assays (transfected without pRL-TK in the case of Western Blotting analysis), into the following cell lines;
1) HEL (Human erythroleukemia) 92.1.6 (FIG. 26A);
2) EA.hy 926 (human endothelial) (FIG. 26B);
3) HEK (Human embryonic kidney) 293 (FIG. 26C);
4) 1° hAoSMC (primary human aortic smooth muscle cell) (FIG. 27A); and
5) WI-38 (human lung fibroblast) (FIG. 27B).

Their relative promoter-directed luciferase activity was determined. The pGL3-Control Vector contains the firefly luciferase gene under the control of the SV40 promoter and enhancer sequences, resulting in strong expression of luciferase in many types of mammalian cells. This plasmid is useful in monitoring transfection efficiency, in general, and is a convenient internal standard for promoter and enhancer activities expressed by pGL3 recombinants.

TABLE 3A
HEL (Human erythroleukemia) 92.1.6 cells

| DNA | Mean | SEM |
|---|---|---|
| pGL3b | 0.506 | 0.055 |
| pGL3 ctrl | 8.13 | 1.068 |
| pGL3b:Prm1 | 7.28 | 0.22 |
| pGL3b:Prm1^{GC-8146}* | 21.5 | 0.525 |
| pGL3b:Prm1B | 20.68 | 0.17 |
| pGL3b:Prm1D | 3.7 | 0.326 |
| pGL3b:Prm1D^{WT-1(a, b)}* | 26.87 | 1.29 |

| Fragment 1 | Fragment 2 | Statistical significance |
|---|---|---|
| Prm1 vs | Prm1^{GC-8146}* | *** |
| Prm1 vs | Prm1B | *** |
| Prm1D vs | Prm1D^{WT-1(a, b)}* | *** |

TABLE 3B
EAhy 926 (Endothelial) cells

| DNA | Mean | SEM |
|---|---|---|
| pGL3b | 0.51 | 0.06 |
| pGL3 ctrl | 49.97 | 0.612 |
| pGL3b:Prm1 | 2.81 | 0.25 |
| pGL3b:Prm1^{GC-8146}* | 3.11 | 0.327 |
| pGL3b:Prm1B | 4.52 | 0.332 |
| pGL3b:Prm1D | 0.755 | 0.06 |
| pGL3b:Prm1D^{WT-1(a, b)}* | 8.89 | 0.612 |

| Fragment 1 | Fragment 2 | Statistical significance |
|---|---|---|
| Prm1 vs | Prm1^{GC-8146}* | ns |
| Prm1 vs | Prm1B | ** |
| Prm1D vs | Prm1D^{WT-1(a, b)}* | *** |

TABLE 3C
HEK 293 cells

| DNA | Mean | SEM |
|---|---|---|
| pGL3b | 0.51 | 0.06 |
| pGL3 ctrl | 9.97 | 1.4 |
| pGL3b:Prm1 | 8.83 | 0.395 |
| pGL3b:Prm1^{GC-8146}* | 6.9 | 1.19 |
| pGL3b:Prm1B | 12.25 | 2.23 |
| pGL3b:Prm1D | 2.89 | 0.145 |
| pGL3b:Prm1D^{WT-1(a, b)}* | 17.51 | 0.098 |

| Fragment 1 | Fragment 2 | Statistical significance |
|---|---|---|
| Prm1 vs | Prm1^{GC-8146}* | ns |
| Prm1 vs | Prm1B | * |
| Prm1D vs | Prm1D^{WT-1(a, b)}* | *** |

TABLE 3D
1° hAoSMC cells

| DNA | Mean | SEM |
|---|---|---|
| pGL3Control | 69.861 | 14.086 |
| pGL3Basic | 1.086 | 0.561 |
| pGL3BPrm1 | 43.733 | 4.043 |
| pGL3BPrm1GC-8146 | 82.668 | 15.400 |
| pGL3BPrm1B | 35.300 | 1.842 |
| pGL3BPrm1D | 3.611 | 1.901 |
| pGL3BPrm1DWT1(a), (b)* | 66.194 | 12.755 |

| Fragment 1 | Fragment 2 | Statistical significance |
|---|---|---|
| Prm1 vs | Prm1^{GC-8146}* | ** |
| Prm1 vs | Prm1B | ns |
| Prm1D vs | Prm1D^{WT-1(a, b)}* | *** |

TABLE 3E
WI-38 cells

| DNA | Mean | SEM |
|---|---|---|
| pGL3Control | 115.09 | 13.9 |
| pGL3Basic | 0.82 | 0.09 |
| pGL3BPrm1 | 78.68 | 1.58 |
| pGL3BPrm1GC-8146 | 59.01 | 8.67 |
| pGL3BPrm1B | 64.33 | 2.11 |
| pGL3BPrm1D | 2.75 | 1.50 |
| pGL3BPrm1DWT1(a), (b)* | 80.80 | 3.08 |

| Fragment 1 | Fragment 2 | Statistical significance |
|---|---|---|
| Prm1 vs | Prm1^{GC-8146}* | ns |
| Prm1 vs | Prm1B | ns |
| Prm1D vs | Prm1D^{WT-1(a, b)}* | *** |

### Example 14

### Modified Prm1D Derivatives (Prm1D, Prm1D^{WT1(a)}*^{(b)}*, and Prm1D^{WT1(a)}*^{(b)}*^{(X)}*) to Direct Gene Expression in HEL Cells

The Prm1D^{WT1(a)}*^{(b)}*^{(X)}* promoter was generated as described herein above, and comprises the nucleic acid sequence defined in SEQ ID NO:124). In brief, quickchange site-directed mutagenesis was performed at each of the above-mentioned nucleotide positions (from G C, which knocks out the activity of the WT-1^{(x)} site (@−6800); (from CC→AT), which knocks out the activity of the WT-1^{(a)} site (@−6717); and (from CC→TA), which knocks out the activity of the WT-1^{(b)} site (@−6206); all as confirmed by MatInspector™ analysis. The identity of the Prm1D-derived promoter, Prm1D^{WT1(a)}*^{(b)}*^{(X)}*,comprising the mutations at each of nucleic acid positions −6717, −6206, and −6800, was verified through DNA sequencing (See SEQ ID NO:124). A multiple sequence alignment (not shown) confirmed that Prm1DWT1^{(a)}*^{(b)}*^{(X)}*, had the three base changes (WT-1^{(X)}* @ −6800, WT-1^{(a)}* @ −6717 and WT-1^{(b)}* @−6206 respectively). Investigation of the Prm1D derivatives (Prm1D, Prm1D^{WT1(a)}*^{(b)}*, and Prm1D^{WT1(a)}*^{(b)}*^{(X)}* to direct luciferase expression in HEL cells, where WT1^{(a)}* refers to the mutation of the WT1 site at −6717, WT1^{(b)}* refers to the mutation of the WT1 site at −6206 and WT1^{(X)}* refers to the mutation of a repressor site at −6800 (FIG. 27C).

TABLE 4

| DNA | Mean | SEM |
|---|---|---|
| pGL3b | 0.506 | 0.055 |
| pGL3 ctrl | 8.13 | 1.068 |
| pGL3b:Prm1D | 3.7 | 0.326 |
| pGL3b:Prm1D^{WT-1(a, b)}* | 26.87 | 1.29 |
| pGL3b:Prm1D^{WT-1(a, b, x)}* | 38.26 | 1.9 |

| Fragment 1 | Fragment 2 | Statistical significance |
|---|---|---|
| Prm1D vs | Prm1D^{WT-1(a, b)}* | *** |
| Prm1D vs | Prm1D^{WT-1(a, b, x)}* | *** |
| Prm1D^{WT-1(a, b)}* | Prm1D^{WT-1(a, b, x)}* | *** |

### Example 15

### Analysis of Promoter Strength as Measured by Relative Transcriptional Activity

The ability of the native (unmodified) Prm1 promoter, and each of the Prm1 derivatives (Prm1D, Prm1D^{WT-1(a,b)}*, Prm1^{GC-8146)}*, Prm1B, and Prm1D^{WT-1(a,b,x)}*) to drive protein expression in a panel of various cell types was evaluated, and compared to the ability of the pGL3Control vector. As previously described, the pGL3Control vector has the same luciferase gene under the control of an SV40 promoter and enhancer sequences, and results in strong expression of luciferase in different types of mammalian cell types. The ratio of luciferase expression by Prm1 (or its derivatives) relative to that directed by the pGL3Control plasmid (where the luciferase gene is under the control of the SV40 promoter and enhancer sequences) was determined to establish the relative strengths of the promoters in the different cell types.

TABLE 5A
Strength of Prm1 promoter

| | pGL3Control/ | pGL3B:Prm1/ | |
|---|---|---|---|
| Cell Line | RLU | RLU | Ratio |
| HEL | 8.13 | 7.28 | 0.90 |
| EA.hy 926 | 49.97 | 2.81 | 0.06 |
| HEK | 9.97 | 8.83 | 0.89 |
| 1° hAoSMC | 69.86 | 43.73 | 0.63 |
| WI-38 | 115.09 | 78.68 | 0.68 |

TABLE 5B
Strength of Prm1^{WT1(a, b)}* promoter

| | | pGL3B: | |
|---|---|---|---|
| | pGL3Control/ | Prm1^{WT1(a, b)}*/ | |
| Cell Line | RLU | RLU | Ratio |
| HEL | 8.13 | 26.89 | 3.31 |
| EA.hy 926 | 49.97 | 8.89 | 0.178 |
| HEK | 9.97 | 17.51 | 1.76 |
| 1° hAoSMC | 69.86 | 66.19 | 0.95 |
| WI-38 | 115.09 | 80.80 | 0.79 |

TABLE 5C
Strength of Prm1^{GC-8146}* promoter

| | | pGL3B:Prm | |
|---|---|---|---|
| | pGL3Control/ | Prm1^{GC-8146}*/ | |
| Cell Line | RLU | RLU | Ratio |
| HEL | 8.13 | 21.5 | 2.65 |
| EA.hy 926 | 49.97 | 3.11 | 0.06 |
| HEK | 9.97 | 6.9 | 0.69 |
| 1° hAoSMC | 69.86 | 82.67 | 1.18 |
| WI-38 | 115.09 | 59.01 | 0.51 |

TABLE 5D
Strength of Prm1B promoter

| | pGL3Control/ | pGL3B:Prm1B/ | |
|---|---|---|---|
| Cell Line | RLU | RLU | Ratio |
| HEL | 8.13 | 20.68 | 2.54 |
| EA.hy 926 | 49.97 | 4.52 | 0.09 |
| HEK | 9.97 | 12.25 | 1.23 |
| 1° hAoSMC | 69.86 | 35.3 | 0.51 |
| WI-38 | 115.09 | 64.33 | 0.56 |

TABLE 5E
Strength of Prm1D promoter

| | pGL3Control/ | pGL3B:Prm1D/ | |
|---|---|---|---|
| Cell Line | RLU | RLU | Ratio |
| HEL | 8.13 | 3.7 | 0.455 |
| EA.hy 926 | 49.97 | 0.755 | 0.015 |
| HEK | 9.97 | 2.89 | 0.29 |
| 1° hAoSMC | 69.86 | 3.6 | 0.05 |
| WI-38 | 115.09 | 2.75 | 0.024 |

It can be seen that the Prm1 promoter is particularly strong in HEL cells and in HEK (Kidney) cells, relative to the SV40 Promoter and enhancer; is comparible in AoSMCs; slightly weaker in the WI-38 (lung fibroblasts) and weak in endothelial cells. Prm1 is comparible to the SV40 Promoter and enhancer in directing luciferase activity in many of the cell types examined. However, these data also demonstrate that Prm1WT1(a,b)* provides a stronger promoter activity when compared to Prm1 in all cell lines. Moreover, the modified promoters of the present invention, unlike the SV40 promoter (which needs an enhancer element to efficiently drive transcription), drives expression without the requirement of specific enhancer sequences.

### Example 16

### Modified Prm1-Directed Human Tissue Factor Protein Expression in HEL Cells

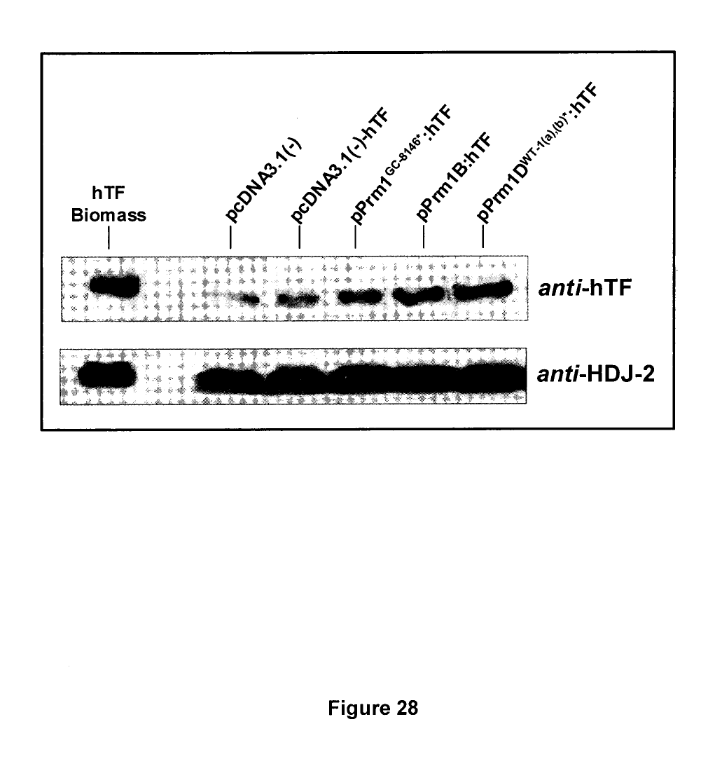

The plasmid pcDNA3.1(−):hTF encodes human tissue factor (hTF) under the control of the widely used strong cytomegalovirus (CMV) promoter, and was generated as described herein above. The nucleic acid sequence of the recombinant plasmid was confirmed by DNA sequence analysis. A multiple sequence alignment (not shown) confirmed that the resultant forward primer product, as defined by SEQ ID NO:125, was 100% identical with nucleotides 169-1000 of the known nucleic acid sequence of the human tissue factor gene (Genbank Accession No BC011029). A multiple sequence alignment (not shown) confirmed that the reverse complementary sequence, as defined by SEQ ID NO:128, of the reverse primer product, as defined by SEQ ID NO:127, was 100% identical with nucleotides 169-1056 of the known nucleic acid sequence of the human tissue factor gene (Genbank Accession No BC011029). Moreover, in silico translation of SEQ ID NO:125 generated an amino acid sequence as defined by SEQ ID NO: 126, and in silico translation of SEQ ID NO:128 generated an amino acid sequence as defined by SEQ ID NO: 129. A multiple sequence alignment (not shown) confirmed that SEQ ID NO:126 was 100% identical to SEQ ID NO:129. Taken together, these data confirm that there are no point mutations and the DNA to protein translation is correct, as are the cloning sites and vector sequences. To confirm these data, HEL cells were co-transfected with 2 μg pcDNA3.1(−), pcDNA3.1(−)-hTF, pPrm1^{GC,-8146}*:hTF, pPrm1B:hTF and pPrm1D^{WT1(a),(b)}* along with 200 ng pRL-TK. Cells were analysed 48 h post-transcfection by western blotting (60 μg of whole cell protein per lane) along with a hTF Biomass control using anti-hTF (upper panel) antibody; to confirm equal loading, we stripped and rescreen blots with an anti-HDJ-2 antibody (lower panel) (FIG. 28). Additionally, the three Prm1 derivatives tested, namely, Prm1 GC-8146*, Prm1B and Prm1D WT-1 (a,b)*, drive human tissue factor expression in megakaryocytic HEL cells at a level greater when compared to the control vector used, namely, pcDNA3.1(−):hTF. The plasmid pcDNA3.1(−):hTF encodes human tissue factor (hTF) under the control of the widely used strong cytomegalovirus (CMV) promoter. It can be seen that the level of human tissue factor expression by promoters derived from Prm1 (for example, Prm1 GC-8146*, Prm1B and Prm1D WT-1 (a,b)*) was significantly greater than the level expressed in the control transfected cells (in the presence of pcDNA3.1(−)) or by that directed by the CMV promoter (in the presence of pcDNA3.1(−):hTF), indicating that the promoters of the present invention provide an unexpected technical advantage compared to promoters currently available in the state of the art.

### DISCUSSION

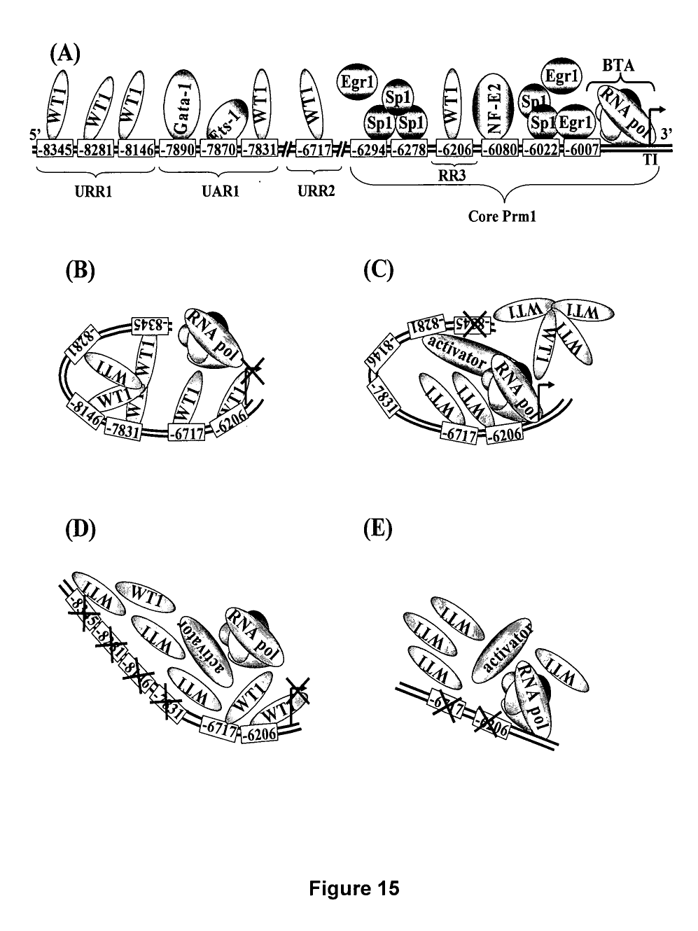

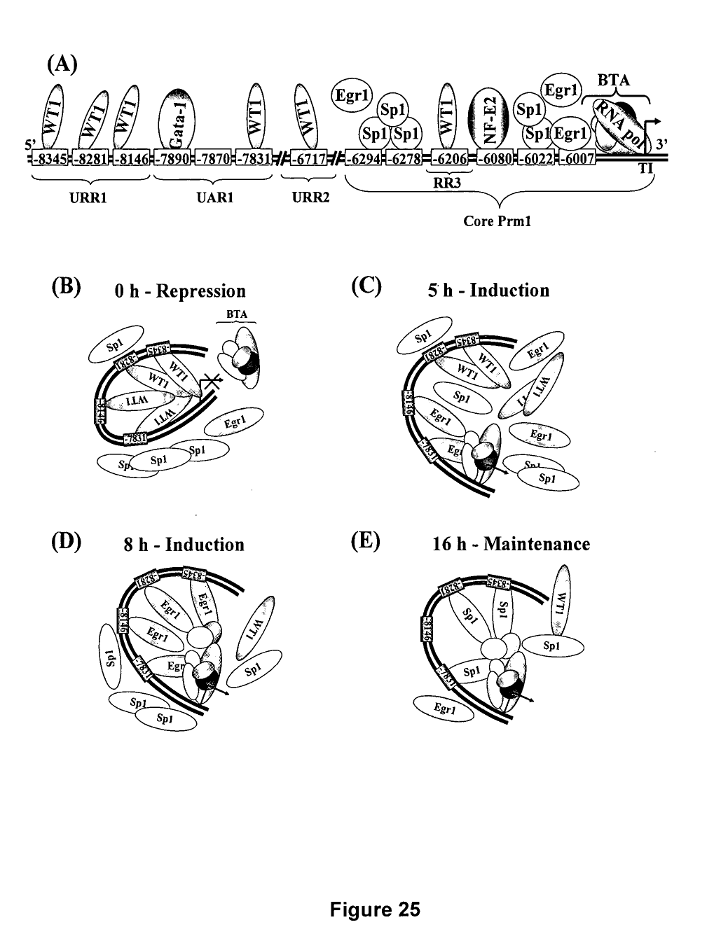

In humans, TXA₂ signals through the TPα and TPβ isoforms of its cognate G-protein coupled receptor. Imbalances in the levels of TXA₂ and TP are implicated in a range of cardiovascular disorders, but the relative extent to which TPα and TPβ contribute to such pathologies is unknown. As TPα and TPβ are under the transcriptional control of distinct promoters, identification of the factors regulating Prm1 and Prm3 may lead to a greater understanding of their contributory roles in health and disease. Through studies aimed at characterizing Prm3, AP1 and Oct-2 were identified as the key factors regulating its basal activity in HEL cells. Moreover, the endogenous cyclopentone 15-deoxy-Δ12,14-prostaglandin J₂ (15d-PGJ₂), a peroxisome proliferator-activated receptor (PPAR)γ ligand, suppressed the transcriptional activity of Prm3 but had no effect on Prm1. Additionally, the synthetic thiazolidinedione (TZD) PPARγ ligands rosiglitazone and troglitazone, used in the treatment of type II diabetes mellitus, selectively suppressed Prm3 activity, without affecting Prm1. An implication from those studies is that the TZD PPARγ ligands may have combined therapeutic benefits in the treatment of type II diabetes and of the associated cardiovascular disease, partly due to their suppression of TPβ expression. Prm1 represents the main promoter within the human TP gene, but despite this, to date the factors regulating its expression such as within the vasculature or indeed other tissue/cell types remain largely undefined. Herein, we sought to identify the key factors regulating basal Prm1 activity in the HEL 92.1.7 megakaryocytic cell line. Prm1 belongs to the class of promoters that lack TATA or CAAT elements. Many TATA-less promoters contain multiple GC-rich elements in their proximal promoter from which transcription can be activated by the ubiquitously expressed zinc finger transcription factor Sp1 by its recruitment of multi-subunit complex(es) involving TF_{II}D. Adjacent Sp1 sites may activate transcription independently from one another, or synergistically through formation of homomultimeric complexes. Early growth response protein (Egr)1, another zinc finger transcription factor, also has a GC-rich binding site and because of the similarity in their consensus elements, adjacent or overlapping sites for Sp1 and Egr1 are frequently found in promoter sequences. By mutational analysis and EMSAs, four functional overlapping Sp1/Egr1 elements were identified within the proximal region of Prm1. EMSA and supershift analyses indicated a role for Sp1 and Egr1 binding to each of these elements in vitro, while ChIP analysis confirmed the in vivo binding of both endogenous Sp1 and endogenous Egr1 to the proximal Prm1 region of chromatin extracted from HEL cells. Several studies have shown that where overlapping Sp1/Egr1 sites occur in proximal promoter regions, Egr1 negatively regulates Sp1-mediated basal transcription by competitively binding to the overlapping element. The four functional Sp1/Egr1 elements identified herein within the proximal Prm1 were adjacent overlapping sites that, through mutational studies, were shown to cooperatively regulate Prm1. Consistent with this, herein, EMSAs confirm that Sp1 and Egr1 generally compete for the same sites within Prm1. Moreover, over-expression of recombinant Egr1 in HEL cells led to a modest, but significant, reduction in the level of luciferase expression directed by the proximal Prm1. It is likely that over-expression of Egr1 may have led to a more pronounced reduction in Prm1-directed gene expression if the total amount of transfected DNA herein was not limited by the luciferase-based reporter assay itself. Therefore, the combined assessment of these studies suggest that it is likely that Sp1 activates transcription from the TATA-less Prm1 of the TP gene, and Egr1 negatively regulates this transcription by competing with Sp1 for binding at each of the four overlapping Sp1/Egr1 sites. In addition to the Sp1/Egr1 sites, we also identified a functional NF-E2/AP1 element by mutational analysis of the proximal Prm1. NF-E2 is a heterodimeric transcription factor that binds to the consensus sequence (T/C)GCTGA(G/C)TCA(T/C), with a core AP1 motif (in italics). While data from EMSA, supershift and ChIP assays confirmed that endogenous NF-E2 specifically bound to the NF-E2/AP1 element within Prm1 both in vitro and to chromatin in vivo in HEL cells, further studies are necessary to comprehensively investigate the possible binding of AP1 components such as Jun B, Jun D, c-Fos, FosB, Fra1 and Fra2, to the proximal Prm1. The heterodimeric NF-E2 is composed of a tissue-restricted p45 subunit associated with a ubiquitously-expressed p18 member of the Maf family. Expression of p45 is restricted mainly to haematopoietic progenitors, as well as differentiated erythroid and megakaryocytic cells, mast cells and granulocytes. Originally it was thought that the primary role of NF-E2 was in erythroid development due to its regulation of the porphobilinogen deaminase and globin genes. However, p45-deficient mice exhibited only mild disruption to erythropoiesis but displayed severe thrombocytopenia (<5% of normal platelet count) and high mortality due to haemorrhage. Notable amongst its megakaryocytic targets, NF-E2 regulates expression of the human and rodent thromboxane synthases, platelet-specific Rab27b, β1-tubulin and caspase-12. Thus, it is suggested that NF-E2 acts as a critical mediator of platelet shedding, regulating a subset of genes involved in late-stage megakaryocyte maturation. Consistent with this, herein, we report that Prm1 of the TP gene is also a bone fide target of NF-E2, suggesting that it plays a critical role in regulating expression of TPα during megakaryocytic differentiation and in platelets in humans. Collectively, whilst our data have established a role for Sp1, Egr1 and NF-E2 in regulating the core proximal Prm1, they do not exclude the possible involvement of other regulatory elements/factors in this region. Most eukaryotic promoters contain UAS and URS. Herein, 5′ deletion analyses revealed two UAS and two URS regions within Prm1. Mutational analysis of the first UAS region (−7962 to −7717) identified functional GATA and Ets elements capable of regulating Prm1 and the heterologous SV40 promoter in HEL cells. EMSAs confirmed the presence of nuclear factors in HEL cells capable of binding to the GATA and Ets elements in vitro. Due to the complex binding patterns of the probe encoding the GATA and Ets elements, supershift assays failed to provide a clear interpretation of the identities of specific transcription factors that bind to these sites in vitro. However, supershift assays were superseded by in vivo ChIP analysis, which confirmed binding of endogenous GATA-1 and endogenous Ets-1 to the Prm1 region of the chromatin in HEL cells. The GATA family of transcription factors are so-called because they bind to a consensus A/TGATAA/G DNA sequence. GATA-1 interacts with the co-activator Friend of GATA (FOG)-1 to regulate several genes involved in megakaryocyte differentiation. GATA-1 is expressed in haematopoietic progenitor cells, erythrocytes, megakaryocytes, eosinophils and mast cells and is essential for normal erythropoiesis and megakaryopoiesis. Loss of megakaryocytic GATA-1 expression in mice resulted in aberrant proliferation and maturation of megakaryocyte cells. The Ets family of transcription factors consists of approximately 30 proteins that play a role in a variety of cellular processes such as differentiation, apoptosis and development. Family members Ets-1, Fli-1 and PU-1 play an important role in megakaryocytic and erythroid differentiation. Whilst Ets-1 is downregulated and exported from the nucleus during erythroid maturation, it promotes differentiation and maturation of megakaryocytes. ChIP assays have demonstrated that Ets-1 binds to proximal regions in the GPIIb, GPIX, and thrombopoietin receptor (MPL) promoters. Moreover, Ets-1 and GATA-1 activate promoters for rat platelet factor (PF)4 and human thrombopoietin receptor, or MPL. It is indeed notable that the promoters of these genes are characterized by closely spaced GATA-1 and Ets binding elements, similar to those identified herein in Prm1. Functional cooperativity among GATA-1, FOG-1 and specific Ets family members is required for efficient expression of the megakaryocytic-specific αIIb gene. Herein, we report that Prm1 of the human TP gene contains an upstream activator sequence that contains functional elements for GATA and Ets factors separated by 5 bp and that GATA-1 and Ets-1 functionally cooperate by binding to these elements, thereby increasing the expression of TPα in HEL cells. In addition, the ability of a 250 bp subfragment encoding the aforementioned GATA-1 and Ets-1 sites to act as an independent UAS in HEL cells was confirmed whereby it resulted in a 4-fold increase in reporter gene expression directed by the heterologous SV40 promoter. So, several critical regulatory regions have been identified within Prm1 of the TP gene, including two UAS and two URS and a proximal “core” Prm1 region. Specifically, we have identified four functional overlapping Sp1/Egr1 elements and a single NF-E2 element in the proximal Prm1 region, as well as functional GATA and Ets elements within the UAS, located between −7962 and −7859, that regulate basal Prm1 activity. It seems likely that cooperative binding of Sp1 to multiple sites in the proximal Prm1 is an important step required for initiation of transcriptional activity. Herein, it appears that over-expression of Egr1 inhibits the Sp1-mediated activation of Prm1, suggesting that it is the relative balance between Sp1 and Egr1 binding that determines its basal transcription. Additionally, the activity of Prm1 in the HEL megakaryocyte cell line is increased due to a functional NF-E2 element in the proximal promoter, as well as functional GATA and Ets elements in an UAS. It has been suggested that the haematopoietic-specific factors NF-E2 and GATA-1 stabilize the open nucleosomal structure of the β-globin gene following Sp1 binding. Additionally, interactions between Ets factors and Sp1 stabilize Sp1 binding to the alpha promoter. The functional characterization of Prm1 herein greatly increases knowledge of the factors regulating expression of the human TP gene. These data not only provide a molecular and genetic basis for understanding the role of TXA₂ and its receptor TP in haemostasis and vascular disease but also provide a rationale for understanding how altered numbers of TPs, such as through dysregulation of signaling by the trans-acting factors involved or indeed through genetic polymorphisms in Prm1 itself, contribute to such diseases. Furthermore, these data also provide predictive or prognostic diagnostic markers, which predict susceptibility to development of different diseases including different diseases of the cardiovascular system. Moreover, these data also significantly increase appreciation that expression of the individual TPα and TPβ isoforms, as products of Prm1 and Prm3, respectively, are subject to entirely distinct regulatory mechanisms. Amongst the transcription factor elements identified by bioinformatic analysis of URR1 were multiple GC-rich elements containing putative WT1 binding sites. WT1 has been reported to mediate repression of several gene promoters, including the Egr1 promoter, IGFI receptor, IGFII and PDGF-A, as well as mediating auto-repression of its own gene. Additionally, WT1 is thought to be an important factor in the regulation of haematopoiesis. Whilst it is highly expressed in a subset of CD34+ progenitors, it is down-regulated early in the course of differentiation of these cells. Additionally, WT1 mRNA is down-regulated during induction of erythroid and megakaryocytic differentiation of the K562 cell line. Recently, −KTS isoforms of WT1 have been confirmed to act as transcriptional regulators during haematopoiesis, where they activate transcription of the erythropoietin receptor. Moreover, increased expression of WT1 has been reported to occur in acute human leukemias, with evidence that WT1 expression is associated with prognosis. Considering the function of WT1 as a transcriptional repressor in many cases, as well as its role in haematopoietic differentiation, it was sought to determine whether WT1 can act as a repressor of Prm1 in HEL cells. Mutation of GC-rich elements containing putative overlapping WT1/Egr1/Sp1 binding sites, specifically at −8345, −8281, −8146 and −7831, alleviated repression of Prm1. Despite the indication that these GC elements mediate repression of Prm1, collective mutation of the sites resulted in de-activation of the promoter. As outlined in the model presented in FIG. 15, these mutational analyses suggest that repressor factor(s) normally bind to neighbouring GC elements at −8345, −8281, −8146 and −7831 in a cooperative manner, and it is suggested that mutation of any of these GC elements by SDM disrupts cooperative binding, thereby alleviating repression of Prm1. In the absence of repressor binding to the remaining intact sites, it is proposed that these elements may now have an increased affinity for factors, such as Egr1 or Sp1, that mediate activation, as opposed to WT1-mediated repression of Prm1. Therefore, it is suggested that disruption of remaining elements results in de-activation of the promoter, leading to the overall decrease in luciferase expression upon generation of Prm1^{GC}*^{(-7831,-8281,-8146,-7831)} from Prm1^{GC}*⁽⁻⁸³⁴⁵⁾. Evidence for this proposed model of cooperative binding comes from further studies whereby disruption of GC⁻⁷⁸³¹ in the Prm1B (−7962) sub-fragment, which does not contain any of the other four GC elements, actually decreased the luciferase activity directed by Prm1B. This effect is in contrast to the substantial increase in luciferase expression that occurred upon disruption of the same GC element within Prm1, where the other three GC elements at −8345, −8281 and −8146 were intact. The contrasting outcomes of disrupting the same element in two distinct Prm1 fragments with different 5′ sequences highlights the influence of cooperation among specific factors on binding to local promoter elements within Prm1. EMSAs using the Egr1 consensus sequence as a non-labelled competitor suggested that each of the four aforementioned GC elements has a sequence capacity to bind Egr1 and/or WT1, since both Egr1 and WT1 proteins have been widely reported to bind to the Egr1 consensus sequence. However, none of the complexes that bind to the four GC elements at −8345, −8281, −8146 and −7831 were competed by the WTE sequence, an element thought to be selectively bound by WT1. Collectively, these data suggested that in isolation from their surrounding sequences, the GC elements have a high binding affinity for Egr1 but not for WT1, or alternatively that the five GC elements have a binding affinity for WT1 isoforms that bind to Egr1 consensus elements but not to WTE sequences. Not surprisingly, GC⁻⁸³⁴⁸, GC⁻⁸²⁸¹ and GC⁻⁷⁸³¹ also have a sequence capacity to bind Sp1, since overlapping sites for Sp1 and Egr1/WT1 are frequently found in promoter sequences due to the similarity in their consensus elements. Interestingly, it has previously been reported that Sp1 binding to the GC element at −8345 mediates increased Prm1 activity in response to phorbol 12-myristate 13-acetate (PMA) in K562 cells. Therefore, it is suggested that this element may play a diverse role in Prm1 regulation. Due to the complex binding patterns of the Prm1-based probes and the GC elements in question, EMSA or supershift assays using Egr1, WT1 or Sp1 antibodies failed to provide a clear interpretation of the identities of specific transcription factors that bind to these sites in vitro. However, herein, those supershift assays were superseded by ChIP analysis, which revealed that endogenous WT1, but not Egr1 nor Sp1, are bound in vivo to the Prm1 region (from −8460 to −7607) of chromatin extracted from HEL cells. Moreover, ectopic over-expression of −KTS isoforms of WT1 led to modest, but significant, decreases in Prm1-directed luciferase expression and in TPα mRNA expression. It is suggested that only modest reductions in Prm1-directed luciferase expression were seen due to the already abundant endogenous expression of WT1 in HEL cells, and it is likely that over-expression of −KTS isoforms may have led to greater reductions in Prm1-directed gene expression if the total amount of transfected DNA herein was not limited by the luciferase-based reporter assay itself. Moreover, since the transcriptional effects of WT1 may be dependent on synergistic activity of more than one isoform of the protein, data from over-expression studies may not reflect the true extent of repression of Prm1 activity by WT1. Collectively, these data indicate that WT1 is the repressor factor that binds to the GC elements at −8345, −8281, −8146 and −7831. It is proposed that WT1 overcomes competition from other factors, such as Egr1 and/or Sp1, by a cooperative method of binding that relies on multiple neighbouring GC elements within Prm1 to exert its repression. Bioinformatic analysis of the two remaining URS regions within Prm1, from −6848 to −6648 and from −6258 to −6123, also revealed putative GC elements in each case. Mutational analysis of the GC elements in both regions, specifically at −6717 and −6206, indicated that they both mediate repression of Prm1. EMSAs to analyse WT1/Egr1/Sp1 binding to the −6717 and −6206 elements in vitro revealed that these elements had a sequence capacity to be bound by Sp1, Egr1 and/or WT1. More specifically, ChIP analysis revealed WT1 as the predominant protein bound to the −6848 to −6648 region in vivo, as well as indicating a major role for WT1 binding to RR3 (from −6258 to −6123) within the “core” Prm1. Data herein suggest that WT1 binds to the GC⁻⁶²⁰⁶ element within the proximal Prm1. Moreover, over-expression of the −KTS isoforms, specifically (+exon 5/−KTS) and (−exon 5/−KTS), repressed luciferase activity directed by Prm1D (−6848). In contrast to the cooperative and co-dependent manner in which WT1 binds to GC⁻⁸³⁴⁵,GC⁻⁸²⁸¹, GC⁻⁸¹⁴⁶ and GC⁻⁷⁸³¹, it seems that it binds to the −6717 and −6206 elements independently to mediate repression of Prm1. In this study, it was sought to identify the key cis-acting elements and trans-acting factors of the three distinct repressor regions (URR1, from −8500 to −7962; URR2, from −6848 to −6648; RR3, from −6258 to −6123). Herein, it is reported that the repression exerted within each of the three regions is largely attributable to the zinc finger transcription factor WT1. Considering the importance of TXA₂ and TP within the kidney, together with this novel role for WT1 as a repressor of Prm1, it is suggested that WT1 may play a role in regulation of Prm1 and TPα expression in the renal system and thus affords a predictive diagnostic marker for kidney disease where mutations in Prm1 WT1 binding site occurs. Moreover, WT1 triggers lineage-specific differentiation of human primary haematopoietic progenitor cells and WT1 mRNA is down-regulated during induction of erythroid and megakaryocytic differentiation of the K562 cell line. Therefore, it is suggested that down-regulation of WT1 may act to increase Prm1 activity, thereby increasing TPα expression, during megakaryocytic differentiation of HEL cells. NF-E2, GATA-1 and Ets-1 were previously identified as key regulators of Prm1 during megakaryocytic differentiation. Collectively, the data from these studies suggest that combinatorial gene regulation by WT1, GATA-1, Ets-1 and NF-E2 may be critical for regulation of TPα expression during different stages of megakaryocytic differentiation. A further aim of the current study was to investigate whether PMA-induced differentiation of the megakaryoblastic HEL 92.1.7 cell line is associated with increased Prm1 activity and TPα expression. Thereafter, it was sought to identify the specific factors that regulate Prm1-directed transcription and TPα expression during differentiation of HEL cells toward the megakaryocytic phenotype. Herein, PMA-induced differentiation of HEL cells led to substantial increases in TPα mRNA expression in a time-dependent manner, as well as leading to a 4-fold induction of Prm1-directed reporter gene expression. Moreover, reporter gene assays indicated that the increase in Prm1-directed luciferase expression in response to PMA is mediated mainly by cis-acting elements located between −8500 and −7504, whilst element(s) in the region from −7504 to −6848 play a more minor role. These elements consist of overlapping binding sites for WT1/Egr1/Sp1, and it has been confirmed that each has a sequence capacity to bind Egr1 and/or WT1 isoform(s), while GC⁻⁸³⁴⁵, GC⁻⁸²⁸¹ and GC⁻⁷⁸³¹ also have a sequence capacity to bind Sp1. Herein, it was sought to determine if one or more of these GC elements may be responsible for the PMA-mediated induction of Prm1 in HEL cells. Mutation of the individual elements each resulted in a marginal reduction of PMA-mediated induction of Prm1, while a substantial reduction was observed upon combined mutation of all four elements together to generate Prm1^{GC}*^{(-8345,-8281,-8146,-7831)}. These data indicate that GC elements at −8345, −8281, −8146 and −7831 act in an independent manner to mediate the PMA-induction of Prm1 transcriptional activity. Thereafter, it was sought to determine if the levels of expression of WT1, Egr1 or Sp1 changed upon PMA-induced differentiation of HEL cells. Western blot analysis indicated that the levels of expression of Sp1 or of the 52 kDa/54 kDa forms of WT1 were not substantially altered over a period of 48 h in response to PMA-mediated HEL cell differentiation. Conversely, there was a time-dependent, but transient, increase in Egr1 expression. PMA-mediated up-regulation of Egr1, as well as of other immediate early genes, is known to occur through activation of the ERK 1/2 signaling cascades, leading to phosphorylation of a subfamily of Ets-domain transcription factors known as the ternary complex factors (TCFs), which include Elk-1, SAP-1 and SAP-2. Herein, over-expression of the Egr1 co-repressor NGFI-A-binding protein 1 (NAB1) significantly reduced PMA-mediated induction of Prm1. NAB1 and NAB2 act as co-repressors of Egr1 activity by binding to an inhibitory domain within the Egr1 protein, thereby decreasing its transcriptional activity. Moreover, PD98059, a selective inhibitor of the ERK 1/2 pathway, significantly abrogated the PMA-mediated increase in Prm1 activity, as well as inhibiting the PMA-mediated induction of Egr1 protein expression in HEL cells. Therefore, it is indicated that PMA-induced differentiation of HEL cells is associated with an increase in Egr1 expression through ERK signaling, which in turn leads to increased Prm1-directed gene expression. Thereafter, it was sought to investigate whether the increased Prm1-directed gene expression could be due to increased binding of Egr1 to upstream GC-enriched elements at −8345, −8281, −8146 and −7831 within Prm1. Like Prm1, many gene promoters, including that of the human copper-zinc superoxide dismutase (SOD1) gene, contain GC elements for which WT1, Egr1 and Sp1 can compete with one another for binding. Recruitment and/or binding of a specific transcription factor depend mainly on its concentration within the nucleus, as well as its affinity for a specific cis-acting element. While WT1, Egr1 and Sp1 proteins are abundantly expressed in HEL cells, it was previously established that WT1 binds cooperatively to GC elements at −8345, −8281, −8146 and −7831 within Prm1 to repress its activity in quiescent HEL cells. Herein, ChIP analysis confirmed that WT1 was bound to the 5′ Prm1 region from −8460 to −8006 in non-stimulated HEL cells, with an absence of Egr1 or Sp1 binding. Following incubation of HEL cells with PMA for 5 h, there was some evidence of low-level binding of Egr1 to Prm1, while there was no appreciable change in the level of WT1 binding. However, following a longer incubation with PMA (8 h), the level of Egr1 bound to Prm1 had substantially increased, while WT1 binding was not detected. Hence, collectively, this study provided evidence that PMA-stimulation of HEL cells led to a time-dependent increase in Egr1 expression via the ERK pathway, leading to increased binding of Egr1 to upstream elements within Prm1, thereby activating the promoter. This increase in Egr1 binding to Prm1 was associated with a decrease in binding of the repressor WT1. Although it has been established that WT1 mRNA is down-regulated during induction of erythroid and megakaryocytic differentiation of the K562 cell line, no substantial decreases in WT1 expression were observed herein following PMA-stimulation of HEL cells. Therefore, it was investigated whether the intracellular localization of WT1 may be altered upon PMA-stimulation of HEL cells. It was established that in non-treated HEL cells and in HEL cells stimulated with PMA for 1 h, WT1 was almost exclusively localized to the nucleus while at 5 h and 8 h, there was a redistribution of WT1 to the cytosolic fraction. Thereafter, at 16 and 24 h, a higher proportion of WT1 was found in the cytosol than in the cytoplasm. Herein, the data indicate that translocation of WT1 from the nucleus to the cytoplasm, as well as increased competition from up-regulated Egr1, is responsible for decreased binding of WT1 to upstream GC elements within Prm1 in response to PMA-stimulated differentiation of HEL cells. Thereafter, ChIP analysis of HEL cells that were pre-incubated with PMA for a longer period (16 or 24 h) revealed a decrease in Egr1 binding to residual levels, whilst WT1 binding was still undetected. Conversely, substantial binding of Sp1 was detected. Western blot analysis revealed that, while the highest level of Egr1 expression was observed following incubation with PMA for 5 h −6 h, Egr1 expression levels decreased thereafter, possibly due to increased protein turnover, such that the level of expression in HEL cells following incubation with PMA for 24 h was not significantly different to that observed in vehicle-treated HEL cells. Moreover, since the transcriptional effects of Egr1 are largely determined by its interaction with specific co-activators, including CREB-binding protein (CBP) and p300, as well as co-repressors including NAB1 and NAB2, it is suggested that sustained PMA-stimulation of HEL cells may eventually interfere with specific interactions that promote Egr1 binding and activation of Prm1, thereby maintaining a negative feedback loop to regulate its activity. Furthermore, it has been established that PMA increases ERK-mediated phosphorylation of Sp1, enhancing its DNA-binding affinity. Moreover, it is thought that Sp1 may play a structural role in transcriptional activation by binding to multiple sites on promoter DNA to maintain chromatin in an accessible conformation. Hence, increased turnover of Egr1 protein, combined with increased competition from phosphorylated Sp1, is suggested to be responsible for the overall concomitant decrease in Egr1 binding and increase in Sp1 binding. Collectively, in the current study, chromatin immunoprecipitation (ChIP) analysis indicated a distinct pattern of binding of WT1, Egr1 and Sp1 proteins to Prm1 in HEL cells in response to PMA stimulation, which is thought to be responsible for the multi-phasic, sustained increases in Prm1-directed luciferase expression observed following PMA-stimulation of HEL cells over a 48 h period. Considering the data from mutational, western and ChIP analyses presented herein, as well as from analysis of intracellular localization of WT1, a model to explain the PMA-mediated induction of Prm1 can be proposed, as outlined in FIG. 25. It is suggested that following incubation of HEL cells with PMA for approximately 5 h, ERK-mediated up-regulation of Egr1 expression increases competition between WT1 and Egr1 for binding to 5′ GC elements. This leads to increased Egr1 binding and promoter activation. Subsequently, PMA-stimulation of HEL cells leads to translocation of WT1 from the nucleus to the cytoplasm, leading to promoter de-repression. The decrease in WT1 binding, as well as the continued increase in Egr1 expression, facilitates a further increase in Egr1 binding and a more pronounced activation of the promoter. Thereafter, it is suggested that increased turnover of Egr1, in association with increased competition for binding from phosphorylated Sp1, facilitates Sp1-mediated increases in Prm1 transcriptional activity in response to PMA over longer incubations. Moreover, Egr1 mediates increased expression of G_{αq} during PMA-induced megakaryocytic differentiation of HEL cells. G_{αq} plays a central role in platelet signal transduction, and platelets from G_{αq}-deficient mice are unresponsive to a variety of physiological platelet activators. Additionally, due to its induction by stimuli such as shear stress, mechanical injury, hypoxia and reactive oxygen species, Egr1 has been associated with the pathogenesis of several vascular diseases following injury to the vascular endothelium. Moreover, Egr1 and Egr1-induced genes are significantly up-regulated in endothelial and smooth muscle cells within human atherosclerotic lesions, while induction of atherosclerosis in low density lipoprotein (LDL)-null mice resulted in increased aortic expression of Egr1. Considering the well-documented role for Egr1 in megakaryocytic differentiation, it is likely that it may be responsible, at least in part, for the induction of Prm1 activity and TPα expression in response to PMA-mediated megakaryocytic differentiation of HEL cells. Moreover, following its up-regulation in vascular disease, increased transcriptional activity of Egr1 may be responsible for increased expression of TPα in various pathophysiologic conditions, including atherosclerosis. Phorbol ester stimulation of megakaryocytic differentiation of HEL cells provides a useful means of studying the molecular changes involved in the differentiation process. Herein, upon PMA-induced differentiation of HEL cells, there was a concomitant induction of Prm1 activity and a resultant increase in endogenous TPα mRNA expression in HEL cells. This increased TPα expression appears to be dependent on changes in the levels of Egr1, WT1 and Sp1 binding to Prm1. The finding that PMA-mediated differentiation of HEL cells results in increased Prm1-directed expression of TPα, together with the recognized importance of TPα in platelets, indicate that increased expression of TPα is a key step in megakaryocytic differentiation. Additionally, it is suggested that Vitamin D₃-induced differentiation of HEL cells may increase Prm1-directed TPα expression in a similar manner to PMA, since pre-incubation of HEL cells herein led to significant increases in Prm1-directed luciferase expression, as well as substantial increases in Egr1 protein expression. The current study also indicates an important role for Egr1 and Sp1 in regulating Prm1 activity during the differentiation process. Furthermore, it seems that the specific pattern of WT1, Egr1 and Sp1 binding to GC elements within Prm1 is critical for initial and sustained increases in TPα expression during the differentiation process. Collectively, the data provide a molecular basis for understanding the role of TXA₂ and TP in haemostasis and megakaryocytic differentiation toward the platelet phenotype, as well as providing further evidence to indicate that TPα is the more important of the two TP isoforms in haemostasis and platelet biology. In summary, the present invention describes a gene transcription-regulating polynucleotide comprising the nucleic acid sequence of SEQ ID NO:1 of the nucleic acid sequence of thromboxane A2 receptor promoter, or a fragment thereof, the gene transcription-regulating polynucleotide, or the fragment thereof, further comprising at least one nucleic acid modification and/or substitution, selective results of which are described in Table 6:

TABLE 6

| | Cell Lines | | | | |
|---|---|---|---|---|---|
| Mutation | HEL | EA.hy 926 | HEK293 | 1° hAoSMC | WI-38 |
| Prm1 → Prm1^{GC-8146}* | ↑ *** | ns | ns | ↑ ** | ns |
| Prm1 → Prm1B | ↑ *** | ↑ ** | ↑ * | ns | ns |
| Prm1D → Prm1D^{WT1(a), (b)}* | ↑ *** | ↑ *** | ↑ *** | ↑ *** | ↑ *** |

In brief, it can be seen that all three variants (Prm1^{GC-8146)}*, Prm1B and Prm1D^{WT1(a),(b)}*) of Prm1 yielded significantly stronger promoters in HEL cells. Prm1^{GC-8146}* and Prm1B fragments in comparison to Prm1 have variable results in the other cell lines (EA.hy 926, HEK, 1° hAoSMC & WI-38). In all cell lines, the double WT1 mutation (Prm1D^{WT1(a),(b)}*) results in a significantly stronger promoter than Prm1, Prm1B or Prm1D. The data is complemented by the immunoblotting analysis (page 8, of this document) wherein hTF protein expression driven by Prm1D^{WT1(a),(b)}* is significantly higher than that directed by CMV promoter. Furthermore, Prm1D^{WT1(a)}*^{(b)}*^{(X)}* is an even stronger promoter for driving luciferase expression relative to Prm1D^{WT1(a)}*^{(b)}* in HEL 92.1.7 cells.

In conclusion, the present invention describes a proprietary, universally strong promoter, particularly in the case of Prm1D^{WT1(a),(b)}*, even relative to the widely used SV40 promoter (in pGL3 control plasmid) or CMV promoter (in pcDNA3.1(−), whereas other Prm1-based fragments show cell specificity allowing low, medium or high expression depending on the cell type versus promoter fragment.

All nucleotide sequence position numbers, with the exception of the nucleotide sequence position numbers used in PatentIn (hereunder), are based human Prm1, when compared to the position with respect to ATG start site (A is at position +1) of the TP gene. The nucleotide sequence position numbers used in PatentIn start at nucleotide 1 instead of −8500.

### REFERENCES

HOFFMANN, E., ASHOURI, J., WOLTER, S., DOERRIE, A., DITTRICH-BREIHOLZ, O., SCHNEIDER, H., WAGNER, E. F., TROPPMAIR, J., MACKMAN, N. & KRACHT, M. (2008). Transcriptional regulation of EGR-1 by the interleukin-1-JNK-MKK7-c-Jun pathway. J Biol Chem, 283, 12120-8.
KOOREN, J., PALSTRA, R. J., KLOUS, P., SPLINTER, E., VON LINDERN, M., GROSVELD, F. & DE LAAT, W. (2007). Beta-globin active chromatin Hub formation in differentiating erythroid cells and in p45 NF-E2 knock-out mice. J Biol Chem, 282, 16544-52.
MURAKAMI, Y., YAMAGOE, S., NOGUCHI, K., TAKEBE, Y., TAKAHASHI, N., UEHARA, Y. & FUKAZAWA, H. (2006). Ets-1-dependent expression of vascular endothelial growth factor receptors is activated by latency-associated nuclear antigen of Kaposi's sarcoma-associated herpesvirus through interaction with Daxx. J Biol Chem, 281, 28113-21.
SAWADO, T., IGARASHI, K. & GROUDINE, M. (2001). Activation of beta-major globin gene transcription is associated with recruitment of NF-E2 to the beta-globin LCR and gene promoter. Proc Natl Acad Sci USA, 98, 10226-31.
SINHA-DATTA, U., HORIKAWA, I., MICHISHITA, E., DATTA, A., SIGLER-NICOT, J. C., BROWN, M., KAZANJI, M., BARRETT, J. C. & NICOT, C. (2004). Transcriptional activation of hTERT through the NF-kappaB pathway in HTLV-I-transformed cells. Blood, 104, 2523-31.
SOBUE, S., HAGIWARA, K., BANNO, Y., TAMIYA-KOIZUMI, K., SUZUKI, M., TAKAGI, A., KOJIMA, T., ASANO, H., NOZAWA, Y. & MURATE, T. (2005). Transcription factor specificity protein 1 (Sp1) is the main regulator of nerve growth factor-induced sphingosine kinase 1 gene expression of the rat pheochromocytoma cell line, PC12. J Neurochem, 95, 940-9.
SOHN, R. H., DEMING, C. B., JOHNS, D. C., CHAMPION, H. C., BIAN, C., GARDNER, K. & RADE, J. J. (2005). Regulation of endothelial thrombomodulin expression by inflammatory cytokines is mediated by activation of nuclear factor-kappa B. Blood, 105, 3910-7.
ZHANG, F., AHLBORN, T. E., LI, C., KRAEMER, F. B. & LIU, J. (2002). Identification of Egr1 as the oncostatin M-induced transcription activator that binds to sterol-independent regulatory element of human LDL receptor promoter. J Lipid Res, 43, 1477-85.
THIEL, G., SCHOCH, S. & PETERSOHN, D. (1994). Regulation of synapsin I gene expression by the zinc finger transcription factor zif268/egr-1. J Biol Chem, 269, 15294-301.
QUANDT, K., FRECH, K., KARAS, H., WINGENDER, E. & WERNER, T. (1995). MatInd and MatInspector: new fast and versatile tools for detection of consensus matches in nucleotide sequence data. Nucleic Acids Res, 23, 4878-84.
COYLE, A. T., MIGGIN, S. M. & KINSELLA, B. T. (2002). Characterization of the 5′ untranslated region of alpha and beta isoforms of the human thromboxane A2 receptor (TP). Differential promoter utilization by the TP isoforms. Eur J Biochem, 269, 4058-73.
   COYLE, A. T. & KINSELLA, B. T. (2005). Characterization of promoter 3 of the human thromboxane A receptor gene. A functional AP-1 and octamer motif are required for basal promoter activity. Febs J, 272, 1036-53.
TAJINDA, K., CARROLL, J. & ROBERTS, C. T., JR. (1999). Regulation of insulin-like growth factor I receptor promoter activity by wild-type and mutant versions of the WT1 tumor suppressor. Endocrinology, 140, 4713-24.
KOCH, C. M., ANDREWS, R. M., FLICEK, P., DILLON, S. C., KARAOZ, U., CLELLAND, G. K., WILCOX, S., BEARE, D. M., FOWLER, J. C., COUTTET, P., JAMES, K. D., LEFEBVRE, G. C., BRUCE, A. W., DOVEY, O. M., ELLIS, P. D., DHAMI, P., LANGFORD, C. F., WENG, Z., BIRNEY, E., CARTER, N. P., VETRIE, D. & DUNHAM, I. (2007). The landscape of histone modifications across 1% of the human genome in five human cell lines. Genome Res, 17, 691-707.
MIGGIN, S. M. & KINSELLA, B. T. (1998). Expression and tissue distribution of the mRNAs encoding the human thromboxane A2 receptor (TP) alpha and beta isoforms. Biochim Biophys Acta, 1425, 543-59.
SAMBROOK, J., FRITSCH, E. F. & MANIATIS, T. (1989). Molecular cloning: a laboratory manual. Cold Spring Harbor, N.Y.: Cold Spring Harbor Laboratory.

1-55. (canceled)

56. A gene transcription-regulating polynucleotide comprising
(a) the nucleic acid sequence of SEQ ID NOs:2-6, optionally comprising at least one nucleic acid modification or substitution;
(b) the nucleic acid sequence of a fragment of SEQ ID NO:1 of the nucleic acid sequence of thromboxane A2 receptor promoter, optionally comprising at least one nucleic acid modification or substitution; or
(c) the nucleic acid sequence of SEQ ID NO:1 of the nucleic acid sequence of thromboxane A2 receptor promoter comprising at least one nucleic acid modification or substitution.

57. The gene transcription-regulating polynucleotide of claim 56, wherein the at least one nucleic acid modification or substitution is present, and is located within:
(a) SEQ ID NOs:2-6;
(b) SEQ ID NO:1 or the fragment thereof, but not located within SEQ ID NOs:2-6; or
(c) SEQ ID NO:1 or the fragment thereof, and at least one of SEQ ID NOs:2-6.

58. The gene transcription-regulating polynucleotide of claim 56, wherein at least one nucleic acid modification or substitution is present, and is introduced at least one location within SEQ ID NO:1 of the nucleic acid sequence of thromboxane A2 receptor promoter, wherein the location of the 5′ most nucleotides of the modification or substitution are at or adjacent to nucleic acid positions selected from the group of: −6007, −6022, −6080, −6098, −6206, −6278, −6294, −6717, −7805, −7870, −7890, −7831, −8146, −8281 and −8345 of the promoter.

59. The gene transcription-regulating polynucleotide of claim 56, wherein the at least one nucleic acid modification or substitution is present and is introduced at least one location within SEQ ID NO:1 of the nucleic acid sequence of thromboxane A2 receptor promoter, wherein the location of the 5′ most nucleotides of the modification or substitution are at or adjacent to nucleic acid positions selected from the group of: −8500, −7962, −7717, −6848, and −6320 of the promoter.

60. The gene transcription-regulating polynucleotide of claim 56, wherein at least one nucleic acid modification or substitution is present, and is introduced into at least one element, wherein the element is selected from the group of SEQ ID NO:2, SEQ ID NO:3, SEQ ID NO:4, SEQ ID NO:5, and SEQ ID NO:6.

61. The gene transcription-regulating polynucleotide of claim 60, wherein the nucleic acid modification is selected from the group of:
(a) a multiplication of at least one nucleic acid or element,
(b) an insertion of at least one nucleic acid or element,
(c) a deletion of at least one nucleic acid or element,
(d) an inversion of the element, and
(e) a nucleic acid substitution or modification within the element.

62. The gene transcription-regulating polynucleotide of claim 60, wherein the element is:
(a) selected from the group of elements listed in Table 2;
(b) a binding site for a transcription factor selected from the group consisting of GC, GATA, Ets, Sp1, Egr1, NF-E2, WT-1, and AP1;
(c) a nucleic acid sequence selected from the group of SEQ ID NOs: 8, 10, 12, 14, 16, 18, 20, 22, 24, 26, 28, 30, 32, 34, and 36, as indicated under the Wild-type Sequence (5′ to 3′) column in the Table 1; or
(d) a nucleic acid sequence selected from the group of SEQ ID NOs: 9, 11, 13, 15, 17, 19, 21, 23, 25, 27, 29, 31, 33, 35, and 37, as indicated under the Mutated Sequence column in the Table 1.

63. The gene transcription-regulating polynucleotide of claim 56, wherein the gene transcription-regulating polynucleotide further comprises at least one element comprising a nucleic acid sequence, which facilitates initiation of transcription; and optionally wherein the element comprises a nucleic acid sequence selected from the nucleic acid sequence defined in SEQ ID NO 7 or a fragment thereof; and the nucleic acid sequence of the human cytomegalovirus (CMV) immediate-early enhancer and promoter.

64. The gene transcription-regulating polynucleotide of claim 63 comprising:
(a) the nucleic acid sequence of SEQ ID NO 7 or a fragment thereof, and
(b) at least one nucleic acid sequence selected from the group consisting of SEQ ID NOs 2-6.

65. The gene transcription-regulating polynucleotide of claim 63 comprising:
(a) the nucleic acid sequence of SEQ ID NO 7 or a fragment thereof, and
(b) at least one nucleic acid modification or substitution, or at least one further nucleic acid introduced at least one location within SEQ ID NO 1 of the nucleic acid sequence of thromboxane A2 receptor promoter, the at least one location comprising locations whose 5′ most nucleotides are at or adjacent to nucleic acid positions −6007, −6022, −6080, −6098, −6206, −6278, −6294, −6717, −7805, −7870, −7890, −7831, −8146, −8281 or −8345 of the promoter.

66. The gene transcription-regulating polynucleotide of claim 56, comprising at least one of SEQ ID NO:2 or SEQ ID NO 3.

67. The gene transcription-regulating polynucleotide of claim 56, comprising at least one of SEQ ID NO 4, SEQ ID NO 5, or SEQ ID NO 6.

68. The gene transcription-regulating polynucleotide of claim 66, further comprising at least one further nucleic acid modification or substitution at one or more locations whose 5′ most nucleotides are at or adjacent to positions: −7805, −7870, −7890, or −7831 of the promoter.

69. The gene transcription-regulating polynucleotide of claim 67, further comprising at least one further nucleic acid modification or substitution at one or more locations whose 5′ most nucleotides are at or adjacent to positions: −8345, −8281, −8146, or −6717 of the promoterjm.

70. The gene transcription-regulating polynucleotide of claim 56, wherein said polynucleotide comprises:
(a) the nucleic acid sequence of SEQ ID NO:1 with a nucleic acid modification or substitution at position −8146;
(b) the nucleic acid sequence of SEQ ID NO:13;
(c) the nucleic acid sequence defined by nucleotide positions −7962 to −5895 of SEQ ID NO:1; or
(d) the nucleic acid sequence defined by nucleotide positions −6848 to −5895 of SEQ ID NO:1.

71. The gene transcription-regulating polynucleotide of claim 70, further comprising at least one nucleic acid modification or substitution selected from the group of:
(a) a nucleic acid modification or substitution at a position selected from nucleic acid positions −6717, −6206, and −6800;
(b) a G→C nucleic acid substitution at position −6800,
(c) a CC→AT nucleic acid substitution at position −6717, and
(d) a CC→TA nucleic acid substitution at position −6206.

72. A method for regulating transcription of a gene, the method comprising providing a gene transcription-regulating polynucleotide of claim 56 in operable association with the gene, optionally within a host cell.

73. A method of diagnosing a disorder caused by, or associated with, dysregulated thromboxane A2 signalling, the method comprising the steps of:
(a) identifying a nucleic acid modification or substitution within SEQ ID NO:1 of the nucleic acid sequence of the promoter of thromboxane A2 receptor, and
(b) associating the presence of the nucleic acid modification or substitution with a disorder caused by, or associated with, dysregulated thromboxane A2 signalling.

74. The method of claim 73, wherein the disorder comprises a vascular disorder, a neoplastic disorder, preterm labor, pre-eclampsia, or a renal disorder.

75. A method for treating a patient suffering from a disorder caused by, or associated with, dysregulated thromboxane A2 signalling, the method comprising the step of:
(a) rendering Prm1 non-functional, or
(b) rendering genetically mutated Prm1 functionally normal with respect to
(i) the pattern of Prm1 transcription in human cells and tissues or
(ii) the quantification of expression to reflect that found in normal cells or tissues.
